(19) United States
(12) Patent Application Publication
(10) Pub. No.: US 2022/0226335 A1
(43) Pub. Date: Jul. 21, 2022

(54) Non-Peptidic Heterocycle-Containing Compounds for the Treatment of Alzheimer?s Disease

(71) Applicant: The Governors of the University of Alberta, Edmonton, Alberta (CA)

(72) Inventors: Jack Jhamandas, Edmonton (CA); Wen Fu, Edmonton (CA); Ryoichi Kimura, Sanyo-Onoda (JP); D. Lorne Tyrrell, Edmonton (CA); Ana Clementin, Edmonton (CA); Kamlesh Sahu, Edmonton (CA); Alexandr Belovodskiy, Edmonton (CA); Mostofa Hena, Edmonton (CA); Bing Bai, Edmonton (CA); Appan Srinivas Kandadai, Edmonton (CA); James A. Nieman, Sherwood Park (CA); Michael Houghton, Edmonton (CA)

(21) Appl. No.: 17/605,194

(22) PCT Filed: Apr. 23, 2020
(86) PCT No.: PCT/CA2020/050537

§ 371 Date: Oct. 20, 2021

Related U.S. Application Data
(60) Provisional application No. 62/837,845, filed on Apr. 24, 2019.

(51) Int. Cl. A61K 31/538; A61K 31/513; A61K 31/5377; A61K 31/5513; A61P 25/28

(52) U.S. Cl. CPC A61K 31/538; A61K 31/513; A61P 25/28; A61K 31/5513; A61K 31/5377

(57) ABSTRACT

The present disclosure provides non-peptidic heterocycle-containing amylin receptor antagonist compounds, compositions that include the subject compounds, methods for preparing and using the amylin receptor antagonists, and compositions containing the amylin receptor antagonists for treating, preventing, or ameliorating Alzheimer's disease. Aspects of the present disclosure include a method of inhibiting activity of an amylin receptor by administering to a subject in need thereof a therapeutically effective amount of an amylin receptor antagonist.

### CROSS-REFERENCE TO RELATED APPLICATIONS

This application claims the benefit of U.S. Provisional Application No. 62/837,845, filed Apr. 24, 2019, the disclosure of which is incorporated herein by reference.

### INTRODUCTION

Alzheimer's disease is the most common form of dementia that is characterized by deposition of amyloid β-protein (A) intra- and extracellularly within cortical and limbic brain structures critical for memory and cognitive functions (Selkoe, 1994 and 2013; Hardy et al., 2002). A central question in Alzheimer's disease research is whether the amyloid protein is a cause or a consequence of the disease. Presently, it appears that the likely answer is both (Hardy, 2009). Evidence strongly supports a role for Aβ in the pathogenesis of Alzheimer's disease, namely: a) Alzheimer's disease associated with inherited Amyloid Precursor Protein (APP) mutations; b) neurotoxicity of soluble oligomeric Aβ when applied to neurons; and c) APP overexpressing mice that recapitulate certain neuropathological and behavioral features of Alzheimer's disease (Liu et al., 2012; Bateman et al., 2012; Patel et al., 2012; Danysz et al., 2012). On the other hand, adverse events in clinical trials for Alzheimer's disease using Aβ vaccine-based therapy, and the subsequent failure of monoclonal antibody therapies and inhibitors of the Aβ generating gamma-secretase enzyme in improving cognitive functions in patients have forced reconsideration of these approaches as disease-modifying treatment strategies in Alzheimer's disease (Liu et al., 2012). Nonetheless, it is hard to imagine a definitive treatment that will not serve to ameliorate in some form the neurotoxic effects of Aβ, since this is a key “upstream” event in Alzheimer's disease pathogenesis (as established by alterations in CSF Aβ levels decades before clinical onset) (Bateman et al., 2012).

Multiple receptors have been implicated in mediating Aβ disruption of neuronal and synaptic processes in Alzheimer's disease, and thus identified as potential targets for developing anti-Aβ therapies (Patel et al., 2012; Danysz et al., 2012). The amylin receptor, comprised of heterodimers of the calcitonin receptor with receptor activity-modifying proteins, serves as a portal for the expression of deleterious effects of Aβ and human amylin (Fu et al., 2012). Amylin is a 37-amino acid peptide hormone that is co-secreted with insulin by beta cells of the pancreas that control glucose levels in blood.

Both Aβ and human amylin are amyloidogenic peptides which share structure-functional relationships; for example, both peptides aggregate and form soluble and insoluble oligomeric intermediates. Amylin has the propensity to aggregate and form amyloid oligomers and fibrils in the pancreas in type 2 diabetes (Westermark et al., 2011) and in Alzheimer's disease brains (Abedini et al., 2013). Aβ and human amylin cause dysfunction and death of neurons preferentially affected in Alzheimer's disease (Jhamandas et al., 2011; 2004). Neurotoxic effects of human amylin and Aβ are expressed through the amylin receptor 3 subtype (AMY3).

Amylin receptor antagonists, such as AC253 (a 24-amino acid peptide), are neuroprotective against Aβ toxicity (Jhamandas et al., 2004; 2011; 2012). Down-regulation of amylin receptor gene expression using siRNA mitigates oligomerized Aβ-induced toxicity (Jhamandas et al., 2011). In Alzheimer's disease transgenic model mice (TgCRND8) which over-express Aβ, amylin receptor was up-regulated within specific brain regions that demonstrate an increased burden of amyloid beta deposits (Jhamandas et al., 2011). Blockade of the amylin receptor with AC253 can reverse impairment of Aβ- or human amylin-induced depression of long-term potentiation, a cellular surrogate of memory, as observed in the hippocampus of Alzheimer's disease mice (TgCRND8) (Kimura et al., 2012). Similar benefits have been reported with pramlintide, a synthetic non-amyloidogenic analog of amylin. While data support a neuroprotective role for this compound, it appears to act as an amylin receptor antagonist rather than an agonist (Kimura et al., 2016). Although amylin receptor antagonist AC253 peptide has therapeutic potential in Alzheimer's disease, it suffers from poor enzymatic stability and an inability to penetrate the blood brain barrier.

### SUMMARY

The present disclosure provides non-peptidic heterocycle-containing amylin receptor antagonists, compositions that include the subject compounds, and methods for preparing and using the amylin receptor antagonists and the compositions for treating, preventing, or ameliorating Alzheimer's disease.

Aspects of the present disclosure include a method of inhibiting activity of an amylin receptor. The method includes administering to a subject in need thereof, a therapeutically effective amount of a compound of formula (I):

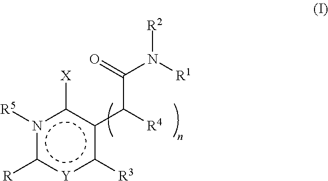

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² comprise a heterocycle, substituted heterocycle, fused-heterocycle or substituted fused-heterocycle;

R³ is selected from the group consisting of C₁-C₆-alkyl, substituted C₁-C₆-alkyl, C₃-C₆-cycloalkyl, substituted C₃-C₆-cycloalkyl, —CF₃, phenyl, and substituted phenyl;

each R⁴ is independently selected from the group consisting of —H and —CH₃;

R⁵ is present or absent, and if present is selected from the group consisting of —H and —CH₃;

each R⁶ is independently selected from the group consisting of —H, —CH₃, and —CH₂CH₃;

n is an integer from 1 to 3;

X is selected from the group consisting of ═O, ═NH, and —OCH₃;

Y is selected from the group consisting of —N═ and —CH═; and

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, the administering is effective for reducing cyclic AMP signal production in a cell.

In certain embodiments, the amylin receptor is an AMY3 receptor.

In certain embodiments, the administering is effective for producing a neuroprotective effect against amylin and/or amyloid-beta protein induced neurotoxicity.

In certain embodiments, the administering is effective for treating a disease mediated through activity of the amylin receptor. In certain embodiments, the disease is Alzheimer's disease.

In certain embodiments, the compound is of formula (II):

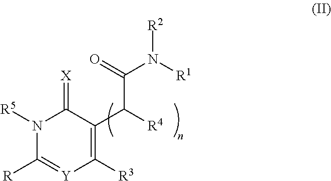

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle;

R³ is selected from the group consisting of C₁-C₆-alkyl, substituted C₁-C₆-alkyl, C₃-C₆-cycloalkyl, substituted C₃-C₆-cycloalkyl, —CF₃, phenyl, and substituted phenyl;

each R⁴ is independently selected from the group consisting of —H and —CH₃;

R⁵ is selected from the group consisting of —H and —CH₃;

each R⁶ is independently selected from the group consisting of —H, —CH₃, and —CH₂CH₃;

n is an integer from 1 to 3;

X is selected from the group consisting of ═O, and ═NH;

Y is selected from the group consisting of —N═ and —CH═; and

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, the compound is of formula (III):

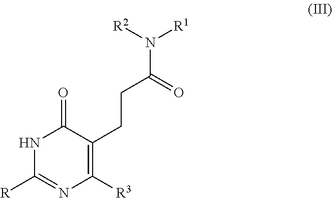

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle;

R³ is selected from the group consisting of C₁-C₆-alkyl, substituted C₁-C₆-alkyl, C₃-C₆-cycloalkyl, substituted C₃-C₆-cycloalkyl, and —CF₃; and each R⁶ is independently selected from the group consisting of —H, —CH₃, and —CH₂CH₃;

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, R is heterocyclyl or substituted heterocyclyl.

In certain embodiments, R¹ is —H or —CH₃.

In certain embodiments, R² is selected from the group consisting of C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle.

In certain embodiments, R¹ and R² together comprise a heterocycle, substituted heterocycle, fused-heterocycle or substituted fused-heterocycle.

In certain embodiments, R³ is —CH₃ or —CF₃.

In certain embodiments, the compound is selected from:

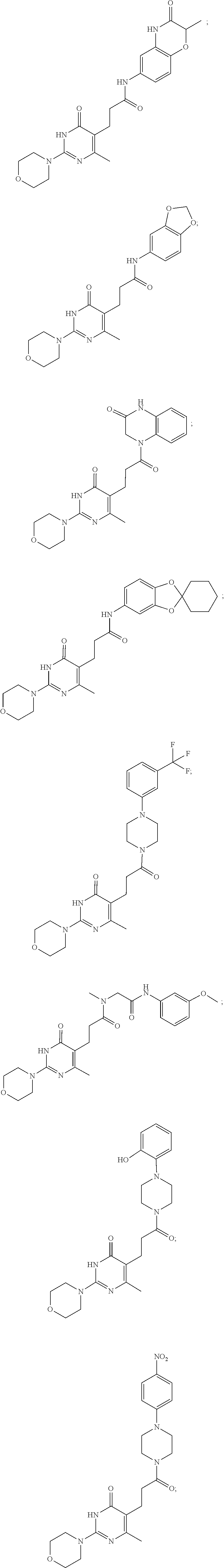

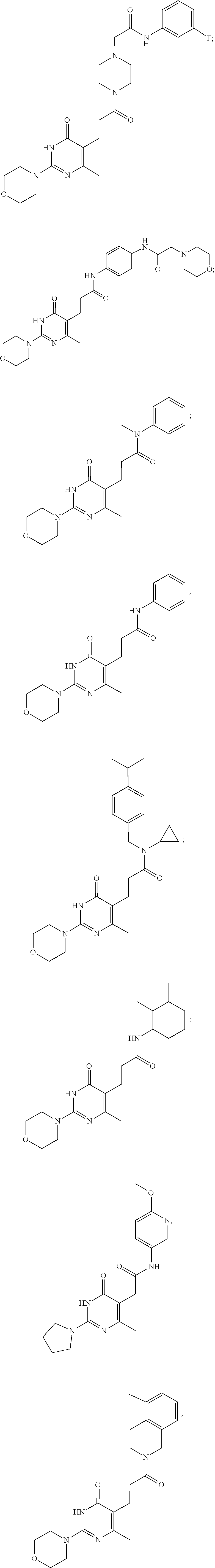

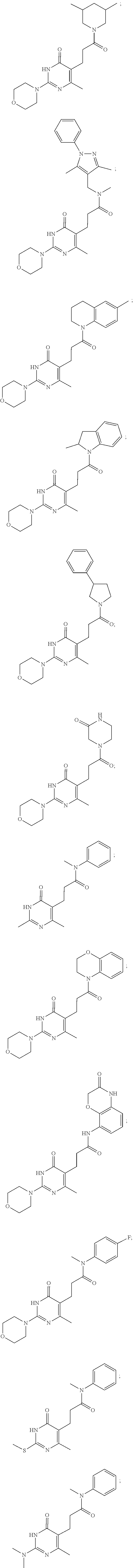

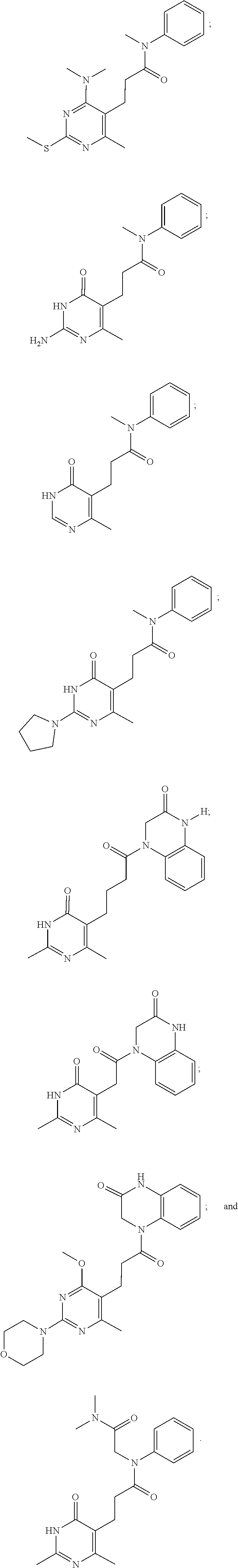

Aspects of the present disclosure include a compound of formula (IV):

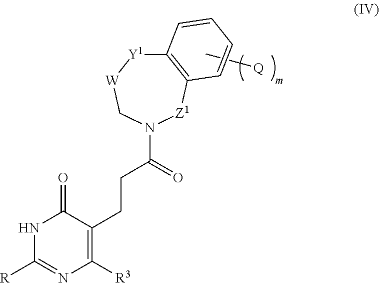

wherein:

R is selected from the group consisting of —H, C₁-C₃-alkyl, substituted C₁-C₃-alkyl, C₃-C₆-cycloalkyl, substituted C₃-C₆-cycloalkyl, heterocyclyl, substituted heterocyclyl, aryl, substituted aryl, —NHC(═O)R⁹, —N(R⁹)₂, —OR⁹, and —SR⁹;

R³ is selected from the group consisting of C₁-C₆-alkyl, C₃-C₆-cycloalkyl, and —CF₃;

m is 0, 1 or 2;

W is selected from the group consisting of —C(═O)— and —CH₂—;

each Q is independently selected from the group consisting of —F, —Cl, —CN, —CF₃ and C₁-C₃-alkyl;

Y¹ is selected from the group consisting of —NH—, —N(CH₃)—, —N(CH₂CH₃)— and —N(cyclopropyl)-;

each R⁹ is independently selected from the group consisting of —H, —CH₃, —CH₂CH₃ and cyclopropyl; and

Z¹ is absent or is —CH₂—;

or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof;

with the proviso that the compound is not:

4-[3-[1,6-dihydro-4-methyl-2-(4-morpholinyl)-6-oxo-5-pyrimidinyl]-1-oxopropyl]-3,4-dihydro-2(1H)-quinoxalinone, 6-methyl-2-(4-morpholinyl)-5-[3-oxo-3-(1,2,3,5-tetrahydro-1-methyl-4H-1,4-benzodiazepin-4-yl)propyl]-4(3H)-pyrimidinone, or 5-[3-(3,4-dihydro-4-methyl-1(2H)-quinoxalinyl)-3-oxopropyl]-6-methyl-2-(4-morpholinyl)-4(3H)-pyrimidinone.

In certain embodiments, Z¹ is absent.

In certain embodiments, m is 1. In certain embodiments, m is 0.

In certain embodiments, Q is —F, —CF₃, or —CH₃.

In certain embodiments, W is —C(═O)—.

In certain embodiments, Y¹ is —NH—. In certain embodiments, Y¹ is —NCH₃—.

In certain embodiments, R is selected from the group consisting of —H, C₁-C₃-alkyl, C₃-C₆-cycloalkyl, heterocyclyl, aryl, —NHC(═O)R⁹, —N(R⁹)₂, —OR⁹, and —SR⁹.

In certain embodiments, R is phenyl. In certain embodiments, R is a heterocyclyl. In certain embodiments, R is azetidinyl, pyrrolidinyl or piperidinyl. In certain embodiments, R is morpholinyl. In certain embodiments, R is —N(CH₃)₂ or —N(CH₂CH₃)₂. In certain embodiments, R is —OCH₃, —OCH₂CH₃, —SCH₃ or —SCH₂CH₃. In certain embodiments, R is C₁-C₃-alkyl or C₃-C₆-cycloalkyl. In certain embodiments, R is —CH₂CH₃. In certain embodiments, R is —CH₃. In certain embodiments, R is cyclopropyl.

In certain embodiments, R³ is —CF₃. In certain embodiments, R³ is —CH₃.

In certain embodiments, the compound is selected from:

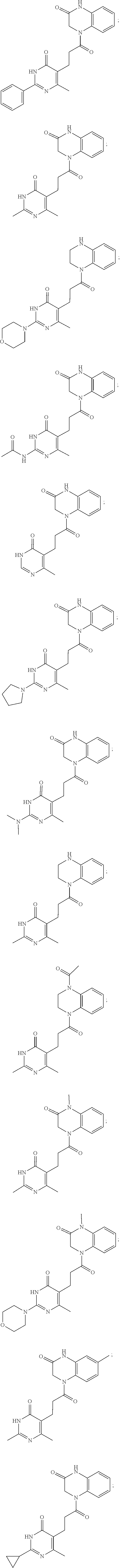

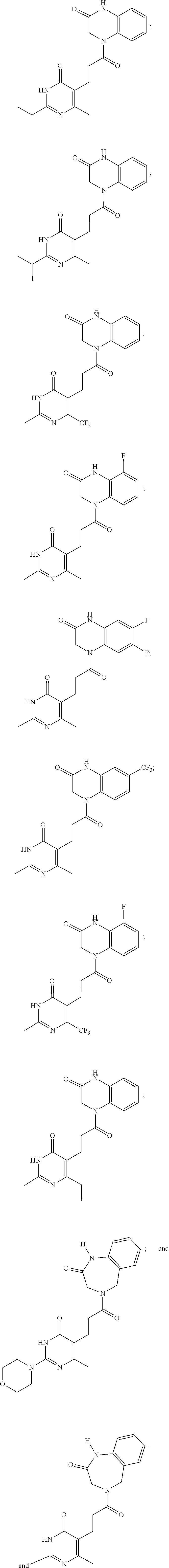

Aspects of the present disclosure include a method of inhibiting activity of an amylin receptor, where the method includes administering to a subject in need thereof, a therapeutically effective amount of a compound of formula (IV) of the present disclosure.

In certain embodiments, the administering is effective for reducing cyclic AMP signal production in a cell.

In certain embodiments, the amylin receptor is an AMY3 receptor.

In certain embodiments, the administering is effective for producing a neuroprotective effect against amylin and/or amyloid-beta protein induced neurotoxicity.

In certain embodiments, the administering is effective for treating a disease mediated through activity of the amylin receptor. In certain embodiments, the disease is Alzheimer's disease.

### BRIEF DESCRIPTION OF THE DRAWINGS

FIG. 1. Screening of the initial set of 10 compounds using a cAMP assay in amylin receptor subtype 3 expressing cells yielded Compound 3 as an amylin receptor antagonist.

FIGS. 2A and 2B. In mouse and human neuronal cell lines co-application of Compound 3 blunted human amylin and amyloid beta induced cytotoxicity.

FIGS. 3A-3D. In brain hippocampal slices, Compound 3 application at 1 μM blocks human amylin-induced depression of LTP (FIGS. 3A-3B). In hippocampal brain slices from transgenic AD mice (TgCRND8), LTP is chronically depressed. Application of Compound 3 increases LTP levels (FIGS. 3C-3D) to those seen in age matched control mice.

FIG. 4. Effect of Compounds 5-11 (Compound 3 analogues) on cAMP levels.

FIGS. 5A and 5B. Compound 3IH (in house synthesized Compound 3) produced effects identical to those seen with Compound 3 in blocking human amylin (hAM) generated cAMP responses (FIG. 5A). In cytotoxicity assays using human neuronal cell line (SK-N-SH) and primary cultures of human fetal neurons (HFNs), both Compound 3IH and Compound 3 demonstrate identical neuroprotective effects (FIG. 5B).

FIG. 6. Compound 23 was identified as most potent of these four analogues based on cAMP assay and downstream phosphoERK response.

FIGS. 7A-7B. Compound 23 is neuroprotective against amyloid beta toxicity in mouse (FIG. 7A) and human neuronal cell lines (FIG. 7B).

FIG. 8. Compound 23 and cyclized AC253 but not Compound 14 decreases total Aβ plaques and the area covered by plaques.

FIG. 9 shows dose-response relationship of Compound 23 against human amylin (at two concentrations) generated cAMP responses.

FIG. 10 shows a graphs of data from compounds of the present disclosure tested in a cyclic AMP (cyclic adenosine monophosphate, cAMP) assay in amylin receptor subtype 3 expressing cells.

FIG. 11 shows a schematic of a hippocampal long term potentiation (LTP) electrophysiology assay, according to embodiments of the present disclosure.

FIGS. 12A-12B. In a hippocampal LTP electrophysiology assay, Compound 23 at 1 μM restored the reduction in LTP by nanomolar dose of human amylin (h-Amylin) to control levels (FIG. 12A). FIG. 12B shows a graph of composite data showing Compound 23 blocked human amylin effects on LTP (n=6 in each group).

FIGS. 13A-13B. The reduction in LTP caused by nanomolar dose of amyloid beta (Aβ) was restored to control levels by 1 μM Compound 23 (n=5 in each group) (FIG. 13A).

FIG. 13B shows a graph of composite data showing Compound 23 blocked amyloid beta (Aβ) effects on LTP (n=6 in each group).

FIGS. 14A-14B. In aged (8 months+) transgenic AD mice (TgCRND8) low levels of basal LTP were restored to levels comparable to those seen in age-matched wild type (WT) littermate control mice (n=7 for each group) (FIG. 14A). FIG. 14B shows a graph of composite data showing Compound 23 restoration of LTP in AD mice to levels comparable to wild type mice (n=6 in each group).

FIGS. 15A-15B. An inactive compound (AVI9030; methyl N-[(1S)-2-methyl-1-[[(2S)-2-(5-phenyl-1H-imidazol-2-yl)-1-pyrrolidinyl]carbonyl]propyl]carbamate) did not block human amylin-induced reduction of LTP (FIG. 15A). FIG. 15B shows a graph of composite data showing an inactive compound was unable to block of human amylin effects on LTP (n=6 in each group).

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like, where R¹⁰ is chosen from chosen from hydrogen, optionally substituted alkyl, cycloalkyl, alkenyl, cycloalkenyl, alkynyl, aryl, heteroaryl and heterocyclic. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “alkoxycarbonylamino” refers to the group —NR^{d}C(O)OR^{d} where each R^{d} is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR²¹SO₂R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NR^{m}R^{m} where each R^{m} is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, heterocyclyl, aryl, or heteroaryl, provided that the point of attachment is through the non-aromatic ring. Fused ring systems include compounds where two rings share two adjacent atoms. In fused heterocycle systems one or both of the two fused rings can be heterocyclyl. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, 1,2,3,4-tetrahydroquinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, 3,4-dihydro-1,4-benzoxazine, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂ O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R^{7 0}, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R^{7 0}, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

By “treating” or “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the subject no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state or prophylactic treatment of a subject; (ii) inhibition, that is, arresting the development or further development of clinical symptoms, e.g., mitigating or completely inhibiting an active disease; and/or (iii) relief, that is, causing the regression of clinical symptoms or alleviating one or more symptoms of the disease or medical condition in the subject.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

As used herein, the term “amylin” refers to a 37 amino acid peptide hormone which is co-secreted with insulin from the pancreatic R-cell.

As used herein, the term “amyloid-beta protein” refers to peptides of 36-43 amino acids resulting from cleavage of the amyloid precursor protein, and which form the main component of neurotoxic amyloid plaques found in the brains of Alzheimer patients.

As used herein, the term “amylin receptor” refers to a receptor complex which binds amylin and amyloid-beta protein. The amylin receptor includes the calcitonin receptor (CTR) dimerized with one of three known subtypes of receptor activity-modifying protein (RAMP1, RAMP2, RAMP3). Both amylin (HA) and amyloid-beta protein (Aβ42) bind and directly activate the amylin receptor and trigger biological and neurotoxic effects. (Jhamandas et al., 2004).

As used herein, the term “amylin receptor antagonist” refers to a compound useful as an antagonist of the amylin receptor, or which binds to, but does not activate, the amylin receptor. The amylin receptor antagonist displaces and blocks the binding of amylin or amyloid-beta protein to the amylin receptor, thereby inhibiting the activity of amylin or amyloid-beta protein.

As used herein, the term “AC253” refers to a peptide antagonist of the amylin receptor. The “AC” prefix indicates the peptide's identity within the peptide library of Amylin Pharmaceuticals Inc. As used herein, the term “AC253” refers to a peptide having the amino acid sequence of SEQ ID NO: 1 (Ac-LGRLSQELHRLQTYPRTNTGSNTY) and which is capable of binding to the amylin receptor, thereby inhibiting the activity of amylin, amyloid-beta protein, or both.

As used herein, the term “chronic administration” refers to repeated administration of a compound to a subject. In such treatment, the compound can be administered at least once a week, such as at least once a day, or at least twice or three times a day for a period of at least one month, such as for example five months or more.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides non-peptidic heterocycle-containing amylin receptor antagonists, compositions that include the subject compounds, and methods for preparing and using the amylin receptor antagonists and the compositions for treating, preventing, or ameliorating Alzheimer's disease.

### Compounds and Methods of Treatment

The present disclosure provides methods of inhibiting activity of an amylin receptor. Embodiments of the present disclosure thus relate to methods and uses of the compounds disclosed herein as amylin receptor antagonists which bind to, but do not activate, the amylin receptor. Compounds of the present disclosure may be used to displace and/or block the binding of amylin or amyloid-beta protein to the amylin receptor, thereby inhibiting the activity of amylin or amyloid-beta protein. In some instances, compounds of the present disclosure are capable of binding to the AMY1 receptor. In some instances, compounds of the present disclosure are capable of binding to the AMY2 receptor. In some instances, compounds of the present disclosure are capable of binding to the AMY3 receptor. In some instances, compounds of the present disclosure are capable of binding to the AMY1 and AMY2 receptors. In some instances, compounds of the present disclosure are capable of binding to the AMY1 and AMY3 receptors. In some instances, compounds of the present disclosure are capable of binding to the AMY2 and AMY3 receptors. In some instances, compounds of the present disclosure are capable of binding to the AMY1, AMY2 and AMY3 receptors. As used herein, “AMY1 receptor” refers to a heterodimeric complex of the calcitonin receptor and RAMP1. As used herein, “AMY2 receptor” refers to a heterodimeric complex of the calcitonin receptor and RAMP2. As used herein, “AMY3 receptor” refers to a heterodimeric complex of the calcitonin receptor and RAMP3.

The amylin receptor antagonist may be used to reduce incidence of, reduce, treat, diminish, or prevent a disease or disorder in a subject where it is of benefit to reduce amylin or amyloid-beta protein activity. In certain embodiments, the disease is Alzheimer's disease. Therapeutic uses of compounds of the present disclosure in diseases or disorders, methods of prevention or treatment using compounds of the present disclosure, and uses of compounds of the present disclosure to prepare medicaments for therapeutic use are also included in embodiments of the present disclosure. In certain instances, embodiments of the present disclosure relate to the therapeutic use of compounds of the present disclosure in humans.

In certain embodiments, a method of treating, preventing, or ameliorating a disease or disorder in a subject is provided, where the method includes administering to the subject a therapeutically effective amount of one or more compounds of the present disclosure or a composition including same. As used herein, the term “disease” includes, but is not limited to, Alzheimer's disease. An effective amount of the compound or composition may be an amount sufficient to provide either subjective relief of symptoms or an objectively identifiable improvement as noted by a clinician or other qualified observer. As such, methods of “treating”, “preventing” or “ameliorating” refer to interventions performed with the intention of alleviating the symptoms associated with, preventing the development of, or altering the pathology of a disease, disorder or condition, such as Alzheimer's disease. Thus, in various embodiments, the methods of the present disclosure may include the prevention (prophylaxis), moderation, reduction, or curing of a disease, disorder or condition at various stages, such as for example Alzheimer's disease. In various embodiments, therefore, those in need of therapy/treatment may include those already having the disease, disorder or condition and/or those prone to, or at risk of developing, the disease, disorder or condition and/or those in whom the disease, disorder or condition is to be prevented.

In certain embodiments, the amylin receptor antagonist of the present disclosure is effective for reducing cyclic AMP (cAMP) signal production in a cell. For example, administration of a therapeutically effective amount of the amylin receptor antagonist may cause a reduction in cAMP signal production in a cell as compared to a cell that has not been administered the amylin receptor antagonist.

In certain embodiments, compounds of the present disclosure produce a neuroprotective effect against amylin and/or amyloid-beta protein induced neurotoxicity. For example, in some cases, administration of a compound of the present disclosure is therapeutically effective for protecting neurons against the neurotoxic effect of amyloid-beta protein. In some cases, administration of a compound of the present disclosure is therapeutically effective for protecting neurons against the neurotoxic effect of amylin.

Methods of the present disclosure include administering to a subject in need thereof, a therapeutically effective amount of an amylin receptor antagonist. In certain embodiments, the amylin receptor antagonist is a non-peptidic compound. Non-peptidic compounds according to the present disclosure do not contain as part of their chemical structure a peptide or peptide derivative (e.g., modified peptide).

Formula (I)

In certain embodiments, the non-peptidic amylin receptor antagonist is a compound of formula (I):

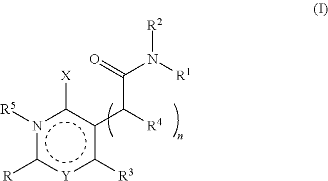

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² comprise a heterocycle, substituted heterocycle, fused-heterocycle or substituted fused-heterocycle;

R³ is selected from the group consisting of C₁-C₆-alkyl, substituted C₁-C₆-alkyl, C₃-C₆-cycloalkyl, substituted C₃-C₆-cycloalkyl, phenyl, and substituted phenyl;

each R⁴ is independently selected from the group consisting of —H and —CH₃;

R⁵ is present or absent, and if present is selected from the group consisting of —H and —CH₃;

each R⁶ is independently selected from the group consisting of —H, —CH₃, and —CH₂CH₃;

n is an integer from 1 to 3;

X is selected from the group consisting of ═O, ═NH, —N(R⁶)₂, and ═S;

Y is selected from the group consisting of —N═ and —CH═; and

Z is selected from the group consisting of ═O, ═NH, —N═, and ═S, wherein if Z is —N═, then Z together R¹ or R² comprises a heterocycle or substituted heterocycle;

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, R is selected from —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio. For example, in some embodiments, R can be —H. In some embodiments, R can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some embodiments, R can be C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some embodiments, R can be heterocyclyl or substituted heterocyclyl (e.g., unsubstituted or substituted pyrrolidinyl, imidazolidinyl, piperidinyl, piperazinyl, morpholinyl, and the like). In some embodiments, R can be —NHC(═O)R⁶. In some embodiments, R can be —N(R⁶)₂. In some embodiments, R can be —OR⁶. In some embodiments, R can be aryl or substituted aryl (e.g., unsubstituted or substituted phenyl). In some embodiments, R can be heteroaryl or substituted heteroaryl (e.g., unsubstituted or substituted pyrrolyl, pyrazolyl, imidazolyl, pyridinyl, pyrimidinyl, and the like). In some embodiments, R can be alkylthio (e.g., —S—(C₁-C₆-alkyl), such as —S-methyl, —S-ethyl, —S-propyl, —S-butyl, —S-pentyl, or —S-hexyl).

In certain embodiments, R¹ and R² are each independently selected from —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle.

For example, R¹ can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R¹ is —H. In some instances, R¹ is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R¹ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R¹ is heterocyclyl or substituted heterocyclyl (e.g., unsubstituted or substituted pyrrolidinyl, imidazolidinyl, piperidinyl, piperazinyl, morpholinyl, and the like). In some instances, R¹ is aryl or substituted aryl (e.g., unsubstituted or substituted phenyl). In some instances, R¹ is heteroaryl or substituted heteroaryl (e.g., unsubstituted or substituted pyrrolyl, pyrazolyl, imidazolyl, pyridinyl, pyrimidinyl, and the like). In some instances, R¹ is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In in some instances, R² can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R² is —H. In some instances, R² is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R² is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R² is heterocyclyl or substituted heterocyclyl (e.g., unsubstituted or substituted pyrrolidinyl, imidazolidinyl, piperidinyl, piperazinyl, morpholinyl, and the like). In some instances, R² is aryl or substituted aryl (e.g., unsubstituted or substituted phenyl). In some instances, R² is heteroaryl or substituted heteroaryl (e.g., unsubstituted or substituted pyrrolyl, pyrazolyl, imidazolyl, pyridinyl, pyrimidinyl, and the like). In some instances, R² is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In certain embodiments, R³ is selected from C₁-C₆-alkyl, substituted C₁-C₆-alkyl, C₃-C₆-cycloalkyl, substituted C₃-C₆-cycloalkyl, phenyl, and substituted phenyl. For example, R³ can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R³ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R³ is phenyl or substituted phenyl.

In certain embodiments, each R⁴ is independently selected from —H and —CH₃. In some instances, R⁴ is —H. In some instances, R⁴ is —CH₃.

In certain embodiments, R⁵ is present or absent, and if present is selected from —H and —CH₃. For example, R⁵ is present or absent depending on the number of bonds already present on the nitrogen to which R⁵ is attached. In some cases, the nitrogen to which R⁵ is attached has two bonds to the ring the nitrogen is incorporated into, and thus R⁵ is present. In other cases, the nitrogen to which R⁵ is attached has three bonds to the ring the nitrogen is incorporated into, and thus R⁵ is absent. When R⁵ is present, R⁵ can be —H or —CH₃. In some cases, R⁵ is —H. In some cases, R⁵ is —CH₃.

In certain embodiments, each R⁶ is independently selected from —H, —CH₃, and —CH₂CH₃. In some instances, R⁶ is —H. In some instances, R⁶ is —CH₃. In some instances, R⁶ is —CH₂CH₃.

In certain embodiments, n is an integer from 1 to 3. For example, n can be 1, 2 or 3.

In certain embodiments, X is selected from ═O, ═NH, —N(R⁶)₂, and ═S. In some cases, X is ═O. In some cases, X is ═NH. In some cases, X is —N(R⁶)₂. In some cases, X is ═S.

In certain embodiments, Y is selected from the group consisting of —N═ and —CH═. In some cases, Y is —N═. In some cases, Y is —CH═.

In certain embodiments, Z is selected from ═O, ═NH, —N═, and ═S, where if Z is —N═, then Z together R¹ or R² comprises a heterocycle or substituted heterocycle. In some instances, Z is ═O. In some instances, Z is ═NH. In some instances, Z is ═S. In some instances, Z is —N═. If Z is —N═, then Z together R¹ or R² comprises a heterocycle or substituted heterocycle (e.g., 1H-benzo[d]imidazole, and the like).

Formula (II)

In certain embodiments, the non-peptidic amylin receptor antagonist is a compound of formula (II):

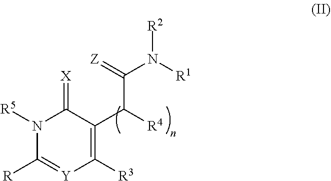

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle;

R³ is selected from the group consisting of C₁-C₆-alkyl, substituted C₁-C₆-alkyl, C₃-C₆-cycloalkyl, substituted C₃-C₆-cycloalkyl, phenyl, and substituted phenyl;

each R⁴ is independently selected from the group consisting of —H and —CH₃;

R⁵ is selected from the group consisting of —H and —CH₃;

each R⁶ is independently selected from the group consisting of —H, —CH₃, and —CH₂CH₃;

n is an integer from 1 to 3;

X is selected from the group consisting of ═O, ═NH, and ═S;

Y is selected from the group consisting of —N═ and —CH═; and

Z is selected from the group consisting of ═O, ═NH, and ═S;

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, R is selected from —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio. For example, in some embodiments, R can be —H. In some embodiments, R can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some embodiments, R can be C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some embodiments, R can be heterocyclyl or substituted heterocyclyl (e.g., unsubstituted or substituted pyrrolidinyl, imidazolidinyl, piperidinyl, piperazinyl, morpholinyl, and the like). In some embodiments, R can be —NHC(═O)R⁶. In some embodiments, R can be —N(R⁶)₂. In some embodiments, R can be —OR⁶. In some embodiments, R can be aryl or substituted aryl (e.g., unsubstituted or substituted phenyl). In some embodiments, R can be heteroaryl or substituted heteroaryl (e.g., unsubstituted or substituted pyrrolyl, pyrazolyl, imidazolyl, pyridinyl, pyrimidinyl, and the like). In some embodiments, R can be alkylthio (e.g., —S—(C₁-C₆-alkyl), such as —S-methyl, —S-ethyl, —S-propyl, —S-butyl, —S-pentyl, or —S-hexyl).

In certain embodiments, R¹ and R² are each independently selected from —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle.

For example, R¹ can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R¹ is —H. In some instances, R¹ is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R¹ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R¹ is heterocyclyl or substituted heterocyclyl (e.g., unsubstituted or substituted pyrrolidinyl, imidazolidinyl, piperidinyl, piperazinyl, morpholinyl, and the like). In some instances, R¹ is aryl or substituted aryl (e.g., unsubstituted or substituted phenyl). In some instances, R¹ is heteroaryl or substituted heteroaryl (e.g., unsubstituted or substituted pyrrolyl, pyrazolyl, imidazolyl, pyridinyl, pyrimidinyl, and the like). In some instances, R¹ is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In in some instances, R² can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R² is —H. In some instances, R² is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R² is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R² is heterocyclyl or substituted heterocyclyl (e.g., unsubstituted or substituted pyrrolidinyl, imidazolidinyl, piperidinyl, piperazinyl, morpholinyl, and the like). In some instances, R² is aryl or substituted aryl (e.g., unsubstituted or substituted phenyl). In some instances, R² is heteroaryl or substituted heteroaryl (e.g., unsubstituted or substituted pyrrolyl, pyrazolyl, imidazolyl, pyridinyl, pyrimidinyl, and the like). In some instances, R² is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In certain embodiments, R³ is selected from C₁-C₆-alkyl, substituted C₁-C₆-alkyl, C₃-C₆-cycloalkyl, substituted C₃-C₆-cycloalkyl, phenyl, and substituted phenyl. For example, R³ can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R³ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R³ is phenyl or substituted phenyl.

In certain embodiments, each R⁴ is independently selected from —H and —CH₃. In some instances, R⁴ is —H. In some instances, R⁴ is —CH₃.

In certain embodiments, R⁵ is selected from —H and —CH₃. In some cases, R⁵ is —H. In some cases, R⁵ is —CH₃.

In certain embodiments, each R⁶ is independently selected from —H, —CH₃, and —CH₂CH₃. In some instances, R⁶ is —H. In some instances, R⁶ is —CH₃. In some instances, R⁶ is —CH₂CH₃.

In certain embodiments, n is an integer from 1 to 3. For example, n can be 1, 2 or 3.

In certain embodiments, X is selected from ═O, ═NH, and ═S. In some cases, X is ═O. In some cases, X is ═NH. In some cases, X is ═S.

In certain embodiments, Y is selected from the group consisting of —N═ and —CH═. In some cases, Y is —N═. In some cases, Y is —CH═.

In certain embodiments, Z is selected from ═O, ═NH, and ═S. In some instances, Z is ═O. In some instances, Z is ═NH. In some instances, Z is ═S.

Formula (III)

In certain embodiments, the non-peptidic amylin receptor antagonist is a compound of formula (III):

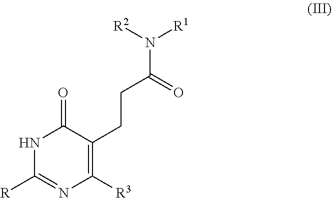

wherein:

R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;

R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle; and

R³ is selected from the group consisting of C₁-C₆-alkyl, substituted C₁-C₆-alkyl, C₃-C₆-cycloalkyl, substituted C₃-C₆-cycloalkyl, phenyl, and substituted phenyl;

or an enantiomer, a mixture of enantiomers, a mixture of two or more diastereomers, a tautomer, a mixture of two or more tautomers, or an isotopic variant thereof;

or a pharmaceutically acceptable salt, solvate, or hydrate thereof.

In certain embodiments, R is selected from —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio. For example, in some embodiments, R can be —H. In some embodiments, R can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some embodiments, R can be C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some embodiments, R can be heterocyclyl or substituted heterocyclyl (e.g., unsubstituted or substituted pyrrolidinyl, imidazolidinyl, piperidinyl, piperazinyl, morpholinyl, and the like). In some embodiments, R can be —NHC(═O)R⁶. In some embodiments, R can be —N(R⁶)₂. In some embodiments, R can be —OR⁶. In some embodiments, R can be aryl or substituted aryl (e.g., unsubstituted or substituted phenyl). In some embodiments, R can be heteroaryl or substituted heteroaryl (e.g., unsubstituted or substituted pyrrolyl, pyrazolyl, imidazolyl, pyridinyl, pyrimidinyl, and the like). In some embodiments, R can be alkylthio (e.g., —S—(C₁-C₆-alkyl), such as —S-methyl, —S-ethyl, —S-propyl, —S-butyl, —S-pentyl, or —S-hexyl).

In certain embodiments, R¹ and R² are each independently selected from —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle.

For example, R¹ can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R¹ is —H. In some instances, R¹ is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R¹ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R¹ is heterocyclyl or substituted heterocyclyl (e.g., unsubstituted or substituted pyrrolidinyl, imidazolidinyl, piperidinyl, piperazinyl, morpholinyl, and the like). In some instances, R¹ is aryl or substituted aryl (e.g., unsubstituted or substituted phenyl). In some instances, R¹ is heteroaryl or substituted heteroaryl (e.g., unsubstituted or substituted pyrrolyl, pyrazolyl, imidazolyl, pyridinyl, pyrimidinyl, and the like). In some instances, R¹ is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In in some instances, R² can be —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, or substituted fused-heterocycle. In some instances, R² is —H. In some instances, R² is C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R² is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R² is heterocyclyl or substituted heterocyclyl (e.g., unsubstituted or substituted pyrrolidinyl, imidazolidinyl, piperidinyl, piperazinyl, morpholinyl, and the like). In some instances, R² is aryl or substituted aryl (e.g., unsubstituted or substituted phenyl). In some instances, R² is heteroaryl or substituted heteroaryl (e.g., unsubstituted or substituted pyrrolyl, pyrazolyl, imidazolyl, pyridinyl, pyrimidinyl, and the like). In some instances, R² is fused-heterocycle or substituted fused-heterocycle (e.g., unsubstituted or substituted 3,4-dihydro-2H-benzo[b][1,4]oxazine, 2H-benzo[b][1,4]oxazin-3(4H)-one, benzo[d][1,3]dioxole, 3,4-dihydroquinoxalin-2(1H)-one, 1,2,3,4-tetrahydroquinoxaline, spiro[benzo[d][1,3]dioxole-2,1′-cyclohexane], 1,2,3,4-tetrahydroisoquinoline, 1,2,3,4-tetrahydroquinoline, indoline, 1H-benzo[d]imidazole, and the like).

In certain embodiments, R³ is selected from C₁-C₆-alkyl, substituted C₁-C₆-alkyl, C₃-C₆-cycloalkyl, substituted C₃-C₆-cycloalkyl, phenyl, and substituted phenyl. For example, R³ can be C₁-C₆-alkyl (e.g., methyl, ethyl, propyl, butyl, pentyl, or hexyl) or substituted C₁-C₆-alkyl (e.g., substituted methyl, substituted ethyl, substituted propyl, substituted butyl, substituted pentyl, or substituted hexyl). In some instances, R³ is C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some instances, R³ is phenyl or substituted phenyl.

Compounds of the present disclosure (e.g., compounds of formulae (I), (II) and (III) as described herein) also include an enantiomer, a mixture of enantiomers, a mixture of two or more diastereomers, a tautomer, a mixture of two or more tautomers, or an isotopic variant thereof.

In addition, compounds of the present disclosure (e.g., compounds of formulae (I), (II) and (III) as described herein) also include a pharmaceutically acceptable salt, solvate, or hydrate thereof.

In certain embodiments, compounds of the present disclosure (e.g., compounds that find use in the methods of the present disclosure) include compounds selected from:

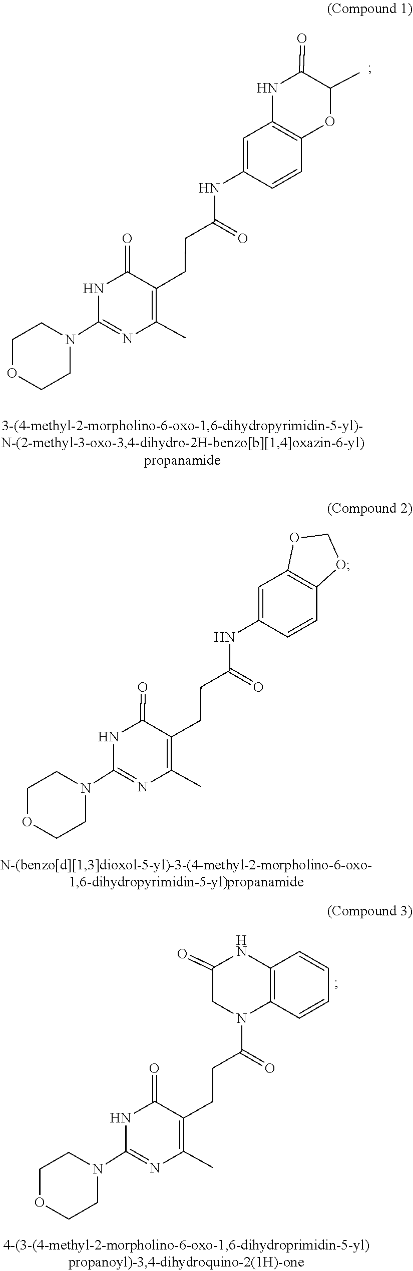 [SMILES: Cc1nc(N2CCOCC2)[nH]c(=O)c1CCC(=O)Nc1ccc2c(c1)NC(=O)C(C)O2 ; Cc1nc(N2CCOCC2)[nH]c(=O)c1CCC(=O)Nc1ccc2c(c1)OCO2 ; Cc1nc(N2CCOCC2)[nH]c(=O)c1CCC(=O)N1CC(=O)Nc2ccccc21]

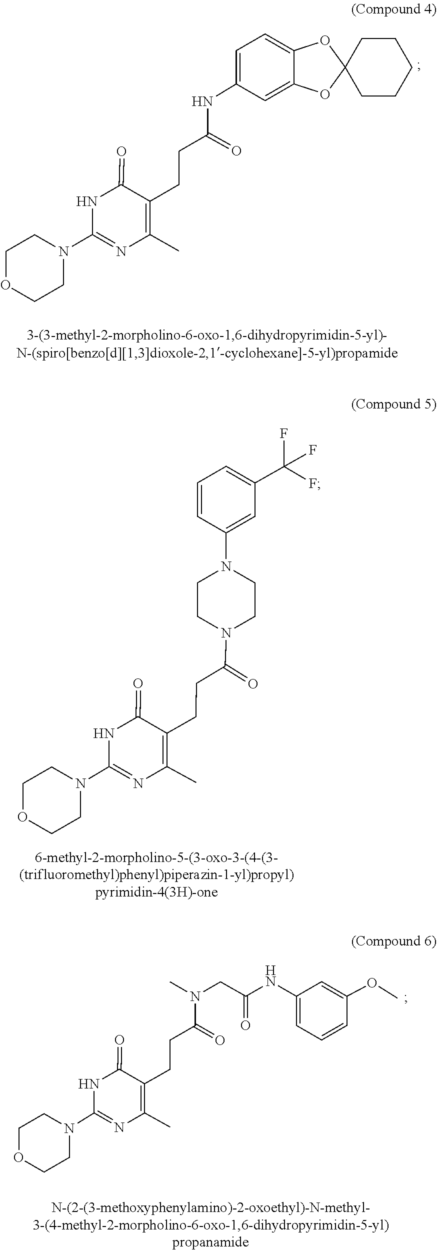 [SMILES: Cc1nc(N2CCOCC2)[nH]c(=O)c1CCC(=O)Nc1ccc2c(c1)OC1(CCCCC1)O2 ; Cc1nc(N2CCOCC2)[nH]c(=O)c1CCC(=O)N1CCN(c2cccc(C(F)(F)F)c2)CC1 ; COc1cccc(NC(=O)CN(C)C(=O)CCc2c(C)nc(N3CCOCC3)[nH]c2=O)c1]

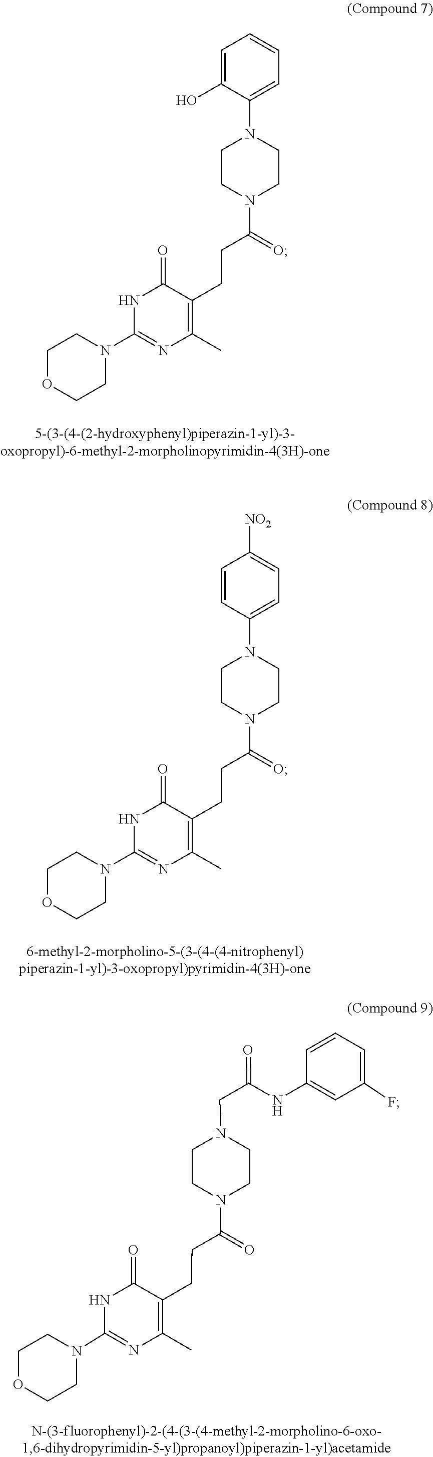 [SMILES: Cc1nc(N2CCOCC2)[nH]c(=O)c1CCC(=O)N1CCN(c2ccccc2O)CC1 ; Cc1nc(N2CCOCC2)[nH]c(=O)c1CCC(=O)N1CCN(c2ccc([N+](=O)[O-])cc2)CC1 ; Cc1nc(N2CCOCC2)[nH]c(=O)c1CCC(=O)N1CCN(CC(=O)Nc2cccc(F)c2)CC1]

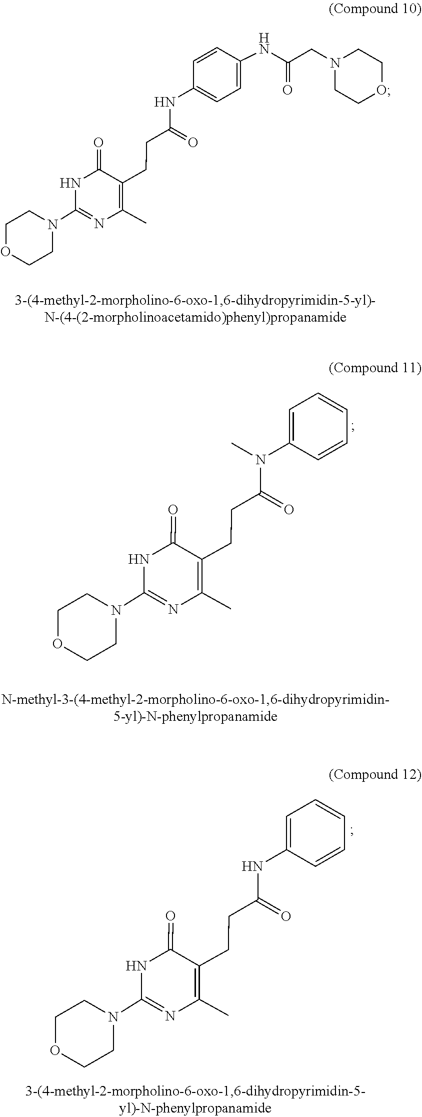 [SMILES: Cc1nc(N2CCOCC2)[nH]c(=O)c1CCC(=O)Nc1ccc(NC(=O)CN2CCOCC2)cc1 ; Cc1nc(N2CCOCC2)[nH]c(=O)c1CCC(=O)N(C)c1ccccc1 ; Cc1nc(N2CCOCC2)[nH]c(=O)c1CCC(=O)Nc1ccccc1]

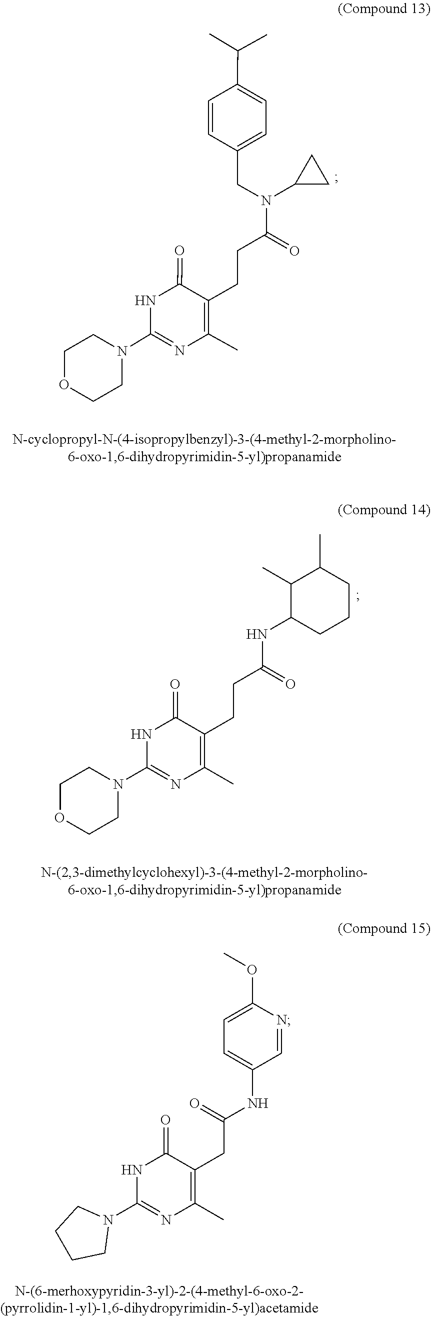 [SMILES: Cc1nc(N2CCOCC2)[nH]c(=O)c1CCC(=O)N(Cc1ccc(C(C)C)cc1)C1CC1 ; Cc1nc(N2CCOCC2)[nH]c(=O)c1CCC(=O)NC1CCCC(C)C1C ; COc1ccc(NC(=O)Cc2c(C)nc(N3CCCC3)[nH]c2=O)cn1]

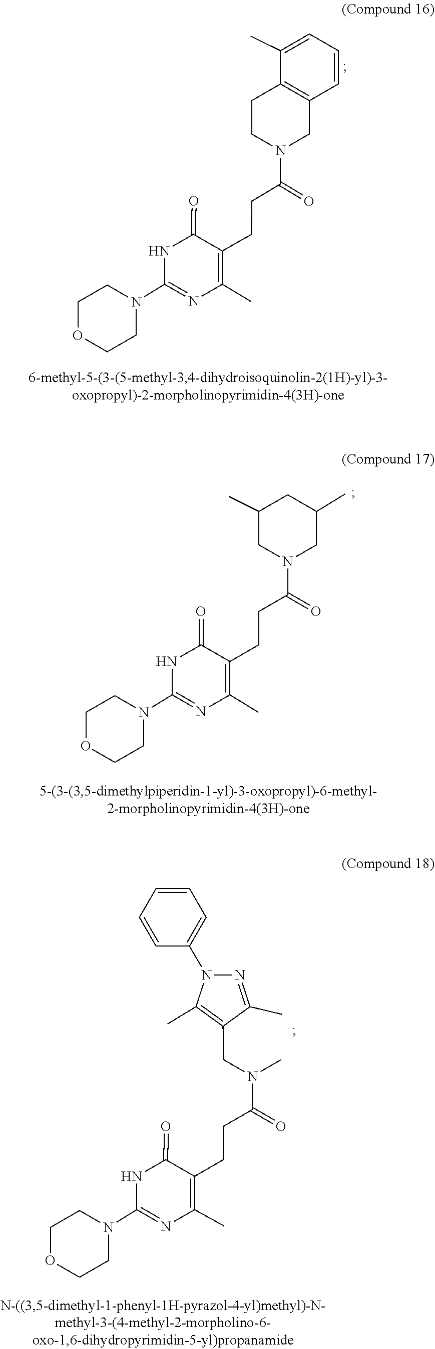 [SMILES: Cc1cccc2c1CCN(C(=O)CCc1c(C)nc(N3CCOCC3)[nH]c1=O)C2 ; Cc1nc(N2CCOCC2)[nH]c(=O)c1CCC(=O)N1CC(C)CC(C)C1 ; Cc1nn(-c2ccccc2)c(C)c1CN(C)C(=O)CCc1c(C)nc(N2CCOCC2)[nH]c1=O]

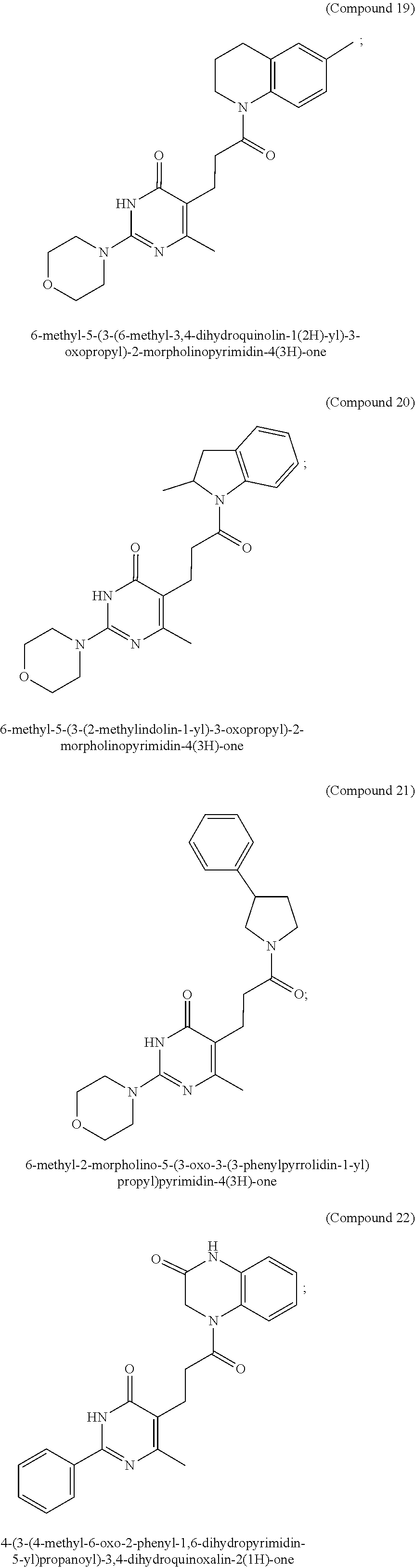 [SMILES: Cc1ccc2c(c1)CCCN2C(=O)CCc1c(C)nc(N2CCOCC2)[nH]c1=O ; Cc1nc(N2CCOCC2)[nH]c(=O)c1CCC(=O)N1c2ccccc2CC1C ; Cc1nc(N2CCOCC2)[nH]c(=O)c1CCC(=O)N1CCC(c2ccccc2)C1 ; Cc1nc(-c2ccccc2)[nH]c(=O)c1CCC(=O)N1CC(=O)Nc2ccccc21]

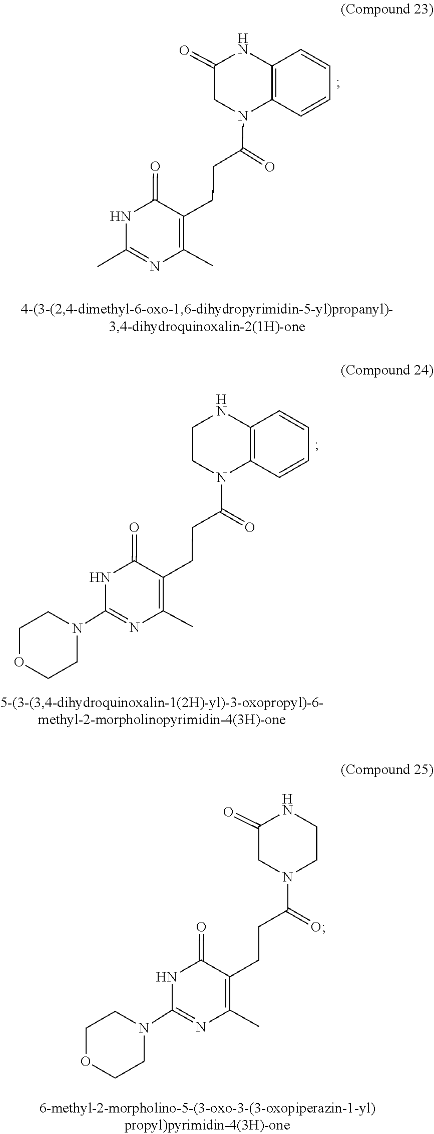 [SMILES: Cc1nc(C)c(CCC(=O)N2CC(=O)Nc3ccccc32)c(=O)[nH]1 ; Cc1nc(N2CCOCC2)[nH]c(=O)c1CCC(=O)N1CCNc2ccccc21 ; Cc1nc(N2CCOCC2)[nH]c(=O)c1CCC(=O)N1CCNC(=O)C1]

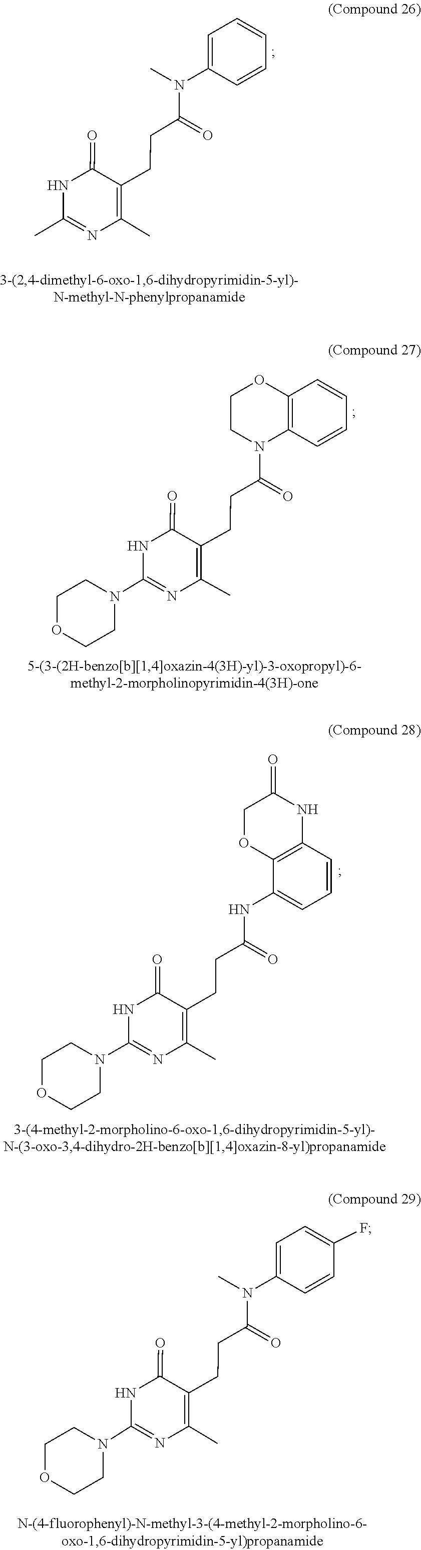 [SMILES: Cc1nc(C)c(CCC(=O)N(C)c2ccccc2)c(=O)[nH]1 ; Cc1nc(N2CCOCC2)[nH]c(=O)c1CCC(=O)N1CCOc2ccccc21 ; Cc1nc(N2CCOCC2)[nH]c(=O)c1CCC(=O)Nc1cccc2c1OCC(=O)N2 ; Cc1nc(N2CCOCC2)[nH]c(=O)c1CCC(=O)N(C)c1ccc(F)cc1]

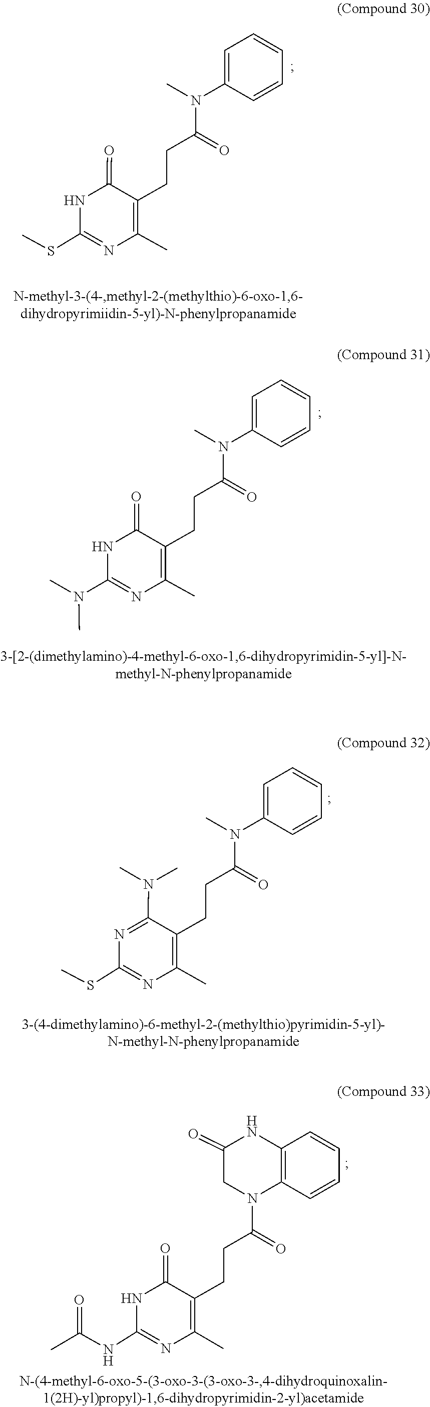 [SMILES: CSc1nc(C)c(CCC(=O)N(C)c2ccccc2)c(=O)[nH]1 ; Cc1nc(N(C)C)[nH]c(=O)c1CCC(=O)N(C)c1ccccc1 ; CSc1nc(C)c(CCC(=O)N(C)c2ccccc2)c(N(C)C)n1 ; CC(=O)Nc1nc(C)c(CCC(=O)N2CC(=O)Nc3ccccc32)c(=O)[nH]1]

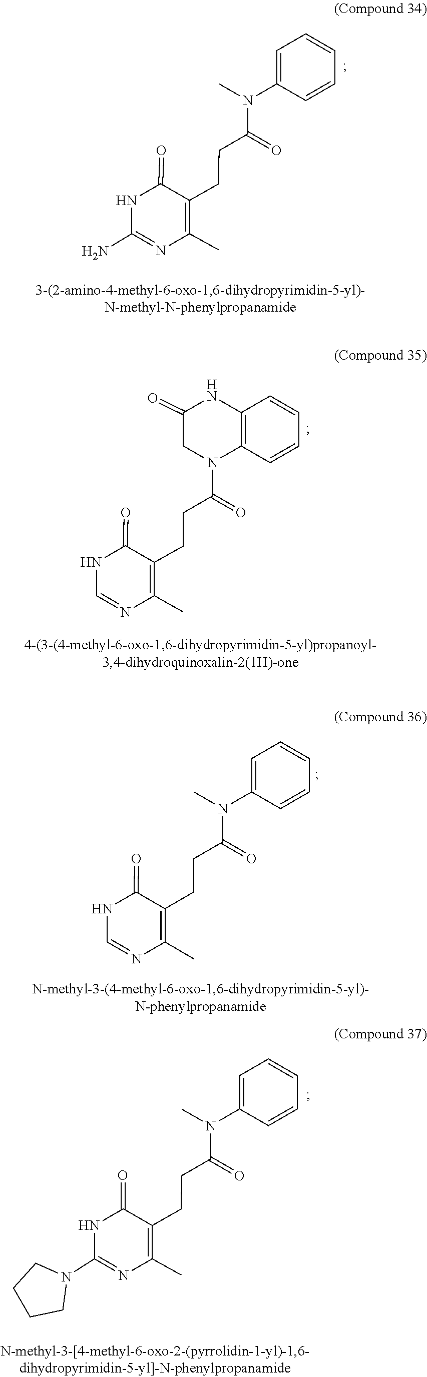 [SMILES: Cc1nc(N)[nH]c(=O)c1CCC(=O)N(C)c1ccccc1 ; Cc1nc[nH]c(=O)c1CCC(=O)N1CC(=O)Nc2ccccc21 ; Cc1nc[nH]c(=O)c1CCC(=O)N(C)c1ccccc1 ; Cc1nc(N2CCCC2)[nH]c(=O)c1CCC(=O)N(C)c1ccccc1]

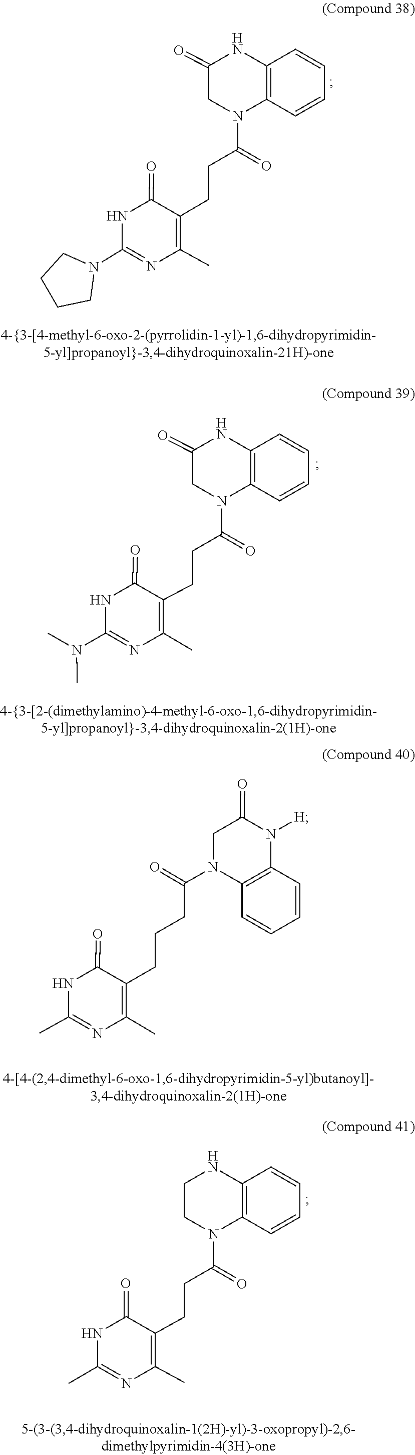

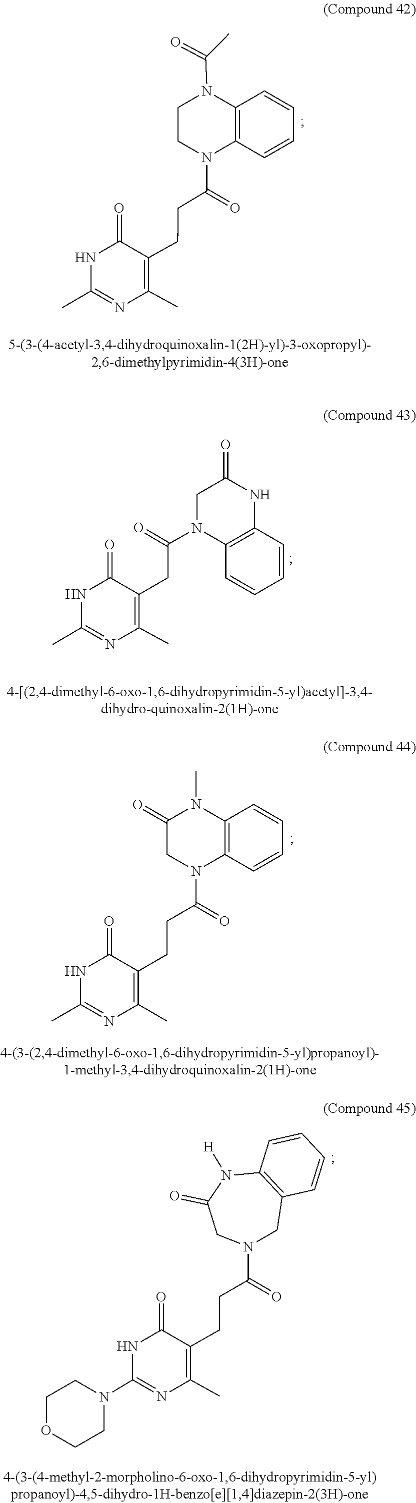 [SMILES: CC(=O)N1CCN(C(=O)CCc2c(C)nc(C)[nH]c2=O)c2ccccc21 ; Cc1nc(C)c(CC(=O)N2CC(=O)Nc3ccccc32)c(=O)[nH]1 ; Cc1nc(C)c(CCC(=O)N2CC(=O)N(C)c3ccccc32)c(=O)[nH]1 ; Cc1nc(N2CCOCC2)[nH]c(=O)c1CCC(=O)N1CC(=O)Nc2ccccc2C1]

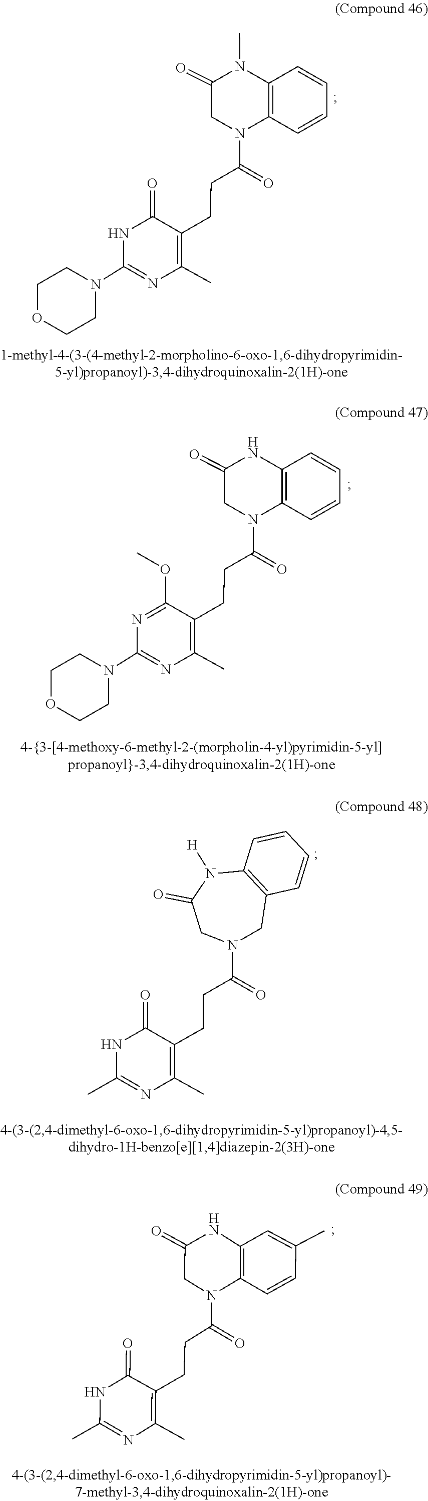

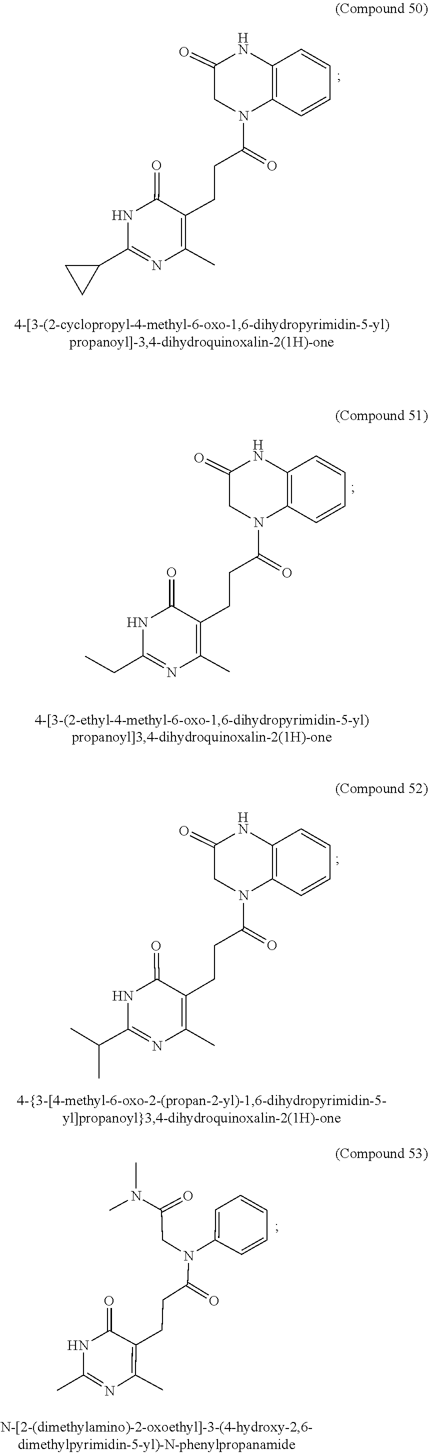 [SMILES: Cc1nc(C2CC2)[nH]c(=O)c1CCC(=O)N1CC(=O)Nc2ccccc21 ; CCc1nc(C)c(CCC(=O)N2CC(=O)Nc3ccccc32)c(=O)[nH]1 ; Cc1nc(C(C)C)[nH]c(=O)c1CCC(=O)N1CC(=O)Nc2ccccc21 ; Cc1nc(C)c(CCC(=O)N(CC(=O)N(C)C)c2ccccc2)c(=O)[nH]1]

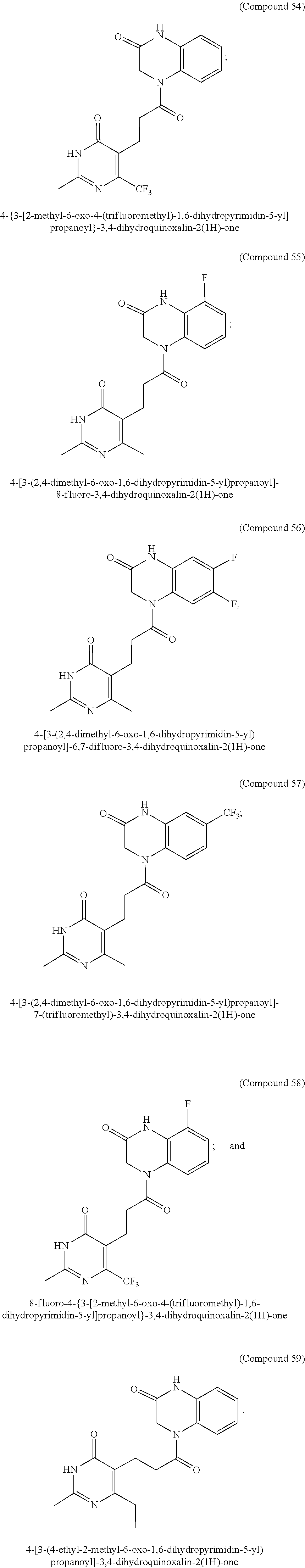 [SMILES: Cc1nc(C(F)(F)F)c(CCC(=O)N2CC(=O)Nc3ccccc32)c(=O)[nH]1 ; Cc1nc(C)c(CCC(=O)N2CC(=O)Nc3c(F)cccc32)c(=O)[nH]1 ; Cc1nc(C)c(CCC(=O)N2CC(=O)Nc3cc(F)c(F)cc32)c(=O)[nH]1 ; Cc1nc(C)c(CCC(=O)N2CC(=O)Nc3cc(C(F)(F)F)ccc32)c(=O)[nH]1 ; Cc1nc(C(F)(F)F)c(CCC(=O)N2CC(=O)Nc3c(F)cccc32)c(=O)[nH]1 ; CCc1nc(C)[nH]c(=O)c1CCC(=O)N1CC(=O)Nc2ccccc21]

Formula (IV)

In certain embodiments, the non-peptidic amylin receptor antagonist is a compound of formula (IV):

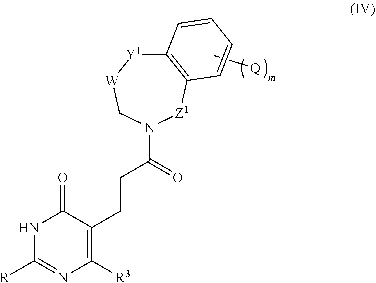

wherein:

R is selected from the group consisting of —H, C₁-C₃-alkyl, substituted C₁-C₃-alkyl, C₃-C₆-cycloalkyl, substituted C₃-C₆-cycloalkyl, heterocyclyl, substituted heterocyclyl, aryl, substituted aryl, —NHC(═O)R⁹, —N(R⁹)₂, —OR⁹, and —SR⁹;

R³ is selected from the group consisting of C₁-C₆-alkyl, C₃-C₆-cycloalkyl, and —CF₃;

m is 0, 1 or 2;

W is selected from the group consisting of —C(═O)— and —CH₂—;

each Q is independently selected from the group consisting of —F, —Cl, —CN, —CF₃ and C₁-C₃-alkyl;

Y¹ is selected from the group consisting of —NH—, —N(CH₃)—, —N(CH₂CH₃)— and —N(cyclopropyl)-;

each R⁹ is independently selected from the group consisting of —H, —CH₃, —CH₂CH₃ and cyclopropyl; and

Z¹ is absent or is —CH₂—;

or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof;

with the proviso that the compound is not: 4-[3-[1,6-dihydro-4-methyl-2-(4-morpholinyl)-6-oxo-5-pyrimidinyl]-1-oxopropyl]-3,4-dihydro-2(1H)-quinoxalinone, 6-methyl-2-(4-morpholinyl)-5-[3-oxo-3-(1,2,3,5-tetrahydro-1-methyl-4H-1,4-benzodiazepin-4-yl)propyl]-4(3H)-pyrimidinone, or 5-[3-(3,4-dihydro-4-methyl-1(2H)-quinoxalinyl)-3-oxopropyl]-6-methyl-2-(4-morpholinyl)-4(3H)-pyrimidinone.

In certain embodiments, R is selected from —H, C₁-C₃-alkyl, substituted C₁-C₃-alkyl, C₃-C₆-cycloalkyl, substituted C₃-C₆-cycloalkyl, heterocyclyl, substituted heterocyclyl, aryl, substituted aryl, —N(═O)R⁹, —N(R⁹)₂, —OR⁹, and —SR⁹. In certain embodiments, R is selected from —H, C₁-C₃-alkyl, C₃-C₆-cycloalkyl, heterocyclyl, phenyl, —N(═O)R⁹, —N(R⁹)₂, —OR⁹, and —SR⁹. For example, in some embodiments, R can be —H. In some embodiments, R can be C₁-C₃-alkyl (e.g., methyl, ethyl, or propyl) or substituted C₁-C₃-alkyl (e.g., substituted methyl, substituted ethyl, or substituted propyl). In some embodiments, R can be C₃-C₆-cycloalkyl (e.g., cyclopropyl, cyclobutyl, cyclopentyl, or cyclohexyl) or substituted C₃-C₆-cycloalkyl (e.g., substituted cyclopropyl, substituted cyclobutyl, substituted cyclopentyl, or substituted cyclohexyl). In some embodiments, R can be heterocyclyl or substituted heterocyclyl (e.g., unsubstituted or substituted azetidinyl, pyrrolidinyl, imidazolidinyl, piperidinyl, piperazinyl, morpholinyl, and the like). In some embodiments, R can be aryl or substituted aryl (e.g., unsubstituted or substituted phenyl). In some embodiments, R can be —NHC(═O)R⁹. For example, R can be —NHC(═O)CH₃ or —NHC(═O)CH₂CH₃. In some embodiments, R can be —N(R⁹)₂. For example, R can be —NH₂, —N(CH₃)₂ or —N(CH₂CH₃)₂. In some embodiments, R can be —OR⁹. For example, R can be —OH, —OCH₃ or —OCH₂CH₃. In some embodiments, R can be —SR⁹. For example, R can be —SH, —SCH₃ or —SCH₂CH₃.

In certain embodiments, R³ is selected from C₁-C₆-alkyl, C₃-C₆-cycloalkyl, and —CF₃. For example, in some embodiments, R³ can be C₁-C₆-alkyl (e.g., methyl, ethyl, propyl, butyl, pentyl, or hexyl. In some embodiments, R³ can be C₃-C₆-cycloalkyl (e.g., cyclopropyl, cyclobutyl, cyclopentyl, or cyclohexyl). In some embodiments, R³ is CF₃.

In certain embodiments, m is 0, 1 or 2. In some instances, m is 0. In some instances, m is 1. In some instances, m is 2.

In certain embodiments, W is selected from —C(═O)— and —CH₂—. In some instances, W is —C(═O)—. In some instances, W is —CH₂—.

In certain embodiments, each Q is independently selected from —F, —Cl, —CN, —CF₃ and C₁-C₃-alkyl. In some instances, Q is halogen, such as —F, —Cl, or —Br. In some instances, Q is —CN. In some instances, Q is —CF₃. In some instances, Q can be C₁-C₃-alkyl (e.g., methyl, ethyl, or propyl) or substituted C₁-C₃-alkyl (e.g., substituted methyl, substituted ethyl, or substituted propyl).

In certain embodiments, Y¹ is selected from —NH—, —N(CH₃)—, —N(CH₂CH₃)— and —N(cyclopropyl)-. In some instances, Y¹ is —NH—. In some instances, Y¹ is —N(CH₃)—. In some instances, Y¹ is —N(CH₂CH₃)—. In some instances, Y¹ is —N(cyclopropyl)-.

In certain embodiments, each R⁹ is independently selected from —H, —CH₃, —CH₂CH₃ and cyclopropyl. In some instances, R⁹ is —H. In some instances, R⁹ is —CH₃. In some instances, R⁹ is —CH₂CH₃. In some instances, R⁹ is cyclopropyl.

In certain embodiments, Z¹ is absent or is —CH₂—. In some instances, Z¹ is absent. In some instances, Z¹ is —CH₂—.

Compounds of the present disclosure (e.g., compounds of formula (IV) as described herein) also include an enantiomer, a mixture of enantiomers, a mixture of two or more diastereomers, a tautomer, a mixture of two or more tautomers, or an isotopic variant thereof.

In addition, compounds of the present disclosure (e.g., compounds of formula (IV) as described herein) also include a pharmaceutically acceptable salt, solvate, or hydrate thereof.

In certain embodiments, compounds of the present disclosure (e.g., compounds that find use in the methods of the present disclosure) include compounds of formula (IV) selected from:

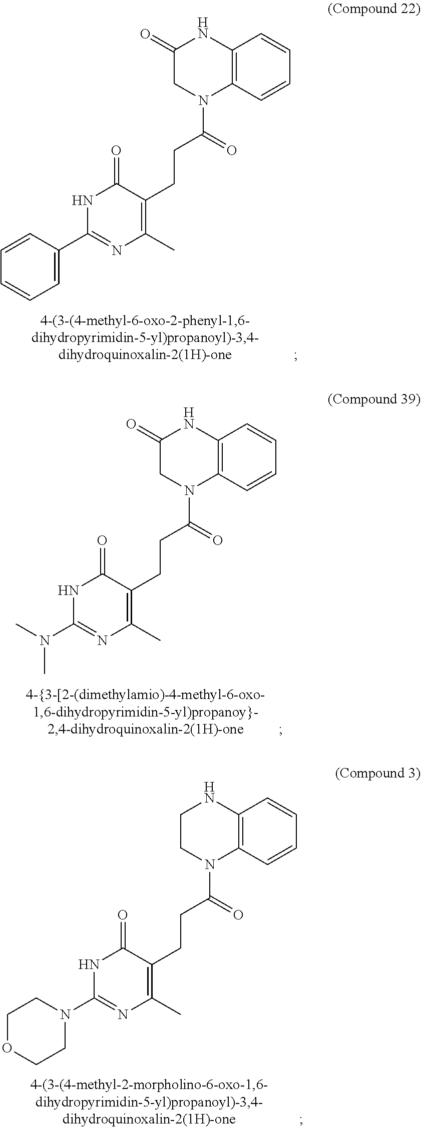 [SMILES: Cc1nc(-c2ccccc2)[nH]c(=O)c1CCC(=O)N1CC(=O)Nc2ccccc21 ; Cc1nc(N(C)C)[nH]c(=O)c1CCC(=O)N1CC(=O)Nc2ccccc21 ; Cc1nc(N2CCOCC2)[nH]c(=O)c1CCC(=O)N1CCNc2ccccc21]

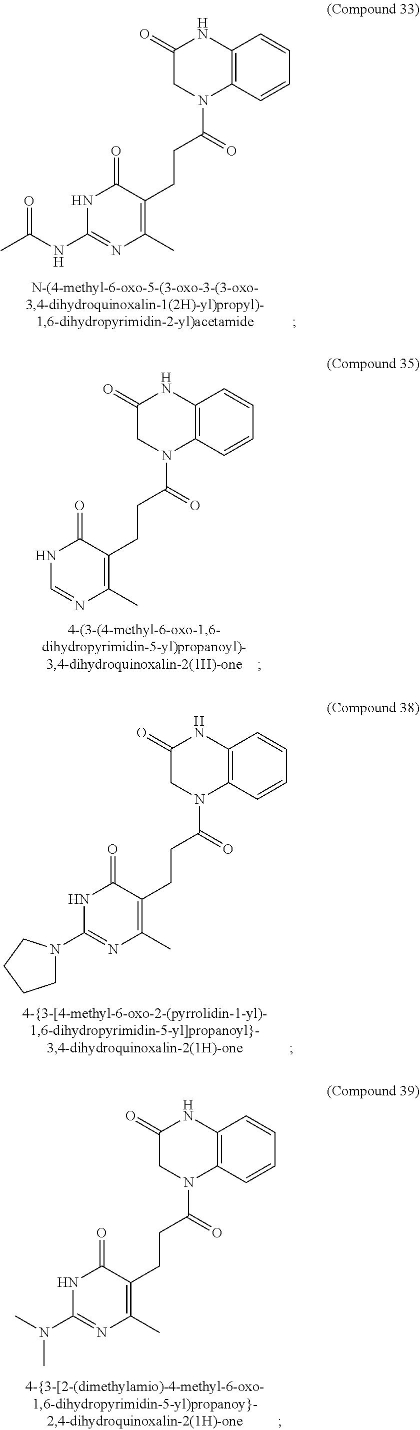 [SMILES: CC(=O)Nc1nc(C)c(CCC(=O)N2CC(=O)Nc3ccccc32)c(=O)[nH]1 ; Cc1nc[nH]c(=O)c1CCC(=O)N1CC(=O)Nc2ccccc21 ; Cc1nc(N2CCCC2)[nH]c(=O)c1CCC(=O)N1CC(=O)Nc2ccccc21 ; Cc1nc(N(C)C)[nH]c(=O)c1CCC(=O)N1CC(=O)Nc2ccccc21]

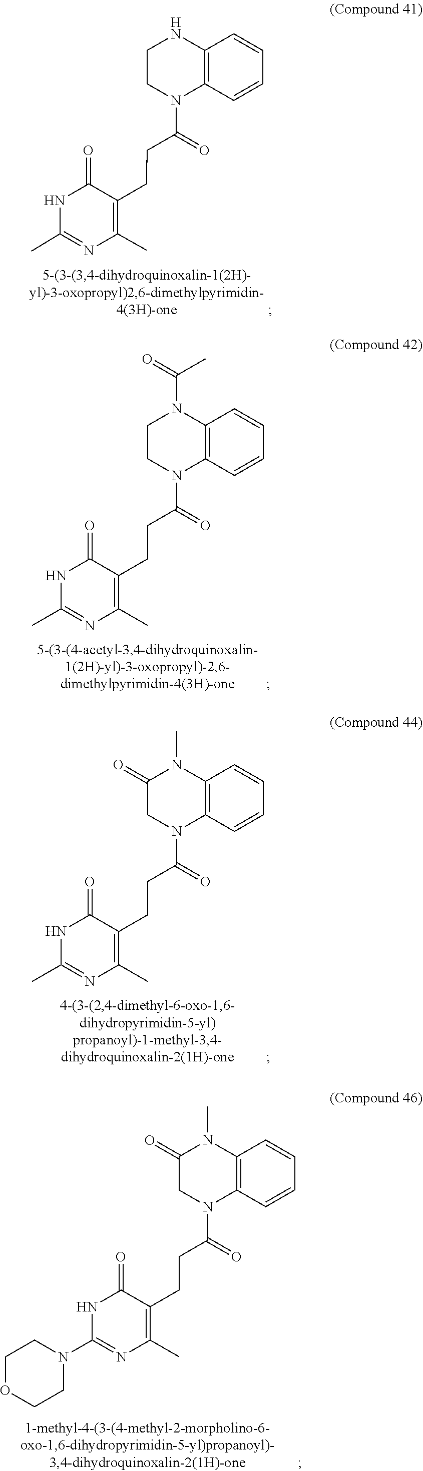 [SMILES: Cc1nc(C)c(CCC(=O)N2CCNc3ccccc32)c(=O)[nH]1 ; CC(=O)N1CCN(C(=O)CCc2c(C)nc(C)[nH]c2=O)c2ccccc21 ; Cc1nc(C)c(CCC(=O)N2CC(=O)N(C)c3ccccc32)c(=O)[nH]1 ; Cc1nc(N2CCOCC2)[nH]c(=O)c1CCC(=O)N1CC(=O)N(C)c2ccccc21]

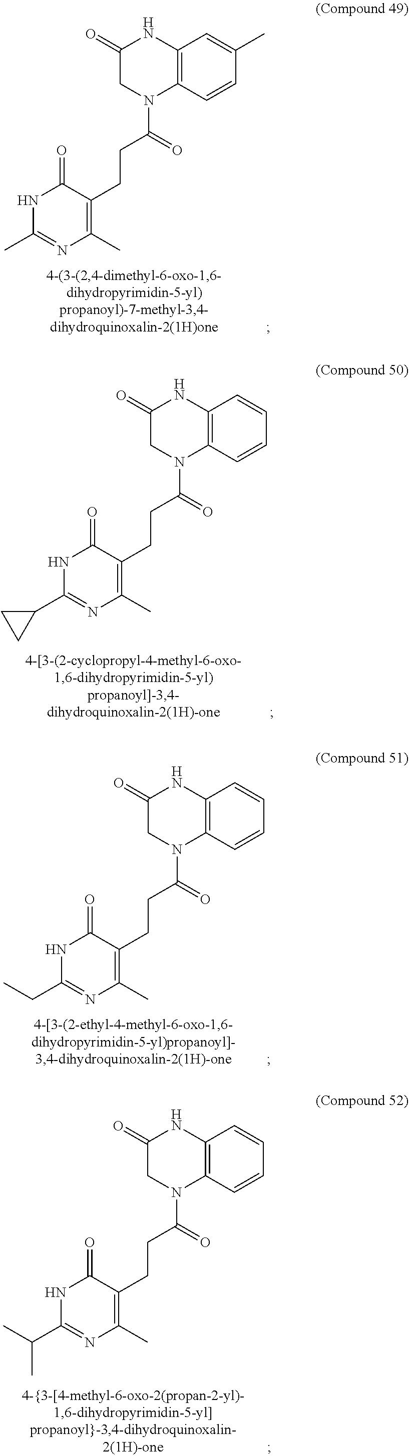

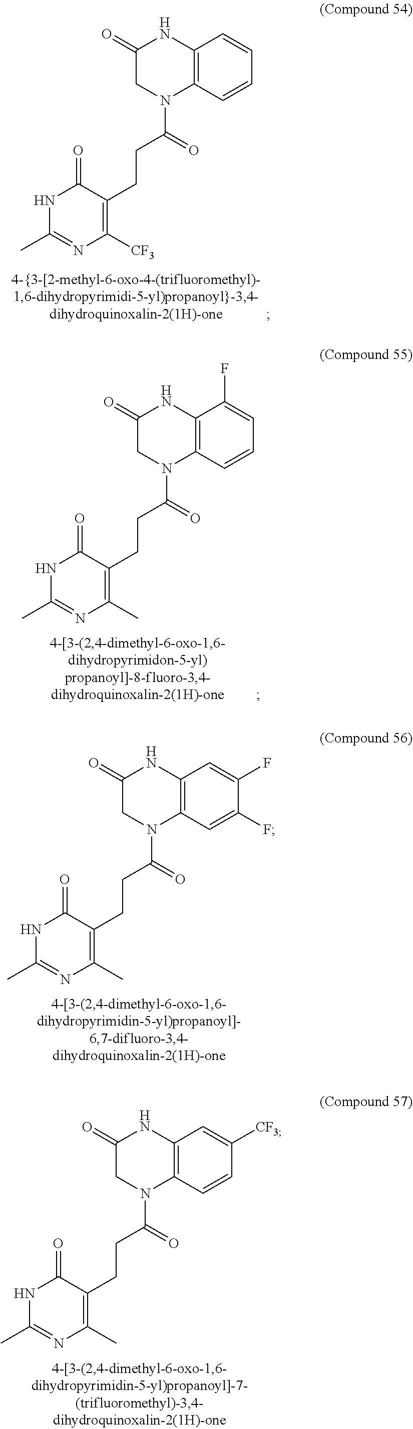 [SMILES: Cc1nc(C(F)(F)F)c(CCC(=O)N2CC(=O)Nc3ccccc32)c(=O)[nH]1 ; Cc1nc(C)c(CCC(=O)N2CC(=O)Nc3c(F)cccc32)c(=O)[nH]1 ; Cc1nc(C)c(CCC(=O)N2CC(=O)Nc3cc(F)c(F)cc32)c(=O)[nH]1 ; Cc1nc(C)c(CCC(=O)N2CC(=O)Nc3cc(C(F)(F)F)ccc32)c(=O)[nH]1]

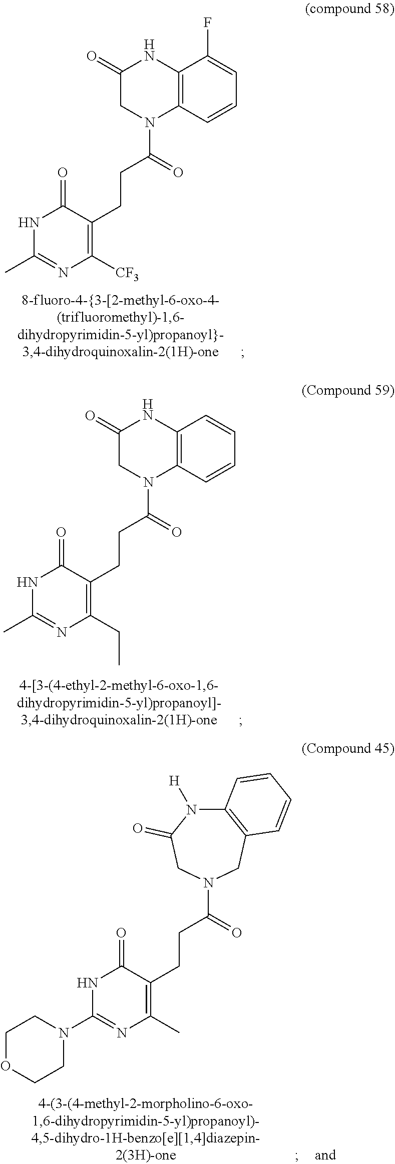 [SMILES: Cc1nc(C(F)(F)F)c(CCC(=O)N2CC(=O)Nc3c(F)cccc32)c(=O)[nH]1 ; CCc1nc(C)[nH]c(=O)c1CCC(=O)N1CC(=O)Nc2ccccc21 ; Cc1nc(N2CCOCC2)[nH]c(=O)c1CCC(=O)N1CC(=O)Nc2ccccc2C1]

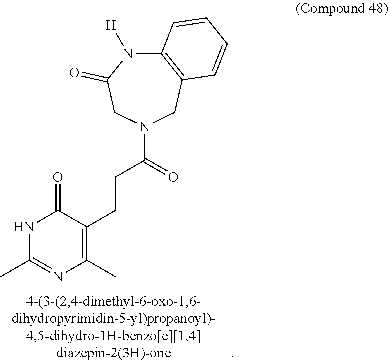 [SMILES: Cc1nc(C)c(CCC(=O)N2CC(=O)Nc3ccccc3C2)c(=O)[nH]1]

In certain embodiments, the compound of formula (IV) does not include 4-[3-[1,6-dihydro-4-methyl-2-(4-morpholinyl)-6-oxo-5-pyrimidinyl]-1-oxopropyl]-3,4-dihydro-2(1H)-quinoxalinone, 6-methyl-2-(4-morpholinyl)-5-[3-oxo-3-(1,2,3,5-tetrahydro-1-methyl-4H-1,4-benzodiazepin-4-yl)propyl]-4(3H)-pyrimidinone, or 5-[3-(3,4-dihydro-4-methyl-1(2H)-quinoxalinyl)-3-oxopropyl]-6-methyl-2-(4-morpholinyl)-4(3H)-pyrimidinone.

The compounds of the present disclosure find use in treatment of a condition or disease in a subject that is amenable to treatment by administration of the compound. Thus, in some embodiments, provided are methods that include administering to a subject a therapeutically effective amount of any of the compounds of the present disclosure. In certain aspects, provided are methods of delivering a compound to a target site in a subject, the method including administering to the subject a pharmaceutical composition including any of the compounds of the present disclosure, where the administering is effective to provide a therapeutically effective amount of the compound at the target site in the subject.

The subject to be treated can be one that is in need of therapy, where the subject to be treated is one amenable to treatment using the compounds disclosed herein. Accordingly, a variety of subjects may be amenable to treatment using the compounds disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The present disclosure provides methods that include delivering a compound of the present disclosure to an individual having Alzheimer's disease, such as methods that include administering to the subject a therapeutically effective amount of a compound of the present disclosure. The methods are useful for treating a wide variety of conditions and/or symptoms associated with Alzheimer's disease. In the context of Alzheimer's disease, the term “treating” includes one or more (e.g., each) of: reducing the severity of one or more symptoms, inhibiting the progression, reducing the duration of one or more symptoms, and ameliorating one or more symptoms associated with Alzheimer's disease. In certain embodiments, methods of the present disclosure include administering a compound of the present disclosure to a subject, where the administering is effective for treating a disease mediated through activity of the amylin receptor. In some instances, compounds of the present disclosure are effective for inhibiting the activity of the amylin receptor.

The compounds described herein can be isolated by procedures known to those skilled in the art. The compounds described herein may be obtained, for instance, by a resolution technique or by chromatography techniques (e.g., silica gel chromatography, chiral chromatography, etc.). As used herein, the term “isolated” refers to compounds that are non-naturally occurring and can be obtained or purified from synthetic reaction mixtures. Isolated compounds may find use in the pharmaceutical compositions and methods of treatment described herein.

The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Thus, the disclosed compounds may be enriched in one or more of these isotopes relative to the natural abundance of such isotope. By way of example, deuterium (2H; D) has a natural abundance of about 0.015%. Accordingly, for approximately every 6,500 hydrogen atoms occurring in nature, there is one deuterium atom. Specifically contemplated herein are compounds enriched in deuterium at one or more positions. Thus, deuterium containing compounds of the disclosure have deuterium at one or more positions (as the case may be) in an abundance of greater than 0.015%. In some embodiments, one or more (e.g., 1, 2, 3, 4, 5, 6, 7 or more) hydrogen atoms of a substituent group (e.g., an R-group) of any one of the subject compounds described herein are substituted with a deuterium.

Pharmaceutical Compositions

In certain embodiments, the disclosed compounds are useful for the treatment of a disease or disorder, such as Alzheimer's disease. Accordingly, pharmaceutical compositions comprising at least one disclosed compound are also described herein. For example, the present disclosure provides pharmaceutical compositions that include a therapeutically effective amount of a compound of the present disclosure (or a pharmaceutically acceptable salt or solvate or hydrate or stereoisomer thereof) and a pharmaceutically acceptable excipient.

A pharmaceutical composition that includes a subject compound may be administered to a patient alone, or in combination with other supplementary active agents. For example, one or more compounds according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A compound of the present disclosure may be administered to a subject using any convenient means capable of resulting in the desired reduction in disease condition or symptom. Thus, a compound can be incorporated into a variety of formulations for therapeutic administration. More particularly, a compound can be formulated into pharmaceutical compositions by combination with appropriate pharmaceutically acceptable excipients, carriers or diluents, and may be formulated into preparations in solid, semi-solid, liquid or gaseous forms, such as tablets, capsules, powders, granules, ointments, solutions, suppositories, injections, inhalants, aerosols, and the like.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of the disclosed compounds. Pharmaceutical compositions that include at least one of the compounds can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable excipient in addition to at least one active ingredient, such as a compound of the present disclosure. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. In addition to biologically neutral carriers, pharmaceutical compositions to be administered can optionally contain non-toxic auxiliary substances (e.g., excipients), such as wetting or emulsifying agents, preservatives, and pH buffering agents, and the like. The disclosed pharmaceutical compositions may be formulated as a pharmaceutically acceptable salt of a disclosed compound.

In some embodiments, the disclosed pharmaceutical compositions may be formulated to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. A BBB disrupting agent can be co-administered with the pharmaceutical compositions disclosed herein when the compositions are administered by intravenous injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to a compound disclosed herein for use in the methods disclosed herein to facilitate transport across the epithelial wall of the blood vessel. Alternatively, drug delivery behind the BBB may be by intrathecal delivery of therapeutics, e.g., administering the disclosed pharmaceutical compositions directly to the cranium, as through an Ommaya reservoir.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, excipient, carrier or vehicle. The specifications for a compound depend on the particular compound employed and the effect to be achieved, and the pharmacodynamics associated with each compound in the subject.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual compound may be administered at the same time as another therapeutic compound or sequentially, in any order thereof.

A disclosed compound can be administered alone, as the sole active pharmaceutical agent, or in combination with one or more additional compounds of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

Methods of Administration

The subject compounds find use for treating a disease or disorder in a subject, such as Alzheimer's disease. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, intrathecal, and transdermal. Formulations for these dosage forms are described herein.

An effective amount of a subject compound may depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (e.g., patient) being treated. For example, this may be the amount of a subject compound necessary to prevent, inhibit, reduce or relieve a disease or disorder in a subject, such as Alzheimer's disease. Ideally, a therapeutically effective amount of a compound is an amount sufficient to prevent, inhibit, reduce or relieve a disease or disorder in a subject without causing a substantial cytotoxic effect on host cells in the subject.

Therapeutically effective doses of a subject compound or pharmaceutical composition can be determined by one of skill in the art. For example, in some instances, a therapeutically effective dose of a compound or pharmaceutical composition is administered with a goal of achieving local (e.g., tissue) concentrations that are at least as high as the EC₅₀ of an applicable compound disclosed herein.

The specific dose level and frequency of dosage for any particular subject may be varied and may depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

In some embodiments, multiple doses of a compound are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/l, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds, including compounds that are not commercially available, can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

In certain embodiments, compounds of Formula (I) are synthesized using methods and conditions that are known to one of ordinary skill in the art, as depicted in Scheme 1:

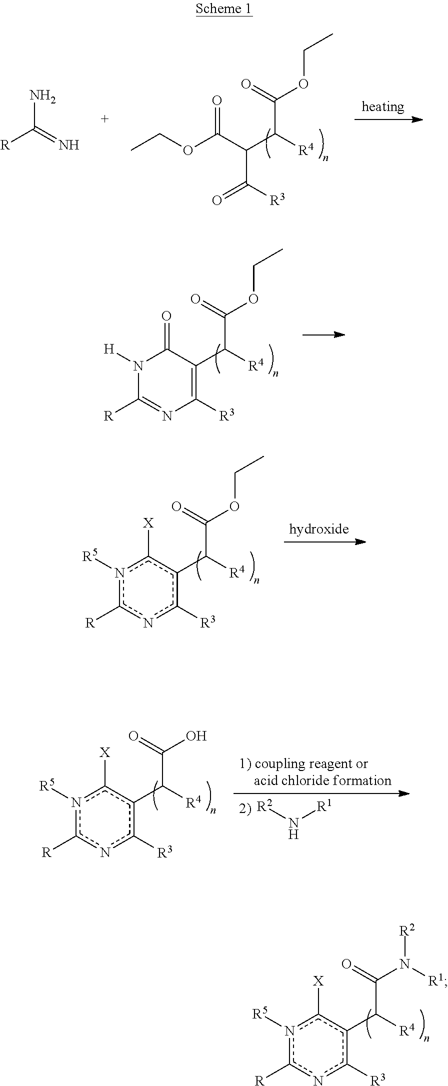

wherein R, R¹, n, R², R³, R⁴, R⁵, and X are as defined herein.
The starting materials and reagents employed in Scheme 1 may be obtained commercially or through techniques known to one of ordinary skill in the art.

In certain embodiments, compounds of Formula (II) are synthesized using methods and conditions that are known to one of ordinary skill in the art, as depicted in Scheme 2:

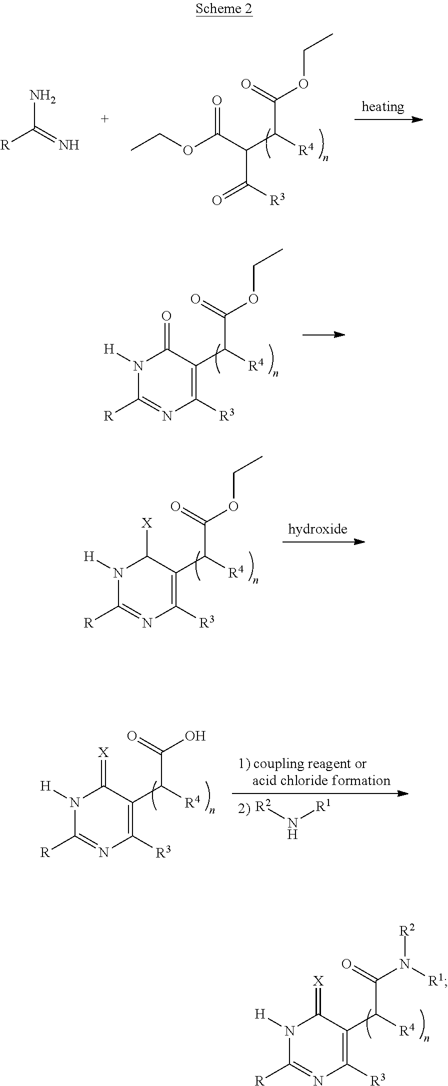

wherein R, R¹, n, R², R³, R⁴, and X are as defined herein.
The starting materials and reagents employed in Scheme 2 may be obtained commercially or through techniques known to one of ordinary skill in the art.

In certain embodiments, compounds of Formula (III) are synthesized using methods and conditions that are known to one of ordinary skill in the art, as depicted in Scheme 3:

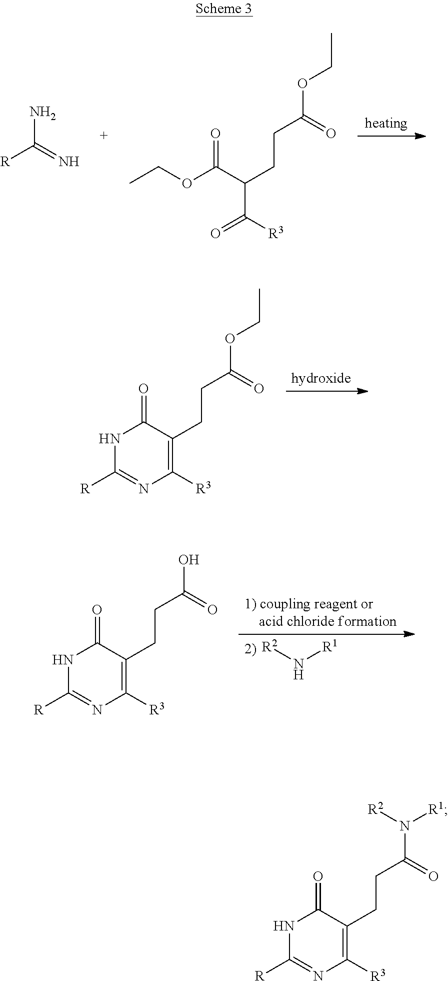

wherein R, R¹, R² and R³ are as defined herein.
The starting materials and reagents employed in Scheme 3 may be obtained commercially or through techniques known to one of ordinary skill in the art.

In certain embodiments, compounds of Formula (IV) are synthesized using methods and conditions that are known to one of ordinary skill in the art, as depicted in Scheme 4:

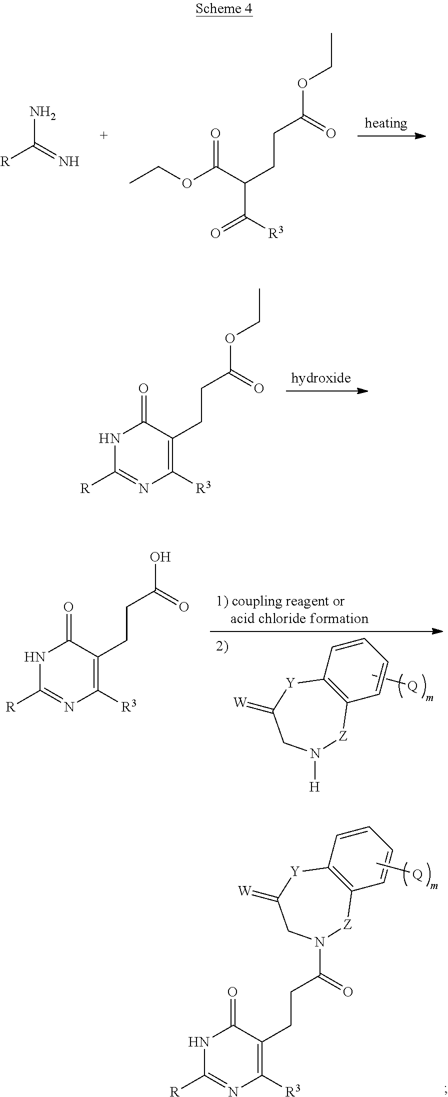

wherein W, Y, m, R, R³, Q and Z are as defined herein.
The starting materials and reagents employed in Scheme 4 may be obtained commercially or through techniques known to one of ordinary skill in the art.

Schemes 1, 2, 3 and 4 are meant to be by way of non-limiting examples only, and one of ordinary skill in the art will understand that alternate reagents, solvents or starting materials can be used to make compounds of Formula (I) and/or (II) and/or (III) and/or (IV) and/or other compounds contained herein.

### Example 1: Synthesis of Compounds

All reagents and solvents were used as purchased from commercial sources.

Moisture sensitive reactions were carried out under a nitrogen atmosphere. Reactions were monitored by TLC using pre-coated silica gel aluminum plates containing a fluorescent indicator (F-254). Detection was done with UV (254 nm). Alternatively the progress of a reaction was monitored by LC/MS. Specifically, but without limitation, the following abbreviations were used, in addition to the other ones described herein, in the examples: cat. (catalytic amount); DCM (dichloromethane); dioxane (1,4-dioxane); DIPEA (N,N-diisopropylethylamine); DMF (N,N-dimethylformamide); EtOH (ethanol); ether or Et₂O (diethyl ether); Et₃N (triethylamine); HATU (1-[bis(dimethylamino)methylene]-1H-1,2,3-triazolo[4,5-b]pyridinium 3-oxide hexafluorophosphate or N-[(dimethylamino)-1H-1,2,3-triazolo-[4,5-b]pyridin-1-ylmethylene]-N-methylmethanaminium hexafluorophosphate N-oxide); MeCN (acetonitrile); MeOH (methanol); μW (microwave); O/N (overnight); RT or rt (room or ambient temperature); THF (tetrahydrofuran). ¹H NMR spectra were recorded at RT with a Bruker Avanche III 600 MHz NMR spectrometer equipped with a Bruker's 5 mm PABBO probe. Chemical shifts are reported in ppm downfield from tetramethylsilane using residual solvent signals as internal reference. NMR data were processed utilizing ACD/Spectrus processor (v2016.1.1, ACD/Labs Inc.). Nomenclature for the naming of compounds, such as for Compound Examples and intermediate compounds, were performed using ACD/Name (Chemists' Version from ACD/Labs Inc.) to generate the IUPAC-style names. Naming of commercial or literature compounds utilized SciFinder, ACD/Names, and common or trivial names known to those skilled in the art.

Microwave assisted reactions were performed using an Anton Paar “Monowave 200” Microwave Synthesis Reactor with magnetron power 850 W. Unless stated otherwise the temperature was reached as fast as possible and controlled by built-in IR sensor (temperature uncertainty ±5° C.). Reaction was carried out either in 10 mL or 30 mL vials, with the default stirrer speed 600 rpm.

The LC/MS system used for monitoring the progress of reactions, assessing the purity (absorbance at 254 nm) and identity of the product consisted of Dionex ULTIMATE 3000 uHPLC module and Thermo Scientific LTQ XL mass-spectrometer with electrospray ionization and Ion-Trap type of detector (alternating positive-negative mode). Separation was performed with Thermo Scientific™ Accucore™ aQ C₁₈ Polar Endcapped LC column (100 mm×2.1 mm; particle size 2.6 μm, 80 Å). The column was maintained at 40° C. Commercial HPLC-grade methanol, acetonitrile and domestic ‘millipore (Milli-Q)’ water used for chromatography were modified by adding 0.1% (v/v) of formic acid. The eluent was delivered with constant flow rate of 0.4 mL/min, column was equilibrated for 5 min with the corresponding eluent prior to injection of the sample (1 μL) and one of the following separation conditions were used:

Eluent systems:
A—Gradient of MeOH-Water, 15% to 65% in 5 min, 65% to 95% in 2.5 min, followed by 4 min of isocratic MeOH—water 95%;
B—Gradient of Methanol-Water, 30 to 65% in 4.75 min, then to 95% in 2.5 min, followed by 4 min of isocratic MeOH—water 95%;
C—Gradient of MeOH-Water, 10% to 45% in 5 min, 45% to 95% in 2.5 min, followed by 4 min of isocratic MeOH—water 95%.

### General Procedure 1

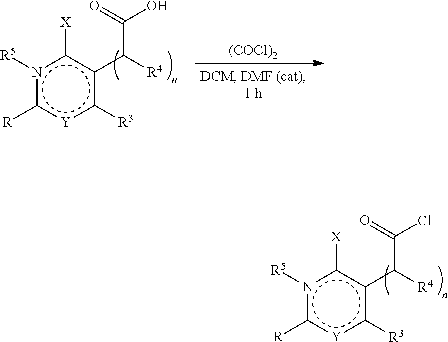

Preparation of acyl chloride from carboxylic acid. To a stirred suspension of carboxylic acid (0.56 mmol) in anhydrous DCM (10 mL) containing 2-3 drops of DMF cooled in the ice bath was added slowly oxalyl chloride (145 mg, 1.15 mmol) over 30 seconds. After 15 minutes, the ice bath was removed, the mixture was stirred at room temperature for an additional hour, then concentrated under reduced pressure. Toluene (10 mL) was added to the residue and the mixture was concentrated under reduced pressure. The product was used in the next step without further purification.

### Compound 22

### Synthesis of 4-(3-(4-methyl-6-oxo-2-phenyl-1,6-dihydropyrimidin-5-yl)propanoyl)-3,4-dihydroquinoxalin-2(1H)-one, 22

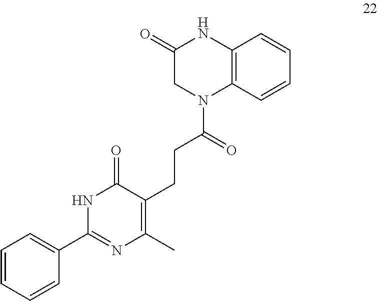 [SMILES: Cc1nc(-c2ccccc2)[nH]c(=O)c1CCC(=O)N1CC(=O)Nc2ccccc21]

Compound 22 was synthesized as in Scheme 5.

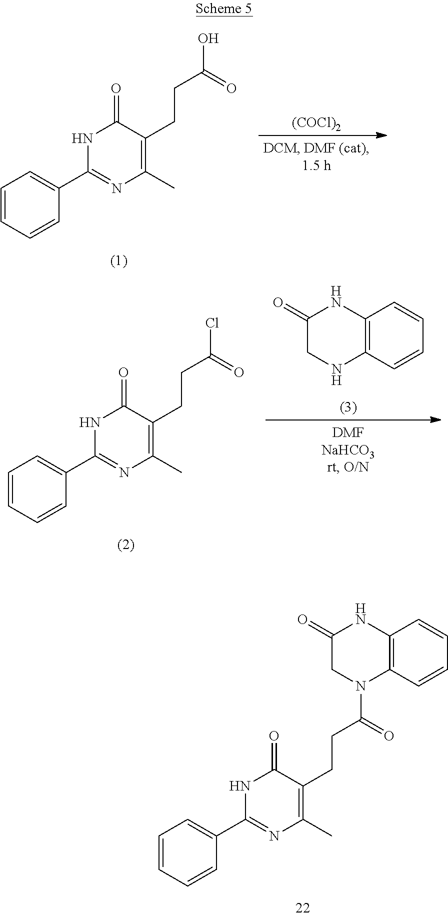

Preparation of 4-(3-(4-methyl-6-oxo-2-phenyl-1,6-dihydropyrimidin-5-yl)propanoyl)-3,4-dihydroquinoxalin-2(1H)-one, 22. To the solution of 3-(4-methyl-6-oxo-2-phenyl-1,6-dihydropyrimidin-5-yl)propanoyl chloride (2), prepared from 3-(4-methyl-6-oxo-2-phenyl-1,6-dihydropyrimidin-5-yl)propanoic acid (1) (50 mg, 0.19 mmol) following the General Procedure 1, in DMF (5 mL) were added 3,4-dihydroquinoxalin-2(1H)-one (3) (80 mg, 0.54 mmol) and sodium bicarbonate (90 mg, 1.07 mmol). After overnight at room temperature, the volatiles were removed under reduced pressure and the residue was partitioned between chloroform and water. The organic layer was dried over sodium sulfate, filtered and concentrated under reduced pressure. The resulting residue was dissolved in DCM and loaded on silica gel. Purification of the product by column chromatography performed with gradient of 1% to 8% methanol in chloroform provided 22 (43 mg, 58% yield over two steps) as a yellow solid.

¹H NMR (600 MHz, DMSO-d6) δ 12.57 (br s, 1H), 10.66 (s, 1H), 8.05 (br s, 2H), 7.58-7.45 (m, 4H), 7.18 (dd, J=7.4, 7.6 Hz, 1H), 7.01 (ddd, J=1.4, 7.6, 7.8, 1H), 6.99 (dd, J=1.3, 8.0 Hz, 1H), 4.35 (s, 2H), 2.76-2.67 (m, 4H), 2.28 (s, 3H).

LC/MS: Eluent system A (retention time: 6.58 min); ESI-MS 389.2 [M+H]⁺, 387.3 [M−H]⁻.

### Compound 23

### Synthesis of 4-(3-(2,4-dimethyl-6-oxo-1,6-dihydropyrimidin-5-yl)propanoyl)-3,4-dihydroquinoxalin-2(1H)-one, 23

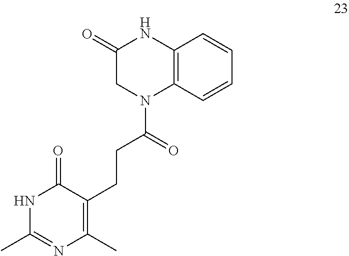 [SMILES: Cc1nc(C)c(CCC(=O)N2CC(=O)Nc3ccccc32)c(=O)[nH]1]

Compound 23 was synthesized as in Scheme 6.

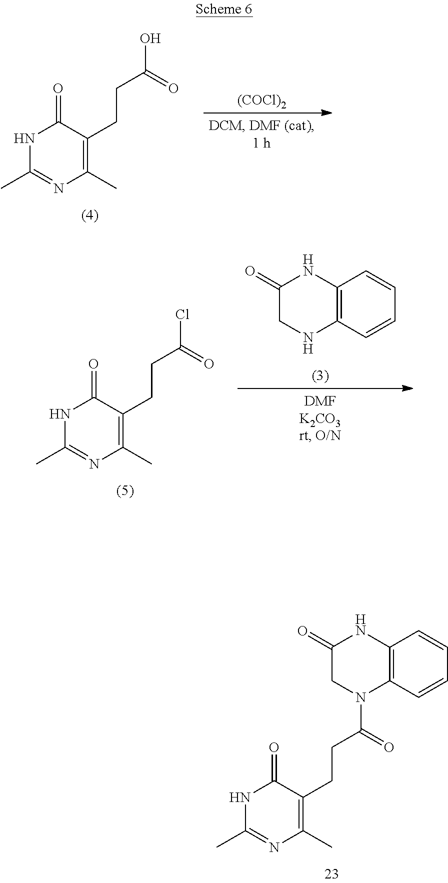

Preparation of 4-(3-(2,4-dimethyl-6-oxo-1,6-dihydropyrimidin-5-yl)propanoyl)-3,4-dihydroquinoxalin-2(1H)-one, 23. To the solution of 3-(2,4-dimethyl-6-oxo-1,6-dihydropyrimidin-5-yl)propanoyl chloride (5), prepared from 3-(2,4-dimethyl-6-oxo-1,6-dihydropyrimidin-5-yl)propanoic acid (4) (50 mg, 0.25 mmol) following the General Procedure 1, in DMF (5 mL) were added 3,4-dihydroquinoxalin-2(1H)-one (3) (42 mg, 0.28 mmol) and potassium carbonate (120 mg, 0.87 mmol). After 3 hours at room temperature, an additional portion of amine (3) (21 mg, 0.14 mmol) was added and after overnight the mixture was concentrated under reduced pressure. The residue was diluted in ethyl acetate and washed with sodium bicarbonate solution. The organic layer was dried over sodium sulfate, filtered and concentrated. The resulting residue was dissolved in DCM and loaded on a silica gel column. The column was eluted with a gradient of 1% to 7% methanol in chloroform, which generated 23 (63 mg, 80% yield over two steps) as a white amorphous powder.

¹H NMR (600 MHz, DMSO-d6) δ 12.15 (br s, 1H), 10.64 (br s, 1H), 7.44 (br s, 1H), 7.17 (br s, 1H), 7.00 (br s, 2H), 4.32 (s, 2H), 2.63 (m, 4H), 2.51 (s, 3H), 2.18 (s, 3H), 2.12 (s, 3H).

LC/MS: Eluent system A (retention time: 3.20 min); ESI-MS 327.1 [M+H]⁺, 325.3 [M−H]⁻, 371.0 [M+HCO₂⁻]⁻.

### Compound 24

### Synthesis of 5-(3-(3,4-dihydroquinoxalin-1(2H)-yl)-3-oxopropyl)-6-methyl-2-morpholinopyrimidin-4(3H)-one, 24

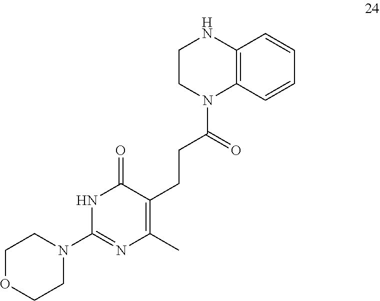 [SMILES: Cc1nc(N2CCOCC2)[nH]c(=O)c1CCC(=O)N1CCNc2ccccc21]

Compound 24 was synthesized as in Scheme 7.

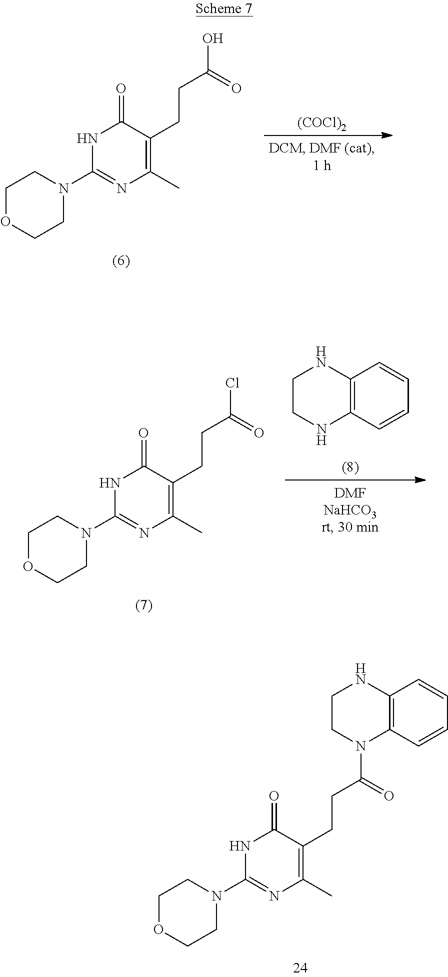

Preparation of 3-(4-methyl-2-morpholino-6-oxo-1,6-dihydropyrimidin-5-yl)propanoyl chloride (7). To a stirred suspension of 3-(4-methyl-2-morpholino-6-oxo-1,6-dihydropyrimidin-5-yl)propanoic acid (6) (150 mg, 0.56 mmol) in anhydrous DCM (10 mL) containing 2-3 drops of DMF cooled in the ice bath was added slowly oxalyl chloride (145 mg, 1.15 mmol) (over 30 seconds). After 15 minutes, the ice bath was removed, and the mixture was stirred at room temperature for additional hour, then concentrated under reduced pressure. Toluene (10 mL) was added to the residue and then evaporated under reduced pressure. The product was used in the next step without further purification.

Preparation of 5-(3-(3,4-dihydroquinoxalin-1(2H)-yl)-3-oxopropyl)-6-methyl-2-morpholinopyrimidin-4(3H)-one, 24. A solution of 3-(4-methyl-2-morpholino-6-oxo-1,6-dihydropyrimidin-5-yl)propanoyl chloride (7) (0.18 mmol) in anhydrous DMF (5 mL) was transferred into a flask equipped containing 1,2,3,4-tetrahydroquinoxaline (8) (80 mg, 0.60 mmol) and sodium bicarbonate (110 mg, 1.3 mmol). After 30 min, the solvent was evaporated under reduced pressure and the residue was partitioned between chloroform (15 mL) and 3% sodium bicarbonate solution (15 mL). The aqueous layer was washed twice with chloroform (2×15 mL), and the combined organic fraction was dried over sodium sulfate, filtered and concentrated under reduced pressure. The product was purified by column chromatography on silica gel (eluted with gradient of 1% to 7% MeOH—CHCl₃) provided 24 (43 mg, 62% yield over two steps) as a yellowish amorphous powder.

¹H NMR (600 MHz, DMSO-d6) δ 11.11 (br s, 1H), 7.05 (br s, 1H), 6.87 (br s, 1H), 6.58 (d, J=8.2 Hz, 1H), 6.44 (ddd, J=1.3, 7.2, 7.7 Hz, 1H), 6.15 (br s, 1H), 3.65 (dd, J=4.8, 5.0 Hz, 2H), 3.60 (dd, J=4.3, 4.9 Hz, 4H), 3.50 (br s, 4H), 3.26 (br s, 2H), 2.57 (br s, 4H), 2.07 (br s, 3H).

LC/MS: Eluent system A (retention time: 5.52 min); ESI-MS 384.2 [M+H]⁺, 382.4 [M−H]⁻.

### Compound 25

### Synthesis of 6-methyl-2-(morpholin-4-yl)-5-[3-oxo-3-(3-oxopiperazin-1-yl)propyl]pyrimidin-4(3H)-one, 25

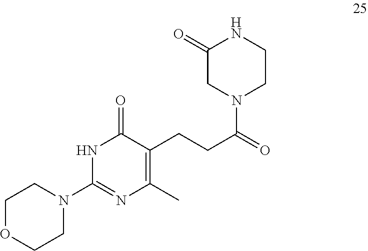 [SMILES: Cc1nc(N2CCOCC2)[nH]c(=O)c1CCC(=O)N1CCNC(=O)C1]

Compound 25 was synthesized as in Scheme 8.

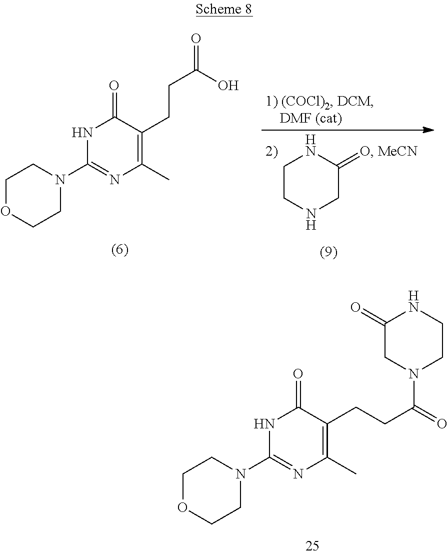

Preparation of 6-methyl-2-(morpholin-4-yl)-5-[3-oxo-3-(3-oxopiperazin-1-yl) propyl]pyrimidin-4(3H)-one, 25. In an oven-dried round-bottom flask was added with 3-[4-methyl-2-(morpholin-4-yl)-6-oxo-1,6-dihydropyrimidin-5-yl]propanoic acid (1), (50 mg, 0.19 mmol) and dichloromethane (5 mL). To this suspension at room temperature, added oxalyl chloride (0.16 mL, 1.9 mmol), followed by 2 drops of N,N-dimethylformamide. After 30 mins, the clear solutions was concentrated under reduced pressure, co-evaporated with dichloromethane (10 mL) providing an orange-yellow color solid. To this solid, was added piperazin-2-one (9) (93.6 mg, 0.94 mmol) and acetonitrile (10 mL). After 2 h, the mixture was concentrated under reduced pressure. The resulting solid was dissolved in tetrahydrofuran (20 mL), added 3-(trifluoromethyl)benzoyl chloride (150 mg, 0.72 mmol) and stirred for 30 min at room temperature. The crude was adsorbed on silica gel and purified by column purification (eluted with a gradient of 0% to 6% methanol-chloroform) afforded 25 as a white color solid (34.2 mg, 52% yield).

¹H NMR (600 MHz, DMSO-d₆) δ 11.15 (br s, 1H), 8.12-8.01 (m, 1H), 3.99 (br s, 1H), 3.91 (br s, 1H), 3.65-3.60 (m, 5H), 3.60-3.55 (m, 2H), 3.51 (br s, 4H), 3.27-3.18 (m, 1H), 3.17-3.08 (m, 1H), 2.46-2.41 (m, 2H), 2.40-2.36 (m, 1H), 2.12 (br s, 3H).

LC/MS: Eluent system A (retention time: 1.53 min); ESI-MS: 350.2 [M+H]⁺.

### Compound 26

### Synthesis of 3-(2,4-dimethyl-6-oxo-1,6-dihydropyrimidin-5-yl)-N-methyl-N-phenylpropanamide, 26

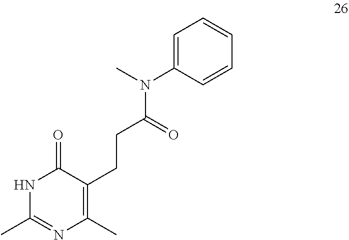 [SMILES: Cc1nc(C)c(CCC(=O)N(C)c2ccccc2)c(=O)[nH]1]

Preparation of 3-(2,4-dimethyl-6-oxo-1,6-dihydropyrimidin-5-yl)-N-methyl-N-phenylpropanamide, 26. To the mixture of 3-(2,4-dimethyl-6-oxo-1,6-dihydropyrimidin-5-yl)propanoic acid (4) (50 mg, 0.25 mmol), N-methylaniline (10) (54 mg, 0.50 mmol), and triethylamine (0.35 mL, 2.5 mmol) in anhydrous DMF (8 mL) was added HATU (114 mg, 0.30 mmol). After overnight at room temperature, an additional portion of HATU (114 mg, 0.30 mmol) was added to the mixture. After an additional 24 h, the resulting mixture was concentrated under reduced pressure, and the residue was partitioned between chloroform and sodium bicarbonate solution. The separated organic layer was dried over sodium sulfate, filtered and concentrated under reduced pressure. The product was purified by silica gel column chromatography (eluted with a gradient of 0% to 5% MeOH—CHCl₃) providing 26 as an off-white solid (21 mg, 29% yield).

¹H NMR (600 MHz, CDCl₃) δ 11.82 (br s, 1H), 7.37 (dd, J=7.7, 7.8 Hz, 2H), 7.27 (m, 1H, overlapped with residual CHCl₃ peak), 7.14 (d, J=7.7 Hz, 2H), 3.28 (s, 3H), 2.80 (t, J=7.6 Hz, 2H), 2.36-2.31 (m, 5H), 2.29 (s, 3H).

LC/MS: Eluent system A (retention time: 5.20 min); ESI-MS 286.1 [M+H]⁺, 284.2 [M−H]⁻, 320.0 [M+HCO₂⁻]⁻.

### Compound 27

### Synthesis of 5-(3-(2H-benzo[b][1,4]oxazin-4(3H)-yl)-3-oxopropyl)-6-methyl-2-morpholinopyrimidin-4(3H)-one, 27

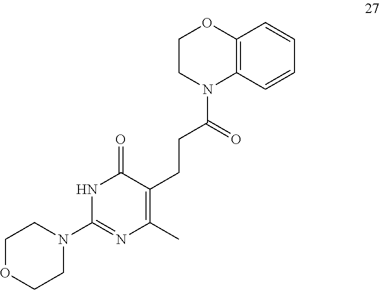 [SMILES: Cc1nc(N2CCOCC2)[nH]c(=O)c1CCC(=O)N1CCOc2ccccc21]

Preparation of 5-(3-(2H-benzo[b][1,4]oxazin-4(3H)-yl)-3-oxopropyl)-6-methyl-2-morpholinopyrimidin-4(3H)-one, 27. The solution of 3-(4-methyl-2-morpholino-6-oxo-1,6-dihydropyrimidin-5-yl)propanoyl chloride (7) (0.18 mmol), as prepared in Compound 24, in anhydrous DMF (5 mL) was transferred into a flask containing 3,4-dihydro-2H-benzo[b][1,4]oxazine (11) (130 mg, 0.96 mmol). After overnight, the reaction mixture was concentrated and partitioned between chloroform and an aqueous solution of sodium bicarbonate. The organic layer was separated, dried over sodium sulfate, filtered and concentrated under reduced pressure. The residue was dissolved in DCM and the product purified by silica gel column chromatography (eluted with a gradient of 0% to 8% MeOH—CHCl₃). The product was further purified by suspending in MeOH (5 mL), sonication for 1 min, filtering, and drying on the filter overnight, which provided 27 (36.8 mg, 53% yield over two steps) as a yellow powder.

¹H NMR (600 MHz, DMSO-d6) δ 11.14 (br s, 1H), 8.22-7.28 (m, 1H), 7.03 (br s, 1H), 6.89-6.83 (m, 2H), 4.26-4.22 (m, 2H), 3.89-3.83 (m, 2H), 3.66-3.56 (m, 4H), 3.55-3.47 (m, 4H), 2.70-2.57 (m, 4H), 2.12 (s, 3H).

LC/MS: Eluent system A (retention time: 6.39 min); ESI-MS 385.2 [M+H]⁺, 383.3 [M−H]⁻.

### Compound 28

### Synthesis of 3-(4-methyl-2-morpholino-6-oxo-1,6-dihydropyrimidin-5-yl)-N-(3-oxo-3,4-dihydro-2H-benzo[b][1,4]oxazin-8-yl)propenamide, 28

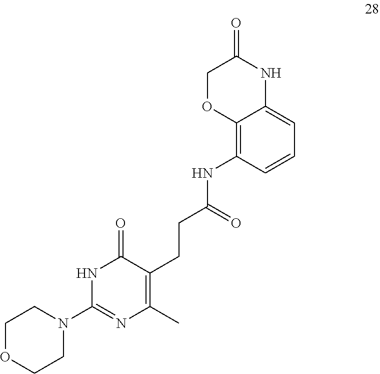 [SMILES: Cc1nc(N2CCOCC2)[nH]c(=O)c1CCC(=O)Nc1cccc2c1OCC(=O)N2]

Preparation of 3-(4-methyl-2-morpholino-6-oxo-1,6-dihydropyrimidin-5-yl)-N-(3-oxo-3,4-dihydro-2H-benzo[b][1,4]oxazin-8-yl)propenamide, 28. The solution of 3-(4-methyl-2-morpholino-6-oxo-1,6-dihydropyrimidin-5-yl)propanoyl chloride (7) (0.18 mmol), prepared as in Compound 24, in anhydrous DMF (5 mL) was transferred into a flask containing 8-amino-2H-benzo[b][1,4]oxazin-3(4H)-one (12) (80 mg, 0.48 mmol). After overnight, the reaction mixture was concentrated under reduced pressure and the resulting residue partitioned between chloroform and an aqueous solution of sodium bicarbonate. The organic layer was separated, dried over sodium sulfate, filtered and concentrated under reduced pressure. The residue was dissolved in DCM and product purified by silica gel column chromatography (eluted with a gradient of 0% to 10% MeOH—CHCl₃). The product was further purified by suspension in MeOH (5 mL), sonication for 1 min, filtering, and drying on the filter overnight, which provided 28 (4.4 mg, 6% yield over two steps).

¹H NMR (600 MHz, DMSO-d6) δ 11.16 (br s, 1H), 10.71 (s, 1H), 9.25 (s, 1H), 7.49 (d, J=7.9 Hz, 1H), 6.88 (dd, J=8.0, 8.2 Hz, 1H), 6.65 (dd, J=1.0, 8.0 Hz, 1H), 4.56 (s, 2H), 3.63-3.59 (m, 4H), 3.52 (br s, 4H), 2.64-2.58 (m, 2H), 2.49-2.44 (m, 2H), 2.14 (br s, 3H).

LC/MS: Eluent system A (retention time: 4.98 min); ESI-MS 414.2 [M+H]⁺, 412.4 [M−H]⁻.

### Compound 29

### Synthesis of N-(4-fluorophenyl)-N-methyl-3-(4-methyl-2-morpholino-6-oxo-1,6-dihydropyrimidin-5-yl)propenamide, 29

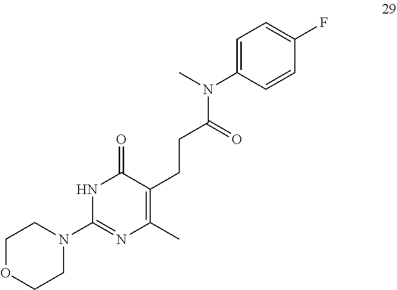 [SMILES: Cc1nc(N2CCOCC2)[nH]c(=O)c1CCC(=O)N(C)c1ccc(F)cc1]

Preparation of N-(4-fluorophenyl)-N-methyl-3-(4-methyl-2-morpholino-6-oxo-1,6-dihydropyrimidin-5-yl)propenamide, 29. The solution of 3-(4-methyl-2-morpholino-6-oxo-1,6-dihydropyrimidin-5-yl)propanoyl chloride (7) (0.18 mmol), prepared as for Compound 24, in anhydrous DMF (5 mL) was transferred into a flask containing 4-fluoro-N-methylaniline (13) (80 mg, 0.64 mmol). After overnight, the reaction mixture was concentrated under reduced pressure and the resulting residue partitioned between chloroform and an aqueous solution of sodium bicarbonate. The organic layer was separated, dried over sodium sulfate, filtered and concentrated under reduced pressure. The residue was dissolved in DCM and product purified by silica gel column chromatography (eluted with a gradient of 0% to 10% MeOH—CHCl₃). The product was further purified by suspension in MeOH (5 mL), sonication for 1 min, filtering, and drying on the filter overnight, which provided 29 (42 mg, 62% yield over two steps) as an off-white solid.

¹H NMR (600 MHz, DMSO-d6) δ 11.05 (br s, 1H), 7.34 (dd, J=4.8, 6.4 Hz, 2H), 7.24 (dd, J=8.1, 8.7 Hz, 2H), 3.61-3.57 (m, 4H), 3.48 (br s, 4H), 3.12 (s, 3H), 2.50-2.42 (m, 2H), 2.09 (dd, J=6.45, 6.73 Hz, 2H), 1.99 (br s, 3H).

LC/MS: Eluent system A (retention time: 6.25 min); ESI-MS 375.3 [M+H]⁺, 373.4 [M−H]⁻.

### Compound 30

### Synthesis of N-methyl-3-(4-methyl-2-(methylthio)-6-oxo-1,6-dihydropyrimidin-5-yl)-N-phenylpropanamide, 30

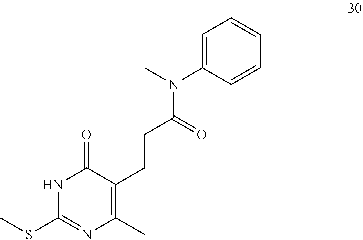 [SMILES: CSc1nc(C)c(CCC(=O)N(C)c2ccccc2)c(=O)[nH]1]

Compound 30 was synthesized as in Scheme 9.

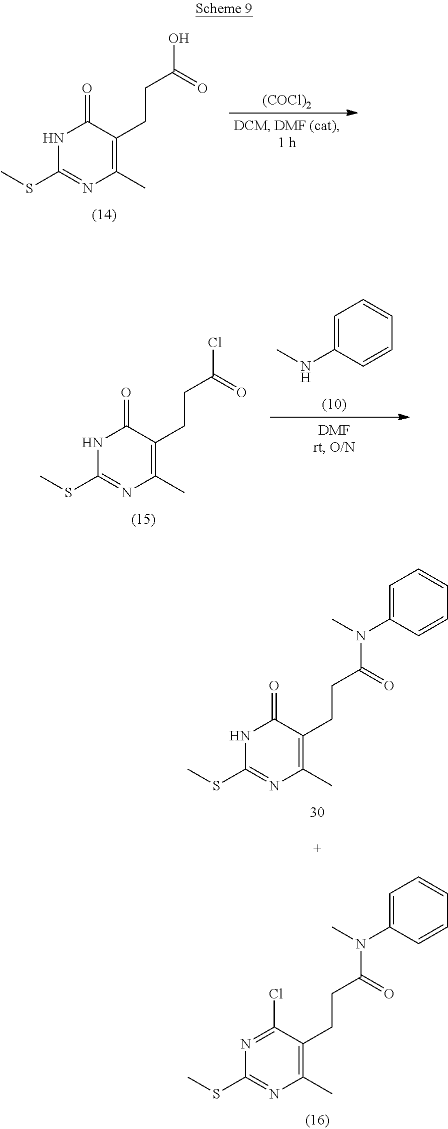

Preparation of N-methyl-3-(4-methyl-2-(methylthio)-6-oxo-1,6-dihydropyrimidin-5-yl)-N-phenylpropanamide, 30. To the solution of 3-(4-methyl-2-(methylthio)-6-oxo-1,6-dihydropyrimidin-5-yl)propanoyl chloride (15), prepared from 3-(4-methyl-2-(methylthio)-6-oxo-1,6-dihydropyrimidin-5-yl)propanoic acid (14) (80 mg, 0.35 mmol) following the General Procedure 1, in DMF (5 mL) was added N-methylaniline (10) (300 mg, 2.8 mmol). After overnight, the mixture was concentrated under reduced pressure and the resulting residue partitioned between chloroform and water. The organic layer was separated, dried over sodium sulfate, filtered and concentrated under reduced pressure. The residue was dissolved in DCM and loaded on silica gel. Purification by column chromatography with a gradient of methanol (0% to 10%) in chloroform provided 30 (6.6 mg, 6% yield over two steps), along with 3-(4-chloro-6-methyl-2-(methylthio)pyrimidin-5-yl)-N-methyl-N-phenylpropanamide (16) (38 mg, 33% yield over two steps).

For 30: ¹H NMR (600 MHz, DMSO-d6) δ 12.27 (br s, 1H), 7.52-7.14 (m, 5H), 3.15 (br s, 3H), 2.50-1.99 (m, 5H), 2.23-1.97 (m, 5H).

LC/MS: Eluent system B (retention time: 5.97 min); ESI-MS 318.2 [M+H]⁺, 316.2 [M−H]⁻.

### Compound 31

### Synthesis of 3-[2-(dimethylamino)-4-methyl-6-oxo-1,6-dihydropyrimidin-5-yl]-N-methyl-N-phenylpropanamide, 31

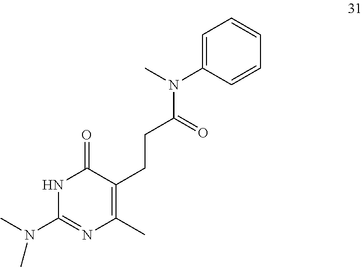 [SMILES: Cc1nc(N(C)C)[nH]c(=O)c1CCC(=O)N(C)c1ccccc1]

Compound 31 was synthesized as in Scheme 10.

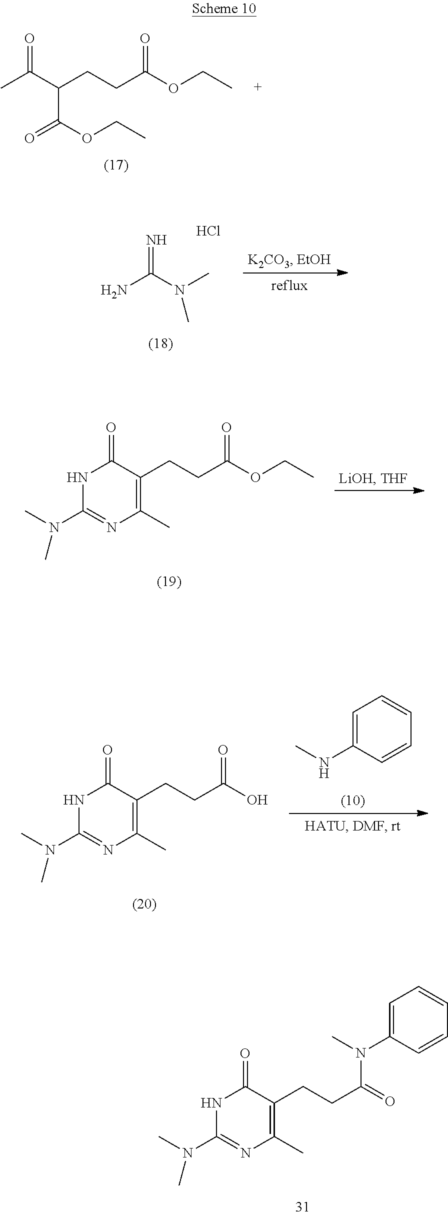

Preparation of ethyl 3-[2-(dimethylamino)-4-methyl-6-oxo-1,6-dihydropyrimidin-5-yl]propanoate, (19). To a mixture of diethyl 2-acetylglutarate (17) (870 μL, 4.05 mmol) and 1,1-dimethylguanidine hydrochloride (18) (500 mg, 4.05 mmol) in absolute ethanol (10 mL) was added K₂CO₃ (560 mg, 4.05 mmol). The resulting mixture was heated to reflux and the consumption of starting material was monitored by LC/MS. After overnight, the reaction mixture was cooled to room temperature. The mixture was filtered through a pad of Celite®. Silica gel (2 g) was added to the filtrate. The volatile components were removed under reduced pressure and the dried silica gel was loaded on column. Column chromatography was performed with a gradient of 0% to 3% MeOH in CHCl₃, which provided (19) (350 mg, 34% yield) as a white solid.

¹H NMR (600 MHz, CDCl₃) δ 10.01 (br s, 1H), 4.14 (q, J=7.2 Hz, 2H), 3.14 (s, 6H), 2.79-2.71 (m, 2H), 2.56-2.46 (m, 2H), 2.26 (s, 3H), 1.27 (t, J=7.2 Hz, 3H).

LC/MS: Eluent system A (retention time: 2.22 min); ESI-MS: 254.2 [M+H]⁺.

Preparation of 3-[2-(dimethylamino)-4-methyl-6-oxo-1,6-dihydropyrimidin-5-yl]propanoic acid, (20). To a mixture of ethyl 3-[2-(dimethylamino)-4-methyl-6-oxo-1,6-dihydropyrimidin-5-yl]propanoate (19) (150 mg, 0.59 mmol) in THF (1.5 mL) at room temperature was added 1.0 M LiOH aqueous solution (1.5 mL). The consumption of starting material was monitored by LC/MS. After 1 h, the reaction was neutralized with the 1.0 N HCl solution (1.5 mL). The pH was adjusted to 4-5 with 1 N HCl solution and then the volatile components were removed under reduced pressure. The 3-[2-(dimethylamino)-4-methyl-6-oxo-1,6-dihydropyrimidin-5-yl]propanoic acid (20) obtained was used without purification.

LC/MS: Eluent system C (retention time: 0.91 min); ESI-MS: 226.1 [M+H]⁺ and 224.1 [M−H]⁻.

Preparation of 3-[2-(dimethylamino)-4-methyl-6-oxo-1,6-dihydropyrimidin-5-yl]-N-methyl-N-phenylpropanamide, 31. To a solution of the 3-[2-(dimethylamino)-4-methyl-6-oxo-1,6-dihydropyrimidin-5-yl]propanoic acid (20) (0.59 mmol) in DMF (3 mL) at room temperature was added N-methylaniline (10) (320 μL, 2.95 mmol) and HATU (328 mg, 0.890 mmol). After overnight, the mixture was concentrated under reduced pressure, and the resulting residue was dissolved in DCM (20 mL). The resulting organic solution was washed with water (3×10 mL) and brine (10 mL), dried over sodium sulfate, filtered and concentrated under reduced pressure. The product was purified by column chromatography on silica gel with a gradient of 0% to 10% MeOH in CHCl₃ generated 31 (38.2 mg, 21% yield) as a light yellow solid.

¹H NMR (600 MHz, DMSO-d6) δ 10.74 (br s, 1H), 7.43-7.40 (m, 2H), 7.35-7.31 (m, 1H), 7.28 (dd, J=1.1, 8.3 Hz, 2H), 3.15 (br s, 3H), 2.95 (s, 6H), 2.47-2.43 (m, 2H), 2.11-2.07 (m, 2H), 1.94 (br s, 3H).

LC/MS: Eluent system A (retention time: 4.74 min); ESI-MS: 315.3 [M+H]⁺.

### Compound 32

### Synthesis of 3-(4-(dimethylamino)-6-methyl-2-(methylthio)pyrimidin-5-yl)-N-methyl-N-phenylpropanamide, 32

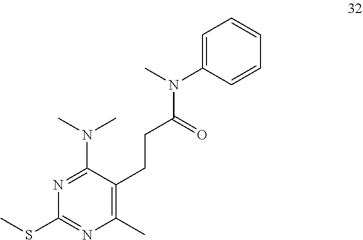 [SMILES: CSc1nc(C)c(CCC(=O)N(C)c2ccccc2)c(N(C)C)n1]

Preparation of 3-(4-(dimethylamino)-6-methyl-2-(methylthio)pyrimidin-5-yl)-N-methyl-N-phenylpropanamide, 32. In a 10 mL microwave vial equipped with a magnetic stirrer the solution of 3-(4-chloro-6-methyl-2-(methylthio)pyrimidin-5-yl)-N-methyl-N-phenylpropanamide (16) (38 mg, 0.11 mmol) in 1,4-dioxane (5 mL) was added a 2 M solution of dimethylamine in THF (3 mL). The resulting mixture was heated in a microwave reactor at 95° C. for 3 h (heated to the set temperature in 1 min). The resulting mixture was concentrated under reduced pressure, and the residue dissolved in DCM and product purified by silica gel column chromatography (eluted with gradient of 10% to 50% ethyl acetate in hexanes) provided 32 as a colorless oil (1.6 mg, 4% yield).

¹H NMR (600 MHz, DMSO-d6) δ 7.49-7.40 (m, 2H), 7.39-7.33 (m, 1H), 7.27 (d, J=7.6 Hz, 2H), 3.15 (s, 3H), 2.83 (s, 6H), 2.80-2.74 (m, 2H), 2.39 (s, 3H), 2.21-2.13 (m, 2H), 2.12 (s, 3H).

LC/MS: Eluent system B (retention time: 3.89 min); ESI-MS 345.3 [M+H]⁺.

### Compound 33

### Synthesis of N-{4-methyl-6-oxo-5-[3-oxo-3-(3-oxo-3,4-dihydroquinoxalin-1(2H)-yl)propyl]-1,6-dihydropyrimidin-2-yl}acetamide, 33

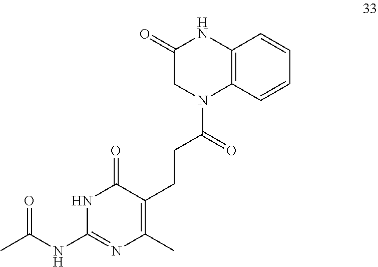 [SMILES: CC(=O)Nc1nc(C)c(CCC(=O)N2CC(=O)Nc3ccccc32)c(=O)[nH]1]

Compound 33 was synthesized as in Scheme 11.

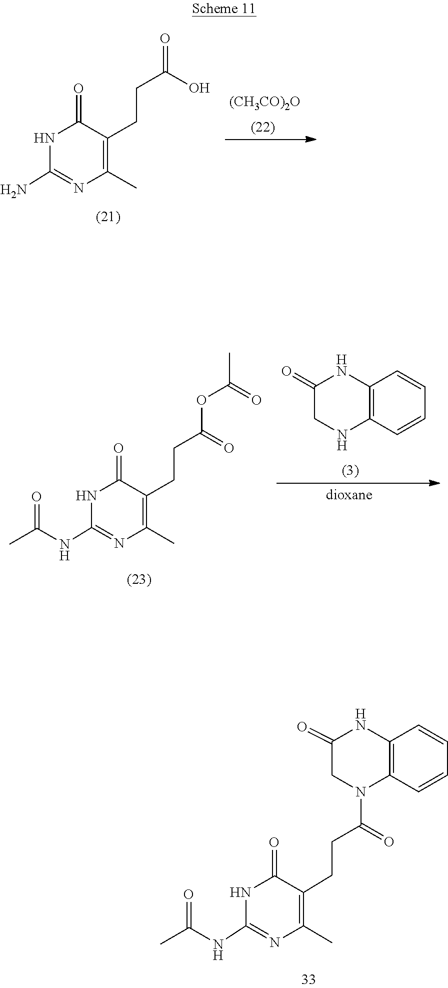

Preparation of 3-(2-acetamido-4-methyl-6-oxo-1,6-dihydropyrimidin-5-yl)propanoic acetic anhydride (23). A mixture of 3-(2-amino-4-methyl-6-oxo-1,6-dihydropyrimidin-5-yl)propanoic acid (21) (100.0 mg, 0.51 mmol) and acetic anhydride (22) (1 mL) was heated overnight at 70° C., cooled to room temperature, then concentrated under reduced pressure. The resulting gum was suspended in ether (5 mL) and filtered producing (23) (140.0 mg, 99% yield) as a white solid.

Preparation of N-{4-methyl-6-oxo-5-[3-oxo-3-(3-oxo-3,4-dihydroquinoxalin-1(2H)-yl)propyl]-1,6-dihydropyrimidin-2-yl}acetamide, 33. A mixture of 3-(2-acetamido-4-methyl-6-oxo-1,-dihydropyrimidin-5-yl)propanoic acetic anhydride (23) (70.0 mg, 0.25 mmol) and 3,4-dihydroquinoxalin-2(1H)-one (3) (111.0 mg, 0.75 mmol) in dioxane (2.5 mL) was heated to reflux. After overnight, the mixture was cooled to room temperature then concentrated under reduced pressure and the resulting residue was dissolved in DCM (2 mL). The product was purified by column chromatography on silica gel (eluted with a gradient of 0% to 5% methanol-chloroform) generated 33 (11.0 mg, 12% yield) as an off white solid.

¹H NMR (600 MHz, DMSO-d₆) δ 11.72 (br s, 1H), 11.49 (br s, 1H), 10.64 (s, 1H), 7.44 (br s, 1H), 7.18 (br t, J=7.2 Hz, 1H), 7.05-6.97 (m, 2H), 4.33 (s, 2H), 2.70-2.64 (m, 2H), 2.63-2.58 (m, 2H), 2.14 (br s, 3H), 2.12 (s, 3H).

LC/MS: Eluent system A (retention time: 4.93 min); ESI-MS: 370.2 [M+H]⁺.

### Compound 34

### Synthesis of 3-(2-amino-4-methyl-6-oxo-1,6-dihydropyrimidin-5-yl)-N-methyl-N-phenylpropanamide, 34

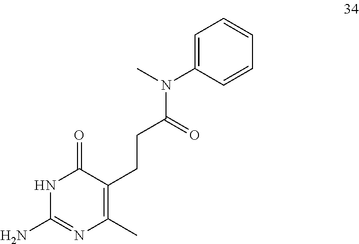 [SMILES: Cc1nc(N)[nH]c(=O)c1CCC(=O)N(C)c1ccccc1]

Preparation of 3-(2-amino-4-methyl-6-oxo-1,6-dihydropyrimidin-5-yl)-N-methyl-N-phenylpropanamide, 34. To a solution of 3-(2-amino-4-methyl-6-oxo-1,6-dihydropyrimidin-5-yl)propanoic acid (21) (70.0 mg, 0.355 mmol) in DMF (3 mL) at room temperature was added HATU (148.5 mg, 0.40 mmol) and after 30 min N-methylaniline (10) (190.2 mg, 1.8 mmol) and DIPEA (229.5 mg, 1.8 mmol) were added successively. After overnight, the mixture was concentrated under reduced pressure and dissolved in DCM (2 mL). The product was purified by column chromatography on silica gel (eluted with a gradient of 0% to 5% methanol-chloroform) provided 34 (8.8 mg, 8.6% yield) as an off white solid.

¹H NMR (600 MHz, DMSO-d₆) δ 10.55 (br s, 1H), 7.49-7.38 (m, 2H), 7.37-7.30 (m, 1H), 7.28 (br d, J=7.2 Hz, 2H), 6.19 (br s, 2H), 3.15 (s, 3H), 2.42 (br s, 2H), 2.07 (br s, 2H) 1.89 (s, 3H).

LC/MS: Eluent system B (retention time: 1.32 min); ESI-MS: 287.3 [M+H]⁺.

### Compound 35

### Synthesis of 4-[3-(4-methyl-6-oxo-1,6-dihydropyrimidin-5-yl)propanoyl]-3,4-dihydroquinoxalin-2(1H)-one, 35

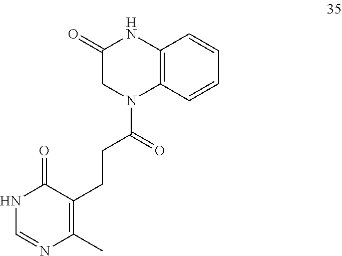 [SMILES: Cc1nc[nH]c(=O)c1CCC(=O)N1CC(=O)Nc2ccccc21]

Compound 35 was synthesized as in Scheme 12.

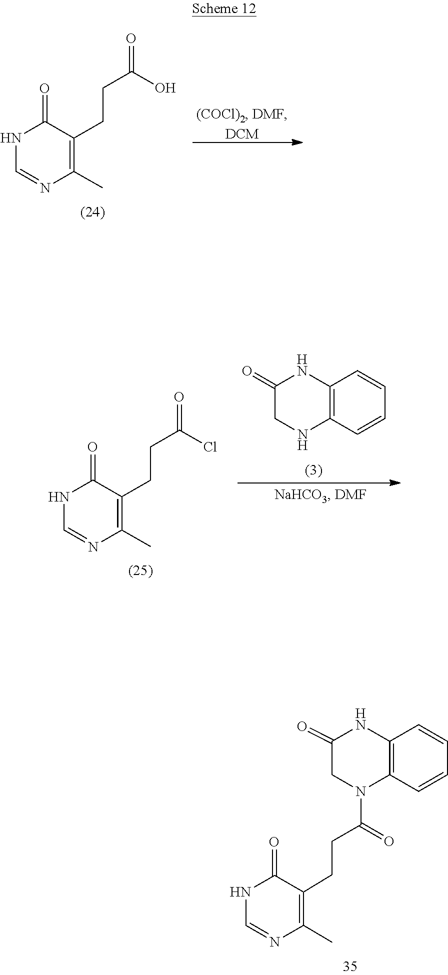

Preparation of 3-(4-methyl-6-oxo-1,6-dihydropyrimidin-5-yl)propanoyl chloride (25). To a suspension of 3-(4-methyl-6-oxo-1,6-dihydropyrimidin-5-yl)propanoic acid (24) (104.0 mg, 0.57 mmol) in DCM (5 mL) cooled in an ice bath was added oxalyl chloride (108.5 mg, 0.86 mmol) followed by DMF (0.05 mL), then after 15 min it was allowed to warm to room temperature. After 3 h, the mixture was concentrated, dried under reduced pressure to obtain (25) (152.0 mg) as a foam that was used in the next step without further purification.

Preparation of 4-[3-(4-methyl-6-oxo-1,6-dihydropyrimidin-5-yl)propanoyl]-3,4-dihydroquinoxalin-2(1H)-one, 35. To a solution of 3-(4-methyl-6-oxo-1,6-dihydropyrimidin-5-yl)propanoyl chloride (25) (125 mg, 0.30 mmol) in DMF (1.5 mL) was added 3,4-dihydroquinoxalin-2(1H)-one (3) (88 mg, 0.60 mmol) followed by NaHCO₃ (126 mg, 1.5 mmol). After overnight, the mixture was concentrated under reduced pressure, dissolved in CHCl₃ (25 mL) and filtered. The filtrate was concentrated and product purification was accomplished by column chromatography on silica gel (eluted with a gradient of 0%-5% methanol in chloroform) generated 35 (6.9 mg, 7% yield) as an off-white solid.

¹H NMR (600 MHz, DMSO-d₆) δ 12.24 (br s, 1H), 10.65 (s, 1H), 7.99-7.86 (m, 1H), 7.45 (br s, 1H), 7.22-7.13 (m, 1H), 7.04-6.96 (m, 2H), 4.33 (br s, 2H), 2.72-2.64 (m, 2H), 2.63-2.58 (m, 2H), 2.18 (br s, 3H).

LC/MS: Eluent system A (retention time: 3.82 min); ESI-MS: 313.1 [M+H]⁺.

### Compound 36

### Synthesis of N-methyl-3-(4-methyl-6-oxo-1,6-dihydropyrimidin-5-yl)-N-phenylpropanamide, 36

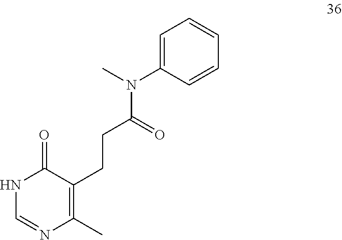 [SMILES: Cc1nc[nH]c(=O)c1CCC(=O)N(C)c1ccccc1]

Preparation of N-methyl-3-(4-methyl-6-oxo-1,6-dihydropyrimidin-5-yl)-N-phenylpropanamide, 36. To a solution of 3-(4-methyl-6-oxo-1,6-dihydropyrimidin-5-yl)propanoyl chloride (25) (125.0 mg, 0.3 mmol) in DMF (1.5 mL) was added N-methylaniline (10) (64.3 mg, 0.6 mmol) followed by NaHCO₃ (126.0 mg, 1.5 mmol). After overnight, the mixture was concentrated under reduced pressure, dissolved in CHCl₃ (25 mL) and filtered. The filtrate was concentrated and product purification was accomplished by column chromatography on silica gel (eluted with a gradient of 0% to 5% methanol—chloroform) generating the 36 (25 mg, 9% yield) as an off-white solid.

¹H NMR (600 MHz, DMSO-d₆) δ 12.20 (br s, 1H), 7.90 (br s, 1H), 7.45-7.39 (m, 2H), 7.37-7.30 (m, 1H), 7.28 (d, J=7.2 Hz, 2H), 3.15 (s, 2H), 2.55 (br s, 2H), 2.15 (br s, 3H), 2.10 (br s, 3H).

LC/MS: Eluent system A (retention time: 5.40 min); ESI-MS: 272.1 [M+H]⁺.

### Compound 37

### Synthesis of N-methyl-3-[4-methyl-6-oxo-2-(pyrrolidin-1-yl)-1,6-dihydropyrimidin-5-yl]-N-phenylpropanamide, 37

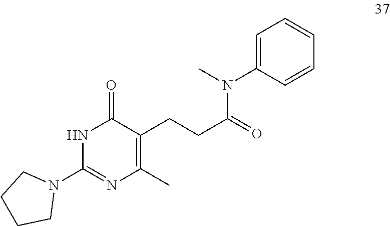 [SMILES: Cc1nc(N2CCCC2)[nH]c(=O)c1CCC(=O)N(C)c1ccccc1]

Compound 37 was synthesized as in Scheme 13.

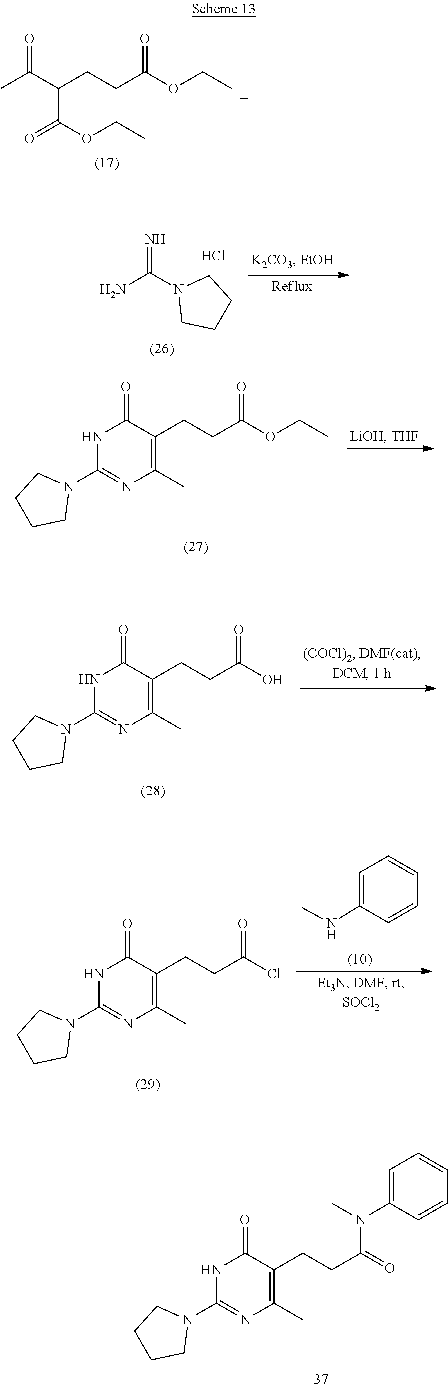

Preparation of ethyl 3-[4-methyl-6-oxo-2-(pyrrolidin-1-yl)-1,6-dihydropyrimidin-5-yl]propanoate, (27). To a mixture of diethyl 2-acetylglutarate (17) (360 μL, 1.67 mmol) and 1-pyrrolidinecarboximidamide hydrochloride (26) (250 mg, 1.67 mmol) in absolute ethanol (5 mL) was added K₂CO₃ (231 mg, 1.67 mmol). After overnight, the reaction mixture was cooled to room temperature. The mixture was filtered through a pad of Celite®. Silica gel was added to the filtrate and the solvent was removed under reduced pressure and the resulting dried silica gel was loaded on a column. Column chromatography was performed with a gradient of 0% to 3% MeOH in CHCl₃, which provided (27) (201 mg, 43% yield) as white solid.

¹H NMR (600 MHz, CDCl₃) δ 10.18 (br s, 1H), 4.14 (q, J=7.2 Hz, 2H), 3.56-3.50 (m, 4H), 2.82-2.71 (m, 2H), 2.54-2.48 (m, 2H), 2.26 (s, 3H), 2.07-1.97 (m, 4H), 1.27 (t, J=7.2 Hz, 3H).

LC/MS: Eluent system C (retention time: 4.44 min); ESI-MS: 280.2 [M+H]⁺.

Preparation of 3-[4-methyl-6-oxo-2-(pyrrolidin-1-yl)-1,6-dihydropyrimidin-5-yl]propanoic acid, (28). To a mixture of ethyl 3-[4-methyl-6-oxo-2-(pyrrolidin-1-yl)-1,6-dihydropyrimidin-5-yl]propanoate (27) (170 mg, 0.61 mmol) in THF (2 mL) at room temperature was added 1.0 M LiOH aqueous solution (2 mL). After 1.5 h, the reaction was neutralized with the same amount of 1.0 N HCl solution (2 mL). The final pH of the resulting mixture was adjusted to 5-6 with a 1.0 N HCl solution. The solvent was removed under vacuum. The material obtained (28) was used in the next step without further purification.

LC/MS: Eluent system C (retention time: 1.24 min); ESI-MS: 252.2 [M+H]⁺ and 250.1 [M−H]⁻.

Preparation of 3-[4-methyl-6-oxo-2-(pyrrolidin-1-yl)-1,6-dihydropyrimidin-5-yl]propanoyl chloride, (29). To a suspension of 3-[4-methyl-6-oxo-2-(pyrrolidin-1-yl)-1,6-dihydropyrimidin-5-yl]propanoic acid (28) in DCM (3 mL) was added oxalyl chloride (62 μL, 0.73 mmol), followed by 2 drops of DMF. After 1 h, the solvent was removed under reduced pressure. The residue was co-evaporated with DCM two times (10 mL each) and the resulting (29) was dried and used in the next step without further purification.

Preparation of N-methyl-3-[4-methyl-6-oxo-2-(pyrrolidin-1-yl)-1,6-dihydropyrimidin-5-yl]-N-phenylpropanamide, 37. To 3-[4-methyl-6-oxo-2-(pyrrolidin-1-yl)-1,6-dihydropyrimidin-5-yl]propanoyl chloride (29) in anhydrous DMF (3 mL) was added N-methylaniline (10) (100 μL, 0.92 mmol) and Et₃N (260 μL, 1.83 mmol). The reaction mixture was stirred for 30 minutes. To the reaction mixture was added SOCl₂ (46 μL, 0.64 mmol) followed by Et₃N (260 μL, 1.83 mmol). After overnight, the mixture was concentrated under reduced pressure and the resulting residue was dissolved in DCM (20 mL). The organic solution was washed with water (10 mL) and brine (10 mL). The organic phase was dried over sodium sulfate, filtered and concentrated under reduced pressure. The product was purified by column chromatography on silica (eluted with a gradient of 0% to 10% MeOH in CHCl₃) generated 37 (58.8 mg, 28% yield) as a pale yellow solid.

¹H NMR (600 MHz, CDCl₃) δ 9.65 (m, 1H), 7.38-7.34 (m, 2H), 7.28-7.26 (m, 1H), 7.15 (br d, J=7.5 Hz, 2H), 3.45 (br t, J=6.6 Hz, 4H), 3.27 (s, 3H), 2.71 (br t, J=7.7 Hz, 2H), 2.27 (br t, J=7.7 Hz, 2H), 2.16 (s, 3H), 2.04-1.96 (m, 4H).

LC/MS: Eluent system A (retention time: 4.81 min); ESI-MS: 341.3 [M+H]⁺.

### Compound 38

### Synthesis of 4-{3-[4-methyl-6-oxo-2-(pyrrolidin-1-yl)-1,6-dihydropyrimidin-5-yl]propanoyl}-3,4-dihydroquinoxalin-2(1H)-one, 38

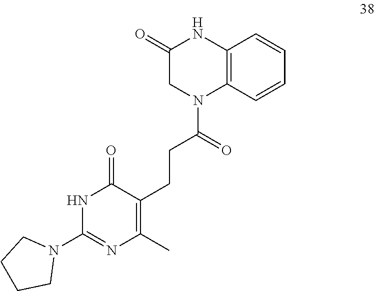 [SMILES: Cc1nc(N2CCCC2)[nH]c(=O)c1CCC(=O)N1CC(=O)Nc2ccccc21]

Preparation of 4-{3-[4-methyl-6-oxo-2-(pyrrolidin-1-yl)-1,6-dihydropyrimidin-5-yl]propanoyl}-3,4-dihydroquinoxalin-2(1H)-one, 38. To a solution of 3-[4-methyl-6-oxo-2-(pyrrolidin-1-yl)-1,6-dihydropyrimidin-5-yl]propanoic acid (28) (136 mg, 0.54 mmol), in DMF (3 mL), at room temperature was added 3,4-dihydro-1h-quinoxalin-2-one (120 mg, 0.81 mmol), Et₃N (230 μL, 1.62 mmol), and followed by SOCl₂ (43 μL, 0.59 mmol). Consumption of starting material was monitored by LC/MS and after overnight the solvent was removed under reduced pressure. The resulting residue was dissolved in DCM and the organic solution was washed with water (10 mL) and brine (10 mL). The organic phase was separated, dried over sodium sulfate, filtered and concentrated under reduced pressure. The product was purified by column chromatography on silica gel (eluted with a gradient of 0% to 10% methanol-chloroform) which generated 38 (27.6 mg, 13.4% yield) as white solid.

¹H NMR (600 MHz, CDCl₃) δ 11.37 (br s, 1H), 11.04 (br s, 1H), 7.25-7.19 (m, 1H), 7.12-7.05 (m, 1H), 7.04-6.98 (m, 1H), 6.78 (dd, J=1.1, 7.9 Hz, 1H), 4.48 (br s, 2H), 3.56 (br s, 4H), 2.90 (br s, 2H), 2.71 (br s, 2H), 2.09 (br s, 4H), 1.87 (br s, 3H).

LC/MS: Eluent system A (retention time: 3.32 min); ESI-MS: 382.3 [M+H]⁺.

### Compound 39

### Synthesis of 4-{3-[2-(dimethylamino)-4-methyl-6-oxo-1,6-dihydropyrimidin-5-yl]propanoyl}-3,4-dihydroquinoxalin-2(1H)-one, 39

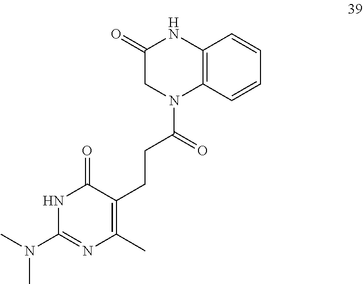 [SMILES: Cc1nc(N(C)C)[nH]c(=O)c1CCC(=O)N1CC(=O)Nc2ccccc21]

Preparation of 4-{3-[2-(dimethylamino)-4-methyl-6-oxo-1,6-dihydropyrimidin-5-yl]propanoyl}-3,4-dihydroquinoxalin-2(1H)-one, 39. To a solution of the 3-[2-(dimethylamino)-4-methyl-6-oxo-1,6-dihydropyrimidin-5-yl]propanoic acid (20) (0.47 mmol) in DMF (5 mL) at room temperature was added 3,4-dihydro-1h-quinoxalin-2-one (3) (105 mg, 0.71 mmol), Et₃N (200 μL, 1.41 mmol), followed by SOCl₂ (39 μL, 0.52 mmol). The consumption of starting material was monitored by LC/MS and after overnight the solvent was removed under reduced pressure. The resulting residue was dissolved in DCM (10 mL) and the organic solution was washed with water (10 mL) and brine (10 mL). The organic phase was separated, dried over Na₂SO₄, filtered and concentrated under reduced pressure. The product was purified by column chromatography on silica gel (eluted with a gradient of 0% to 10% methanol-chloroform), which generated 39 (15 mg, 9.0% yield) as a white solid.

¹H NMR (600 MHz, CDCl₃) δ 11.51-11.36 (m, 1H), 11.14-11.03 (m, 1H), 7.26-7.21 (m, 1H), 7.14-7.08 (m, 1H), 7.06-7.00 (m, 1H), 6.84 (dd, J=1.5, 7.9 Hz, 1H), 4.60-4.41 (m, 2H), 3.19 (s, 6H), 2.97-2.86 (m, 2H), 2.77-2.66 (m, 2H), 1.89 (br s, 3H).

LC/MS: Eluent system A (retention time: 2.93 min); ESI-MS: 356.3 [M+H]⁺.

### Compound 40

### Synthesis of 4-[4-(2,4-dimethyl-6-oxo-1,6-dihydropyrimidin-5-yl)butanoyl]-3,4-dihydroquinoxalin-2(1H)-one, 40

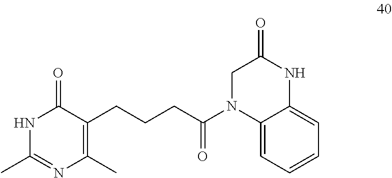 [SMILES: Cc1nc(C)c(CCCC(=O)N2CC(=O)Nc3ccccc32)c(=O)[nH]1]

Compound 40 was synthesized as in Scheme 14.

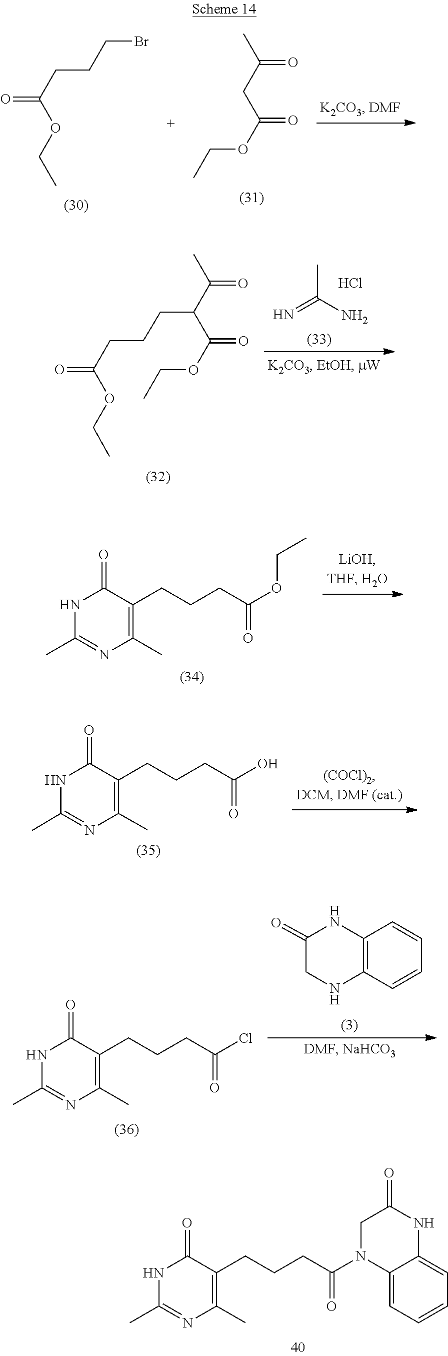

Preparation of diethyl 2-acetylhexanedioate, (32). To a solution of ethyl 4-bromobutanoate (30) (9.75 g, 50.0 mmol) and ethyl 3-oxobutanoate (31) (5.0 g, 38.5 mmol) in DMF (15 mL) was added potassium carbonate (8.0 g, 57.8 mmol). After overnight, the mixture was partitioned between ethyl acetate (100 mL) and water (20 mL). The organic layer was separated, the aqueous layer was extracted with ethyl acetate (3×50 mL) and the combined organic layer was washed with brine (20 mL), dried over sodium sulfate, filtered and concentrated under reduced pressure. The product was purified by column chromatography on silica gel (eluted with a gradient of 0%-25% ethyl acetate-hexane) generated (32) (4.8 g, 52% yield) as a yellow oil.

Preparation of ethyl 4-(2,4-dimethyl-6-oxo-1,6-dihydropyrimidin-5-yl)butanoate, (34). A mixture of diethyl 2-acetylhexanedioate (32) (1.0 g, 4.1 mmol), methylamidine hydrochloride (33) (0.58 g, 6.2 mmol) and potassium carbonate (1.1 g, 8.2 mmol) in ethanol (15 mL) was placed in a microwave reactor that was set to 120° C. for 3 h and then filtered, concentrated under reduced pressure. The resulting residue was dissolved in chloroform (2 mL). The product was purified by column chromatography on silica gel (eluted with a gradient of 10%-50% ethyl acetate-hexane) producing (34) (0.45 g, 46% yield) as a clear gum.

Preparation of 4-(2,4-dimethyl-6-oxo-1,6-dihydropyrimidin-5-yl)butanoic acid, (35). To a solution of ethyl 4-(2,4-dimethyl-6-oxo-1,6-dihydropyrimidin-5-yl)butanoate (34) (0.45 g, 1.9 mmol) in THF (10 mL) at room temperature was added lithium hydroxide (136.8 mg, 5.7 mmol, in 2 mL of water). After 3 h, the mixture was concentrated under reduced pressure, neutralized to pH 7 with 1 N HCl. The resulting mixture was concentrated under reduced pressure, dissolved in chloroform (25 mL) and filtered. The product was purified by column chromatography on silica gel (eluted with a gradient of 5%-20% methanol-chloroform) produced (35) (273.2 mg, 68% yield) as a clear gum.

Preparation of 4-[4-(2,4-dimethyl-6-oxo-1,6-dihydropyrimidin-5-yl)butanoyl]-3,4-dihydroquinoxalin-2(1H)-one, 40. To an ice-cooled suspension of 4-(2,4-dimethyl-6-oxo-1,6-dihydropyrimidin-5-yl)butanoic acid (35) (100.0 mg, 0.48 mmol) in DCM (10 mL) was added slowly oxalyl chloride (121.9 mg, 0.96 mmol) followed by DMF (0.05 mL). After warming to room temperature and stirring for 3 h, the mixture was concentrated under reduced pressure and dried under high vacuum. The resulting foam, (36), was mixed with 3,4-dihydroquinoxalin-2(1H)-one (3) (148.0 mg, 0.96 mmol) and NaHCO₃ (0.42 g, 5 mmol) in DMF (3 mL). After overnight, the mixture was concentrated under reduced pressure. The resulting residue was dissolved in chloroform (10 mL), solid removed by filtration and the product purified by column chromatography on silica gel (eluted with a gradient of ethyl acetate 0%-5% methanol-chloroform) produced 40 (22.0 mg, 13% yield) as a gray solid.

¹H NMR (600 MHz, DMSO-d₆) δ 12.17 (br s, 1H), 10.67 (s, 1H), 7.54-7.37 (m, 1H), 7.27-7.11 (m, 1H), 7.11-6.93 (m, 2H), 4.32 (s, 2H), 2.55 (br s, 2H), 2.32 (br s, 2H), 2.18 (s, 3H), 2.09 (br s, 3H), 1.70-1.57 (m, 2H).

LC/MS: Eluent system A (retention time: 4.66 min); ESI-MS: 341.3 [M+H]⁺.

### Compound 41

### Synthesis of 5-(3-(3,4-dihydroquinoxalin-1(2H)-yl)-3-oxopropyl)-2,6-dimethylpyrimidin-4(3H)-one, 41

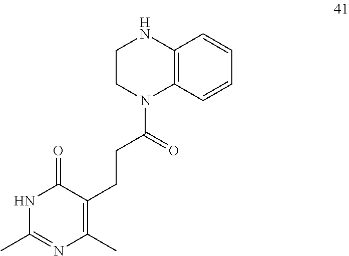 [SMILES: Cc1nc(C)c(CCC(=O)N2CCNc3ccccc32)c(=O)[nH]1]

Preparation of 5-(3-(3,4-dihydroquinoxalin-1(2H)-yl)-3-oxopropyl)-2,6-dimethylpyrimidin-4(3H)-one, 41. To the solution of 3-(2,4-dimethyl-6-oxo-1,6-dihydropyrimidin-5-yl)propanoyl chloride (5), prepared from 3-(2,4-dimethyl-6-oxo-1,6-dihydropyrimidin-5-yl)propanoic acid (4) (68 mg, 0.34 mmol) following the General Procedure 1, in DMF (5 mL) were added 1,2,3,4-tetrahydroquinoxaline (8) (100 mg, 0.74 mmol) and potassium carbonate (120 mg, 0.87 mmol). After overnight at ambient temperature, the mixture was diluted with chloroform (30 mL) and washed with 3% aqueous sodium bicarbonate solution (3×15 mL). The organic layer was dried over sodium sulfate, filtered, and concentrated under reduced pressure. The resulting residue was dissolved in DCM and loaded on silica gel column. The column was eluted with a gradient of 0% to 10% methanol in chloroform. The eluted product required additional purification, which was performed by a second silica gel column chromatography (eluted with a gradient of 9:1 to 1:1 of ethyl acetate—20% methanol in chloroform) provided 41 (10 mg, 9% yield over two steps) as an off-white solid.

¹H NMR (600 MHz, DMSO-d6) δ 12.18 (br s, 1H), 7.04 (br s, 1H), 6.87 (br s, 1H), 6.58 (d, J=7.7 Hz, 1H), 6.44 (td, J=1.3, 7.4 Hz, 1H), 6.15 (br s, 1H), 3.64 (t, J=5.0 Hz, 2H), 3.29-3.21 (m, 2H), 2.60 (br s, 4H), 2.19 (br s, 3H), 2.13 (br s, 3H).

LC/MS: Eluent system A (retention time: 4.10 min); ESI-MS 313.3 [M+H]⁺, 311.3 [M−H]⁻, 357.0 [M+HCO₂⁻]⁻.

### Compound 42

### Synthesis of 5-(3-(4-acetyl-3,4-dihydroquinoxalin-1(2H)-yl)-3-oxopropyl)-2,6-dimethylpyrimidin-4(3H)-one, 42

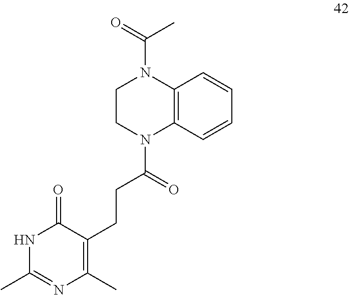 [SMILES: CC(=O)N1CCN(C(=O)CCc2c(C)nc(C)[nH]c2=O)c2ccccc21]

Preparation of 5-(3-(4-acetyl-3,4-dihydroquinoxalin-1(2H)-yl)-3-oxopropyl)-2,6-dimethylpyrimidin-4(3H)-one, 42. Acetic anhydride (22) (30 mg, 0.21 mmol) was added to the solution of 5-(3-(3,4-dihydroquinoxalin-1(2H)-yl)-3-oxopropyl)-2,6-dimethylpyrimidin-4(3H)-one 41 (30 mg, 0.09 mmol) in anhydrous pyridine (3 mL). After 15 min at room temperature, an extra portion of acetic anhydride (60 mg, 0.42 mmol) was added, and 30 min later methanol (3 mL) was added and the mixture was concentrated under reduced pressure. Toluene (5 mL) was added to the residue and solution was concentrated under reduced pressure. The product was purified by silica gel column chromatography (eluted with a gradient of 0% to 6.5% methanol in chloroform) provided 42 (8.5 mg, 25% yield) as an off-white solid.

¹H NMR (600 MHz, DMSO-d6) δ 12.19 (br s, 1H), 7.90-7.34 (m, 2H), 7.17 (br s, 2H), 3.82 (s, 4H), 2.63 (s, 4H), 2.28-2.02 (m, 9H).

LC/MS: Eluent system A (retention time: 4.57 min); ESI-MS 355.3 [M+H]⁺, 353.3 [M−H]⁻.

### Compound 43

### Synthesis of 4-[(2,4-dimethyl-6-oxo-1,6-dihydropyrimidin-5-yl)acetyl]-3,4-dihydro-quinoxalin-2(1H)-one, 43

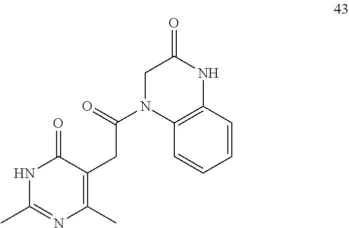 [SMILES: Cc1nc(C)c(CC(=O)N2CC(=O)Nc3ccccc32)c(=O)[nH]1]

Compound 43 was synthesized as in Scheme 15.

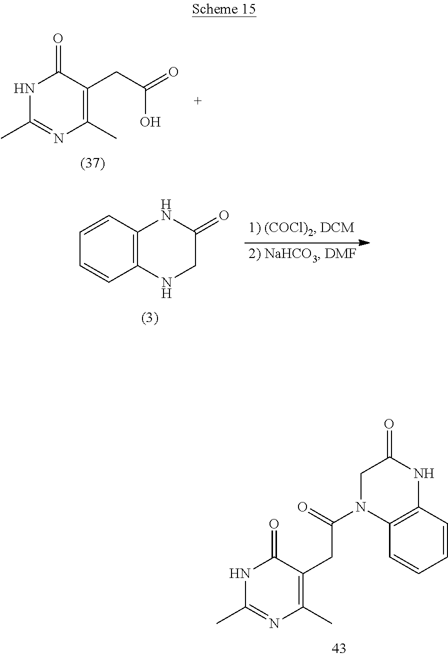 [SMILES: Cc1nc(C)c(CC(=O)O)c(=O)[nH]1 ; O=C1CNc2ccccc2N1 ; Cc1nc(C)c(CC(=O)N2CC(=O)Nc3ccccc32)c(=O)[nH]1]

Preparation of 4-[(2,4-dimethyl-6-oxo-1,6-dihydropyrimidin-5-yl)acetyl]-3,4-dihydroquinoxalin-2(1H)-one, 43. A similar procedure was followed as was described for Compound 25, but with (2,4-dimethyl-6-oxo-1,6-dihydropyrimidin-5-yl)acetic acid, (37) (50 mg, 0.27 mmol), oxalyl chloride (0.24 mL, 2.7 mmol), N,N-dimethylformamide (2 drops), dichloromethane (5 mL), 4-dihydroquinoxalin-2(1H)-one (3) (101.7 mg, 0.69 mmol), and sodium bicarbonate (115.3 mg, 1.4 mmol) in N,N-dimethylformamide (5 mL). The product was purified by silica gel column chromatography (eluted with a gradient of 0% to 8% methanol in chloroform), which produced 43 (22.6 mg, 26% yield) as an off-white color solid.

¹H NMR (600 MHz, DMSO-d₆) δ 12.26 (br s, 1H), 10.70 (s, 1H), 7.80-7.64 (m, 1H), 7.27-7.14 (m, 1H), 7.12-6.95 (m, 2H), 4.36 (br s, 2H), 3.66 (br s, 2H), 2.22 (br s, 3H), 2.13 (br s, 3H).

LC/MS: Eluent system A (retention time: 3.77 min); ESI-MS: 313.2 [M+H]⁺.

### Compound 44

### Synthesis of 4-(3-(2,4-dimethyl-6-oxo-1,6-dihydropyrimidin-5-yl)propanoyl)-1-methyl-3,4-dihydroquinoxalin-2(1H)-one, 44

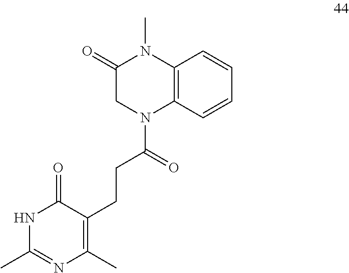 [SMILES: Cc1nc(C)c(CCC(=O)N2CC(=O)N(C)c3ccccc32)c(=O)[nH]1]

Compound 44 was synthesized as in Scheme 16.

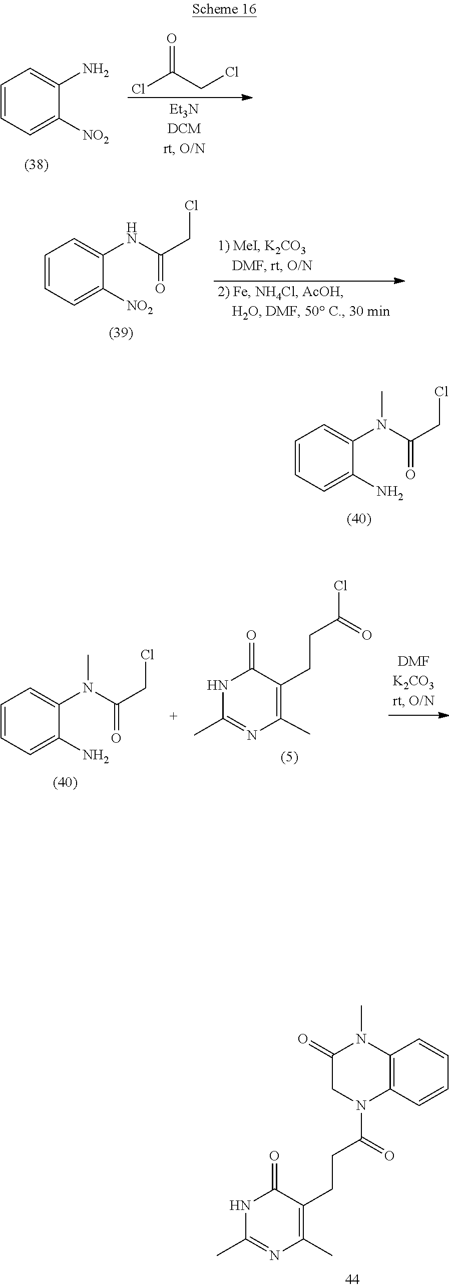

Preparation of 2-chloro-N-(2-nitrophenyl)acetamide, (39). To the solution of o-nitroaniline (38) (500 mg, 3.62 mmol) in DCM (20 mL) containing triethylamine (1.50 mL, 10.7 mmol) was added slowly chloroacetylchloride (500 mg, 4.43 mmol) over 3 minutes. After overnight at room temperature, the mixture was concentrated under reduced pressure, then the residue was dissolved in a small amount of DCM and purified by column chromatography (eluted as a gradient of 30% to 50% ethyl acetate-hexane), which provided (39) (740 mg, 95% yield) as a yellow solid.

LC/MS: Eluent system B (retention time: 4.99 min); ESI-MS 214.2 [M+H]⁺, 212.9 [M−H]⁻.

Preparation of N-(2-aminophenyl)-2-chloro-N-methylacetamide, (40). To the solution of 2-chloro-N-(2-nitrophenyl)acetamide (39) (250 mg, 1.15 mmol) in DMF (5 mL) containing an excess of methyl iodide (1.2 g, 8.5 mmol) was added potassium carbonate (350 mg, 2.5 mmol). After overnight, the mixture was concentrated under reduced pressure, and partitioned between DCM and water. The organic layer was separated, dried over sodium sulfate, filtered and concentrated under reduced pressure. The residue was dissolved in DMF (3 mL) and was added to a suspension of iron dust (650 mg, 11.6 mmol) in the aqueous solution (15 mL) of ammonium chloride (60 mg, 1.12 mmol) and acetic acid (150 mg, 2.5 mmol) that had been heated at 50° C. for 15 min. After 30 min at 50° C., saturated sodium bicarbonate solution (3 mL) was added and the resulting mixture was filtered through Celite®. The Celite® and collected solid was thoroughly washed with ethyl acetate (30 mL). The organic layer was separated from the filtrate and the water layer was extracted with ethyl acetate (3×20 mL). The organic fractions were combined, dried with sodium sulfate, filtered and concentrated under reduced pressure to provide N-(2-aminophenyl)-2-chloro-N-methylacetamide (40) (144 mg, 60% yield over two steps) that was used in the next step without further purification.

LC/MS: Eluent system A (retention time: 4.83 min); ESI-MS 163.1 [M−HCl+H]⁺.

Preparation of 4-(3-(2,4-dimethyl-6-oxo-1,6-dihydropyrimidin-5-yl)propanoyl)-1-methyl-3,4-dihydroquinoxalin-2(1H)-one, 44. A solution of 3-(2,4-dimethyl-6-oxo-1,6-dihydropyrimidin-5-yl)propanoyl chloride (5), prepared from 3-(2,4-dimethyl-6-oxo-1,6-dihydropyrimidin-5-yl)propanoic acid (4) (50 mg, 0.26 mmol) following the General Procedure 1, in DMF (5 mL) was added to the flask containing N-(2-aminophenyl)-2-chloro-N-methylacetamide (40) (70 mg, 0.3 mmol) and potassium carbonate (120 mg, 0.87 mmol). After overnight at ambient temperature, the mixture was concentrated under reduced pressure, the residue dissolved in DCM (2 mL) and the product purified by column chromatography on silica gel (eluted with a gradient of 0.2% to 8% methanol-chloroform) provided 44 (14.3 mg, 16% yield) as a white amorphous powder.

¹H NMR (600 MHz, DMSO-d6) δ 12.12 (br s, 1H), 7.48 (br s, 1H), 7.34-7.19 (m, 2H), 7.10 (dd, J=7.1, 7.2 Hz, 1H), 4.40 (s, 2H), 3.25 (s, 3H), 2.69-2.53 (m, 4H), 2.17 (br s, 3H), 2.09 (br s, 3H).

LC/MS: Eluent system C (retention time: 5.69 min); ESI-MS 341.2 [M+H]⁺, 339.3 [M−H]⁻, 384.9 [M+HCO₂⁻]⁻.

### Compound 45

### Synthesis of 4-(3-(4-methyl-2-morpholino-6-oxo-1,6-dihydropyrimidin-5-yl)propanoyl)-4,5-dihydro-1H-benzo[e][1,4]diazepin-2(3H)-one, 45

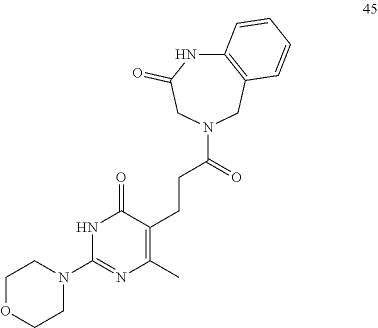 [SMILES: Cc1nc(N2CCOCC2)[nH]c(=O)c1CCC(=O)N1CC(=O)Nc2ccccc2C1]

Preparation of 4-(3-(4-methyl-2-morpholino-6-oxo-1,6-dihydropyrimidin-5-yl)propanoyl)-4,5-dihydro-1H-benzo[e][1,4]diazepin-2(3H)-one, 45. To the solution of 3-(4-methyl-2-morpholino-6-oxo-1,6-dihydropyrimidin-5-yl)propanoyl chloride (7), prepared from 3-(4-methyl-2-morpholino-6-oxo-1,6-dihydropyrimidin-5-yl)propanoic acid (6) (50 mg, 0.18 mmol) following the General Procedure 1, in DMF (5 mL) was added solid 4,5-dihydro-1H-benzo[e][1,4]diazepin-2(3H)-one (41) (40 mg, 0.25 mmol) followed by sodium bicarbonate (50 mg, 0.59 mmol). After overnight at room temperature, an additional portion of (41) (40 mg, 0.25 mmol) was added. After an additional 24 h, the mixture was concentrated under reduced pressure and the residue partitioned between chloroform and water. The organic layer was dried over sodium sulfate, concentrated under reduced pressure and the product purified by silica gel column chromatography (eluted with a gradient of 1% to 8% methanol-chloroform) provided 45 (3.1 mg, 4% yield) as a clear film.

¹H NMR (600 MHz, DMSO-d6) δ 11.16 (br s, 1H), 10.11 (s, 1H), 7.34-7.21 (m, 2H), 7.13-6.99 (m, 2H), 4.69 (s, 1H), 4.55 (s, 1H), 4.34 (s, 1H), 4.27 (s, 1H), 3.64-3.59 (m, 4H), 3.54-3.49 (m, 4H), 2.63-2.38 (m, 4H), 2.06 (s, 3H).

LC/MS: Eluent system C (retention time: 6.19 min); ESI-MS 412.4 [M+H]⁺, 410.5 [M−H]⁻.

### Compound 46

### Synthesis of 1-methyl-4-(3-(4-methyl-2-morpholino-6-oxo-1,6-dihydropyrimidin-5-yl)propanoyl)-3,4-dihydroquinoxalin-2(1H)-one, 46

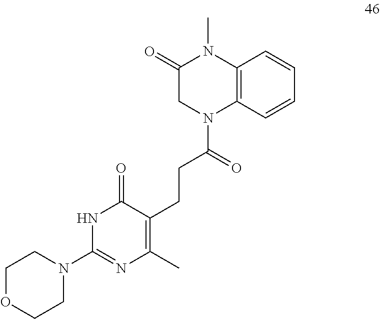 [SMILES: Cc1nc(N2CCOCC2)[nH]c(=O)c1CCC(=O)N1CC(=O)N(C)c2ccccc21]

Preparation of 1-methyl-4-(3-(4-methyl-2-morpholino-6-oxo-1,6-dihydropyrimidin-5-yl)propanoyl)-3,4-dihydroquinoxalin-2(1H)-one, 46. A solution of 3-(4-methyl-2-morpholino-6-oxo-1,6-dihydropyrimidin-5-yl)propanoyl chloride (7), prepared form 3-(4-methyl-2-morpholino-6-oxo-1,6-dihydropyrimidin-5-yl)propanoic acid (6) (50 mg, 0.19 mmol) following the General Procedure 1, in DMF (5 mL) was added to the flask containing N-(2-aminophenyl)-2-chloro-N-methylacetamide (40) (70 mg, 0.3 mmol) and potassium carbonate (120 mg, 0.87 mmol). After overnight at ambient temperature, the mixture was concentrated under reduced pressure, dissolved in DCM (2 mL) and the product purified by column chromatography on silica gel in two stages (eluted with a gradient of 0.2% to 5% methanol-chloroform followed by second column chromatography with 0% to 8% methanol-ethyl acetate) provided 46 (3.9 mg, 5% yield) as a white amorphous powder.

¹H NMR (600 MHz, DMSO-d6) δ 10.98 (br s, 1H), 7.55-7.41 (m, 1H), 7.29 (dd, J=7.7, 8.2 Hz, 1H), 7.23 (dd, J=1.0, 8.2 Hz, 1H), 7.10 (ddd, J=1.1, 7.7, 7.8 Hz, 1H), 4.41 (s, 2H), 3.62-3.58 (m, 4H), 3.50 (br s, 4H), 3.26 (s, 3H), 2.66-2.61 (m, 2H), 2.57-2.51 (m, 2H), 2.01 (br s, 3H).

LC/MS: Eluent system C (retention time: 6.88 min); ESI-MS 412.4 [M+H]⁺, 310.3 [M−H]⁻, 455.9 [M+HCO₂⁻]⁻.

### Compound 47

### Synthesis of 4-{3-[4-methoxy-6-methyl-2-(morpholin-4-yl)pyrimidin-5-yl]propanoyl}-3,4-dihydroquinoxalin-2(1H)-one, 47

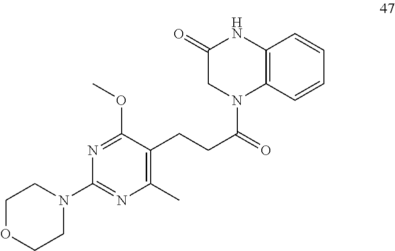 [SMILES: COc1nc(N2CCOCC2)nc(C)c1CCC(=O)N1CC(=O)Nc2ccccc21]

Compound 47 was synthesized as in Scheme 17.

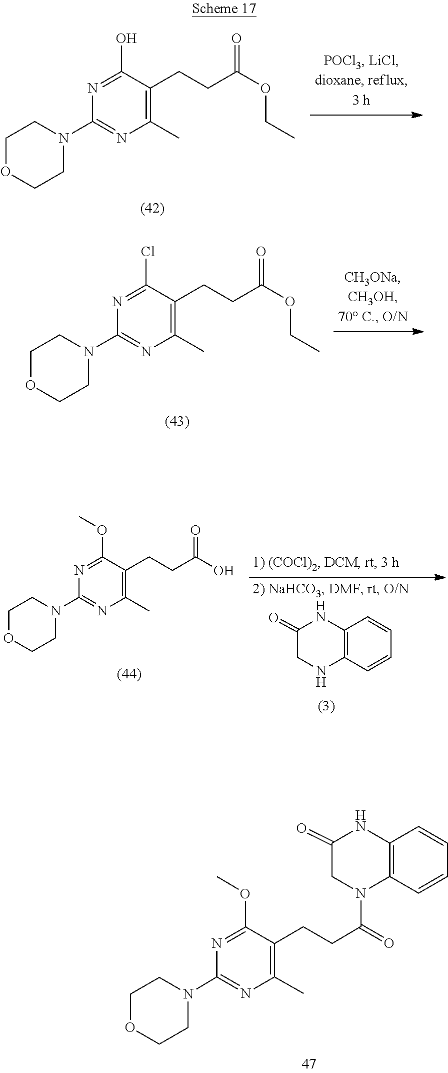 [SMILES: CCOC(=O)CCc1c(C)nc(N2CCOCC2)nc1O ; CCOC(=O)CCc1c(C)nc(N2CCOCC2)nc1Cl ; COc1nc(N2CCOCC2)nc(C)c1CCC(=O)O ; O=C1CNc2ccccc2N1 ; COc1nc(N2CCOCC2)nc(C)c1CCC(=O)N1CC(=O)Nc2ccccc21]

Preparation of ethyl 3-[4-chloro-6-methyl-2-(morpholin-4-yl)pyrimidin-5-yl]propanoate, (43). A mixture of ethyl 3-[4-hydroxy-6-methyl-2-(morpholin-4-yl)pyrimidin-5-yl]propanoate (42) (295.4 mg, 1.0 mmol), POCl₃ (230.0 mg, 1.5 mmol) and lithium chloride (42.4 mg, 1.0 mmol) in dioxane (10 mL) was heated to reflux for 3 h, after cooling to ambient temperature the mixture was concentrated under reduced pressure. The resulting residue was diluted with chloroform (50 mL) and washed with aqueous saturated NaHCO₃ solution (5 mL), dried over magnesium sulfate, filtered, and concentrated under reduced pressure. The product was purified by column chromatography on silica gel (eluted with a gradient of 0% to 50% ethyl acetate in hexane), which generated (43) (300.0 mg, 96% yield) as a white solid.

LC/MS: Eluent system B (retention time: 8.45 min); ESI-MS: 314.2 [M+H]⁺.

Preparation of 3-[4-methoxy-6-methyl-2-(morpholin-4-yl)pyrimidin-5-yl]propanoic acid, (44). A mixture of ethyl 3-[4-chloro-6-methyl-2-(morpholin-4-yl)pyrimidin-5-yl]propanoate (43) (300.0 mg, 0.96 mmol) and sodium methoxide (518.4 mg, 9.6 mmol) in methanol (25 mL) was heated to reflux. After overnight, the mixture was cooled to ambient temperature and was concentrated under reduced pressure, diluted with water (1 mL) and the pH was adjusted to 6 with 1N HCl. The resulting suspension was concentrated under reduced pressure and purification of the product was accomplished by column chromatography on silica gel (eluted with a gradient of 0% to 20% methanol in chloroform) produced (44) (110.0 mg, 40% yield) as a gray gum.

LC/MS: Eluent system C (retention time: 8.75 min); ESI-MS: 286.2 [M+H]⁺.

Preparation of 4-{3-[4-methoxy-6-methyl-2-(morpholin-4-yl)pyrimidin-5-yl]propanoyl}-3,4-dihydroquinoxalin-2(1H)-one, 47. To an ice cooled solution of 3-[4-methoxy-6-methyl-2-(morpholin-4-yl)pyrimidin-5-yl]propanoic acid (44) (110.0 mg, 0.38 mmol) in DCM (25 mL) was added slowly oxalyl chloride (73.3 mg, 0.58 mmol), followed by DMF (0.05 mL). After warming to room temperature and stirring for 2 h, the mixture was concentrated under reduced pressure. The resulting foam was mixed with 3,4-dihydroquinoxalin-2(1H)-one (3) (112.6 mg, 0.76 mmol) and NaHCO₃ (64.0 mg, 0.76 mmol) in DMF (3 mL). After overnight at room temperature, the mixture was concentrated under reduced pressure. The resulting residue was dissolved in chloroform (10 mL), solid removed by filtration and the product purified by column chromatography on silica gel (eluted with a gradient of 0% to 5% methanol in chloroform) produced 47 (18.0 mg, 11% yield) as a gray solid.

¹H NMR (600 MHz, DMSO-d₆) δ 10.62 (br s, 1H), 7.40 (br s, 1H), 7.21-7.16 (m, 1H), 7.04-6.97 (m, 2H), 4.32 (s, 2H), 3.74 (br s, 2H), 3.65-3.63 (m, 4H), 3.62 (s, 3H), 3.39-3.34 (m, 4H), 2.64 (br s, 2H), 2.17 (br s, 3H).

LC/MS: Eluent system A (retention time: 5.11 min); ESI-MS: 412.4 [M+H]⁺.

### Compound 48

### Synthesis of 4-(3-(2,4-dimethyl-6-oxo-1,6-dihydropyrimidin-5-yl)propanoyl)-4,5-dihydro-1H-benzo[e][1,4]diazepin-2(3H)-one, 48

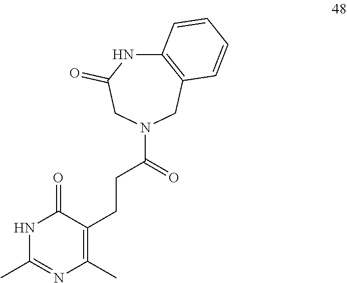 [SMILES: Cc1nc(C)c(CCC(=O)N2CC(=O)Nc3ccccc3C2)c(=O)[nH]1]

Preparation of 4-(3-(2,4-dimethyl-6-oxo-1,6-dihydropyrimidin-5-yl)propanoyl)-4,5-dihydro-1H-benzo[e][1,4]diazepin-2(3H)-one, 48. A solution of 3-(2,4-dimethyl-6-oxo-1,6-dihydropyrimidin-5-yl)propanoyl chloride (5), prepared from 3-(2,4-dimethyl-6-oxo-1,6-dihydropyrimidin-5-yl)propanoic acid (4) (50 mg, 0.26 mmol) following General Procedure 1 (modified by adding molecular sieves (4 Å, 1 g) to the reaction mixture), in DMF (5 mL) was added in portions to a solution of 4,5-dihydro-1H-benzo[e][1,4]diazepin-2(3H)-one (41) (50 mg, 0.3 mmol) in DMF (3 mL). After 2 h, to the resulting mixture was added sodium bicarbonate (90 mg, 1.07 mmol). After overnight, the resulting solution was decanted, diluted with water (1 mL), neutralized with acetic acid (250 mg, 4.2 mmol), and concentrated under reduced pressure. The product was purified by silica gel column chromatography (eluted with a gradient of 0% to 12% methanol-chloroform) provided 48 (60 mg, 70% yield) as a white powder.

¹H NMR (600 MHz, DMSO-d6) δ 12.23 (br s, 1H), 10.10 (s, 1H), 7.36-7.21 (m, 2H), 7.14-6.99 (m, 2H), 4.68 (s, 1H), 4.54 (s, 1H), 4.34 (s, 1H), 4.26 (s, 1H), 2.63-2.38 (m, 4H), 2.20 (s, 3H), 2.11 (br s, 3H).

LC/MS: Eluent system A (retention time: 3.35 min); ESI-MS 341.3 [M+H]⁺, 339.2 [M−H]⁻.

### Compound 49

### Synthesis of 4-(3-(2,4-dimethyl-6-oxo-1,6-dihydropyrimidin-5-yl)propanoyl)-7-methyl-3,4-dihydroquinoxalin-2(1H)-one, 49

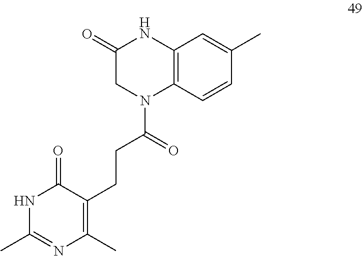

Compound 49 was synthesized as in Scheme 18.

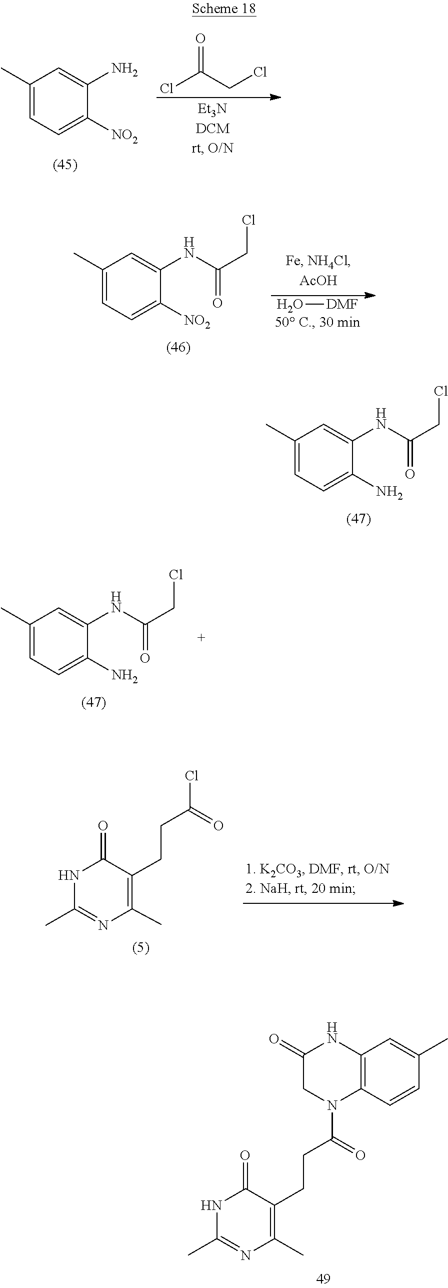

Preparation of 2-chloro-N-(5-methyl-2-nitrophenyl)acetamide (46). To a solution of 5-methyl-2-nitroaniline (45) (500 mg, 3.30 mmol) in anhydrous DMF (10 mL) containing potassium carbonate (690 mg, 5.0 mmol) was added chloroacetylchloride (920 mg, 8.1 mmol) slowly over 1 minute. After overnight, the mixture was concentrated under reduced pressure and the residue partitioned between chloroform and 1 M HCl. The organic layer was separated, dried over anhydrous sodium sulfate, filtered and concentrated under reduced pressure. The product was purified by recrystallization from methanol providing (46) (680 mg, 90% yield) as a yellow powder.

LC/MS: Eluent system B (retention time: 6.82 min); ESI-MS 229.1 [M+H]⁺, 227.0 [M−H]⁻.

Preparation of N-(2-amino-5-methylphenyl)-2-chloroacetamide (47). A suspension of iron dust (670 mg, 12.0 mmol) in the aqueous solution (15 mL) of ammonium chloride (64 mg, 1.2 mmol) and acetic acid (72 mg, 1.2 mmol) was activated by stirring at 50° C. for 15 min, upon which a solution of ethyl 2-((4-methoxy-2-nitrophenyl)amino)acetate (46) (250 mg, 1.09 mmol) in DMF (3 mL) was added in one portion. After 25 min at 50° C., saturated sodium bicarbonate solution (3 mL) was added and the resulting mixture was filtered through a Celite® pad. The collected solid and Celite® were thoroughly washed with ethyl acetate (30 mL). The organic and aqueous layers of the filtrate were separated and the aqueous layer was extracted with ethyl acetate (3×20 mL). The organic fractions were combined, dried with sodium sulfate and concentrated under reduced pressure to provide N-(2-amino-5-methylphenyl)-2-chloroacetamide (47) (260 mg, quantitative yield) which was used in the next step without further purification.

Preparation of 4-(3-(2,4-dimethyl-6-oxo-1,6-dihydropyrimidin-5-yl)propanoyl)-7-methyl-3,4-dihydroquinoxalin-2(1H)-one, 49. A solution of 3-(2,4-dimethyl-6-oxo-1,6-dihydropyrimidin-5-yl)propanoyl chloride (5), prepared from 3-(2,4-dimethyl-6-oxo-1,6-dihydropyrimidin-5-yl)propanoic acid (4) (50 mg, 0.26 mmol) following the General Procedure 1, in DMF (5 mL) was added in portions to the solution of N-(2-amino-5-methylphenyl)-2-chloroacetamide (47) (0.43 mmol) in DMF (2 mL). After stirring for 15 minutes at room temperature, potassium carbonate (100 mg, 0.72 mmol) was added. After overnight, sodium hydride (60 mg, 1.5 mmol) was added to the mixture and after an additional 20 minutes the suspension was filtered through a Celite® pad. The solid and Celite® were washed with ethyl acetate. The filtrate was quenched with acetic acid (1 mL) and concentrated under reduced pressure. Purification of the product was accomplished by silica gel column chromatography (eluted with a gradient of 0% to 12% methanol-chloroform) provided 49 (15.2 mg, 18% yield) as a white amorphous powder.

¹H NMR (600 MHz, DMSO-d6) δ 12.14 (br s, 1H), 10.59 (s, 1H), 7.29 (br s, 1H), 6.81 (dd, J=1.2, 8.1 Hz, 1H), 6.78 (d, J=1.2 Hz, 1H), 4.30 (s, 2H), 2.66-2.55 (m, 4H), 2.27 (s, 3H), 2.18 (s, 3H), 2.12 (br s, 3H).

LC/MS: Eluent system A (retention time: 4.79 min); ESI-MS 341.2 [M+H]⁺, 339.2 [M−H]⁻, 385.0 [M+HCO₂⁻]⁻.

### Compound 50

### Synthesis of 4-[3-(2-cyclopropyl-4-methyl-6-oxo-1,6-dihydropyrimidin-5-yl)propanoyl]-3,4-dihydroquinoxalin-2(1H)-one, 50

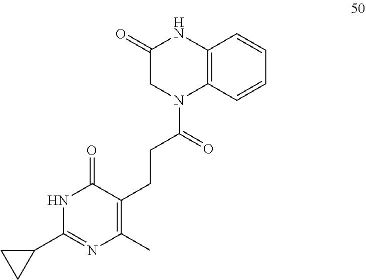 [SMILES: Cc1nc(C2CC2)[nH]c(=O)c1CCC(=O)N1CC(=O)Nc2ccccc21]

Compound 50 was synthesized as in Scheme 19.

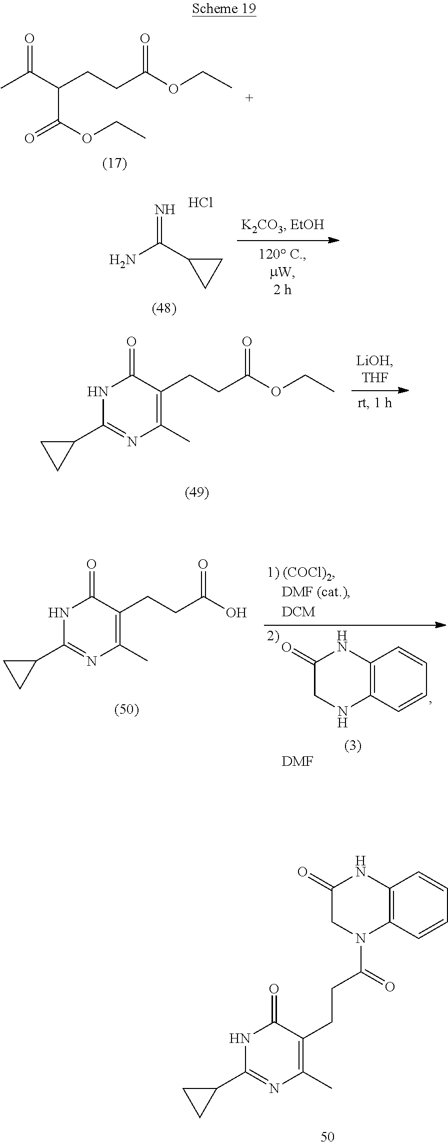

Preparation of ethyl 3-(2-cyclopropyl-4-methyl-6-oxo-1,6-dihydropyrimidin-5-yl)propanoate, (49). To a microwave reaction vessel was added diethyl 2-acetylglutarate (17) (1.8 mL, 8.3 mmol), cyclopropanecarboximidamide hydrochloride (48) (1.0 g, 8.3 mmol), and K₂CO₃ (1.1 g, 8.3 mmol) and absolute ethanol (10 mL). The microwave reactor was set to 120° C. for 2 h. The mixture after cooling to ambient temperature was filtered through a pad of Celite®. To the filtrate was added silica gel (10 g) and the mixture was concentrated under reduced pressure. The silica gel and loaded on column and chromatography was performed with a gradient of 0% to 10% MeOH—CHCl₃, which provided (49) (548 mg, 26% yield) as a white solid.

¹H NMR (600 MHz, CDCl₃) δ 12.02-11.63 (m, 1H), 4.13 (q, J=7.2 Hz, 2H), 2.88-2.73 (m, 2H), 2.59-2.49 (m, 2H), 2.28 (s, 3H), 1.80 (tt, J=4.7, 8.1 Hz, 1H), 1.25 (t, J=7.2 Hz, 3H), 1.19-1.14 (m, 2H), 1.08-1.03 (m, 2H).

LC/MS: Eluent system A (retention time: 5.81 min); ESI-MS: 251.2 [M+H]⁺.

Preparation of 3-(2-cyclopropyl-4-methyl-6-oxo-1,6-dihydropyrimidin-5-yl)propanoic acid (50). To a mixture of the ester (49) (230 mg, 0.92 mmol) in THF (3 mL) at room temperature was added a 1.0 M LiOH (1.8 mL) solution in water. After 2 h, the mixture was treated with 1.0 N HCl solution (1.8 mL). The pH was adjusted to 5-6 with a 1.0 N HCl solution. The volatiles were removed under reduced pressure. The product (50) was used without further purification in the next step.

Preparation of 4-[3-(2-cyclopropyl-4-methyl-6-oxo-1,6-dihydropyrimidin-5-yl)propanoyl]-3,4-dihydroquinoxalin-2(1H)-one, 50. To a suspension of 3-(2-cyclopropyl-4-methyl-6-oxo-1,6-dihydropyrimidin-5-yl)propanoic acid (50) (100 mg, 0.45) in DCM (10 mL) was added oxalyl chloride (76 μL, 0.90 mmol), followed by addition of 1-2 drops of DMF. The mixture was stirred at room temperature for 1 h upon which the solvent was removed under reduced pressure. The residue was co-evaporated with DCM two times (10 mL each) and tried under reduced pressure. To the residue was added anhydrous DMF (3 mL) followed by 3,4-dihydro-1H-quinoxalin-2-one (3) (67 mg, 0.45 mmol). After 30 min, the mixture was concentrated under reduced pressure and the resulting residue was dissolved in MeOH. Silica gel (4 g) was added to absorb the crude product. The silica gel was dried and loaded on a silica gel column. The product was purified by column chromatography with a gradient of 0% to 20% MeOH in EtOAc, which produced 50 (35.3 mg, 22% yield) as a light yellow solid.

¹H NMR (600 MHz, DMSO-d₆) δ 12.36 (br s, 1H), 10.65 (s, 1H), 7.45 (br s, 1H), 7.18 (br t, J=7.7 Hz, 1H), 7.04-6.96 (m, 2H), 4.33 (br s, 2H), 2.67-2.61 (m, 2H), 2.61-2.55 (m, 2H), 2.08 (br s, 3H), 1.81 (br s, 1H), 0.99-0.88 (m, 4H).

LC/MS: Eluent system A (retention time: 5.19 min); ESI-MS: 353.2 [M+H]⁺.

### Compound 51

### Synthesis of 4-[3-(2-ethyl-4-methyl-6-oxo-1,6-dihydropyrimidin-5-yl)propanoyl]-3,4-dihydroquinoxalin-2(1H)-one, 51

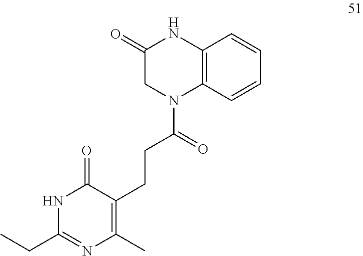 [SMILES: CCc1nc(C)c(CCC(=O)N2CC(=O)Nc3ccccc32)c(=O)[nH]1]

Compound 51 was synthesized as in Scheme 20.

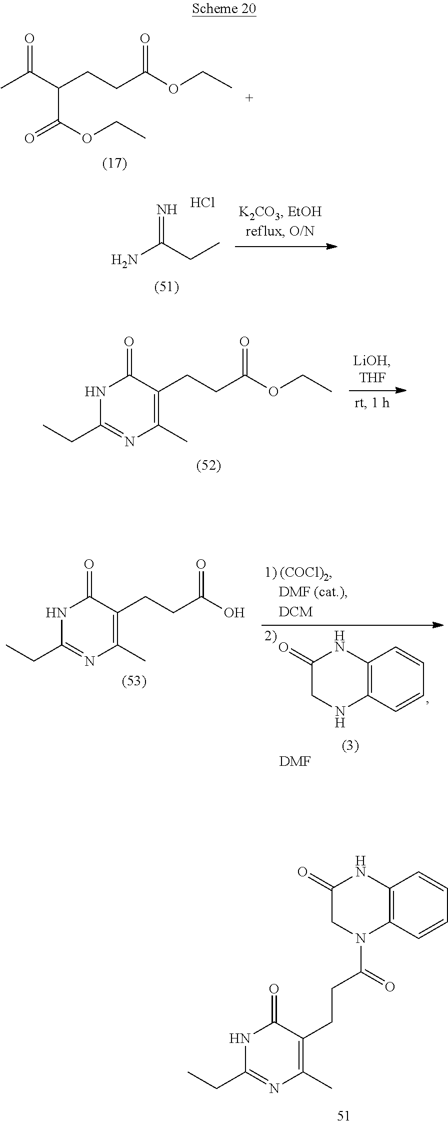

Preparation of ethyl 3-(2-ethyl-4-methyl-6-oxo-1,6-dihydropyrimidin-5-yl)propanoate, (52). To a round-bottomed flask was added diethyl 2-acetylglutarate (17) (2.0 mL, 9.2 mmol), propionamidine hydrochloride (51) (1.0 g, 9.2 mmol), K₂CO₃ (1.3 g, 9.2 mmol) and absolute ethanol (30 mL). The reaction mixture was heated to reflux. After overnight, the mixture was cooled to room temperature, and filtered through a pad of Celite®. The filtrate was added to silica gel and the mixture concentrated under reduced pressure. The silica gel was dried and loaded on a silica gel column. Column chromatography with a gradient of 0% to 10% MeOH/CHCl₃, provided compound (52) (1.2 g, 55% yield) as a white solid.

¹H NMR (600 MHz, CDCl3) δ 12.09 (br s, 1H), 4.15 (q, J=7.2 Hz, 2H), 2.85 (t, J=7.7 Hz, 2H), 2.67 (q, J=7.7 Hz, 2H), 2.61-2.57 (m, 2H), 2.38 (s, 3H), 1.35 (t, J=7.7 Hz, 3H), 1.27 (t, J=7.2 Hz, 3H).

LC/MS: Eluent system A (retention time: 4.55 min); ESI-MS: 239.2 [M+H]⁺.

Preparation of 3-(2-ethyl-4-methyl-6-oxo-1,6-dihydropyrimidin-5-yl)propanoic acid (53). To a mixture of ethyl 3-(2-ethyl-4-methyl-6-oxo-1,6-dihydropyrimidin-5-yl)propanoate (52) (200 mg, 0.84 mmol) in THF (3 mL) at room temperature was added 1.0 M LiOH solution in water (1.7 mL). After 2 h, the mixture was treated with the 1.0 N HCl solution (1.7 mL). The pH was adjusted to 5-6 with 1.0 N HCl solution. The volatiles were removed under reduced pressure. The product (53) was used without further purification in the next step.

Preparation of 4-[3-(2-ethyl-4-methyl-6-oxo-1,6-dihydropyrimidin-5-yl)propanoyl]-3,4-dihydroquinoxalin-2(1H)-one, 51. To a suspension of 3-(2-ethyl-4-methyl-6-oxo-1,6-dihydropyrimidin-5-yl)propanoic acid (53) (100 mg, 0.48) in DCM (10 mL) was added oxalyl chloride (81 μL, 0.96 mmol), followed by addition of 1-2 drops of DMF. After 1 h, the mixture was concentrated under reduced pressure. The residue was co-evaporated with DCM two times (10 mL each). To the dried residue was added anhydrous DMF (5 mL) followed by 3,4-dihydro-1h-quinoxalin-2-one (3) (71 mg, 0.48 mmol). After 30 min, the mixture was concentrated under reduced pressure. The resulting residue was dissolved in methanol, silica gel (4 g) was added and the volatiles were removed under reduced pressure. Column chromatography with a gradient of 0% to 20% MeOH/EtOAc generated 51 (26.3 mg, 16% yield) as a pale yellow solid.

¹H NMR (600 MHz, DMSO-d6) δ 12.13 (br s, 1H), 10.64 (s, 1H), 7.51-7.38 (m, 1H), 7.21-7.12 (m, 1H), 7.04-6.94 (m, 2H), 4.32 (br s, 2H), 2.68-2.62 (m, 2H), 2.62-2.56 (m, 2H), 2.44 (q, J=7.6 Hz, 2H), 2.14 (br s, 3H), 1.13 (t, J=7.6 Hz, 3H).

LC/MS: Eluent system A (retention time: 4.47 min); ESI-MS: 341.3 [M+H]⁺.

### Compound 52

### Synthesis of 4-{3-[4-methyl-6-oxo-2-(propan-2-yl)-1,6-dihydropyrimidin-5-yl]propanoyl}-3,4-dihydroquinoxalin-2(1H)-one, 52

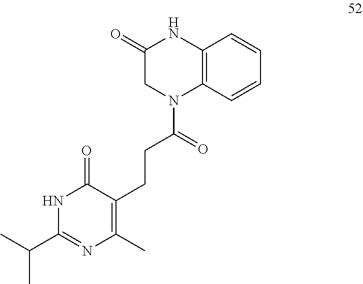 [SMILES: Cc1nc(C(C)C)[nH]c(=O)c1CCC(=O)N1CC(=O)Nc2ccccc21]

Compound 52 was synthesized as in Scheme 21

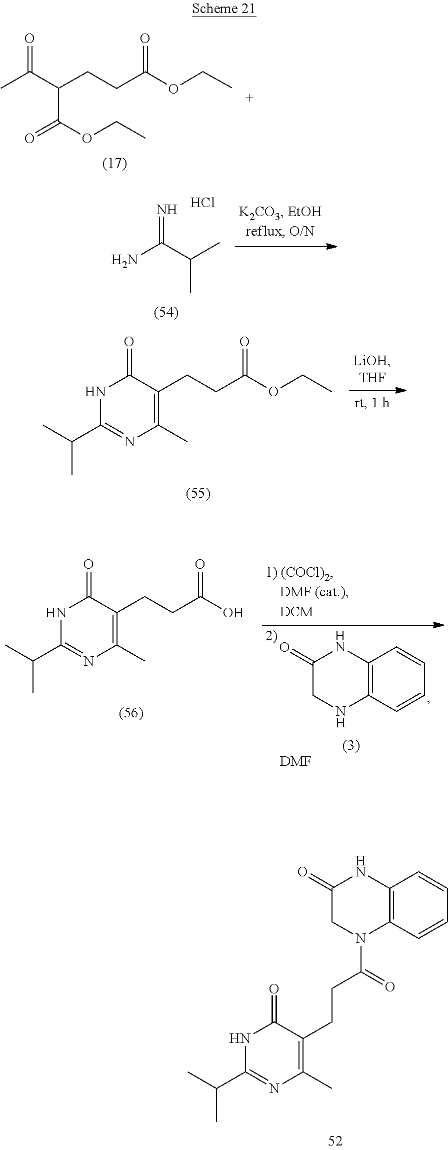

Preparation of ethyl 3-[4-methyl-6-oxo-2-(propan-2-yl)-1,6-dihydropyrimidin-5-yl]propanoate (55). To a round-bottomed flask was added diethyl 2-acetylglutarate (17) (1.8 mL, 8.2 mmol), 2-methylpropanimidamide hydrochloride (54) (1.0 g, 8.2 mmol), K₂CO₃ (1.1 g, 8.2 mmol) and absolute ethanol (30 mL). After heating to reflux overnight, the mixture was cooled and filtered through a pad of Celite®. The filtrate was added to silica gel and the volatiles were removed under reduced pressure. Column chromatography with a gradient of 0% to 10% MeOH—CHCl₃ provided compound (55) (765 mg, 37% yield) as a white solid.

¹H NMR (600 MHz, CDCl₃) δ 11.57 (br s, 1H), 4.14 (q, J=7.2 Hz, 2H), 2.90-2.79 (m, 3H), 2.60-2.55 (m, 2H), 2.36 (s, 3H), 1.33 (d, J=7.0 Hz, 6H), 1.25 (t, J=7.2 Hz, 3H).

LC/MS: Eluent system A (retention time: 5.87 min); ESI-MS: 253.3 [M+H]⁺.

Preparation of 3-[4-methyl-6-oxo-2-(propan-2-yl)-1,6-dihydropyrimidin-5-yl]propanoic acid (56). To a mixture of the ethyl 3-[4-methyl-6-oxo-2-(propan-2-yl)-1,6-dihydropyrimidin-5-yl]propanoate (55) (125 mg, 0.5 mmol) in THF (2 mL) at room temperature was added a 1.0 M LiOH solution (1.0 mL) in water. After 2 h, 1.0 N HCl solution (1.0 mL) was added. The pH was adjusted to 5-6 and the volatiles were removed under reduced pressure. The product (56) was used without further purification in the next step.

Preparation of 4-{3-[4-methyl-6-oxo-2-(propan-2-yl)-1,6-dihydropyrimidin-5-yl]propanoyl}-3,4-dihydroquinoxalin-2(1H)-one, 52. To a suspension of the 3-[4-methyl-6-oxo-2-(propan-2-yl)-1,6-dihydropyrimidin-5-yl]propanoic acid (56) (100 mg, 0.45) in DCM (10 mL) was added oxalyl chloride (76 μL, 0.90 mmol), followed by 1-2 drops of DMF. After 1 h, the mixture was concentrated under reduced pressure. The residue was co-evaporated with DCM two times (10 mL each). The resulting residue was dried under reduced pressure and anhydrous DMF (5 mL) and 3,4-dihydro-1h-quinoxalin-2-one (3) (67 mg, 0.45 mmol) were added. After 30 mins, the mixture was concentrated and the residue was dissolved in methanol. Silica gel (4 g) was added and the mixture was concentrated. After drying the silica gel under reduced pressure it was loaded on a column. Column chromatography with a gradient of 0% to 20% MeOH-EtOAc generated 52 (35.8 mg, 22% yield) as a pale yellow solid.

¹H NMR (600 MHz, DMSO-d₆) δ 12.12 (br s, 1H), 10.65 (s, 1H), 7.46 (br s, 1H), 7.22-7.14 (m, 1H), 7.03-6.97 (m, 2H), 4.34 (br s, 2H), 2.76-2.70 (m, 1H), 2.68-2.57 (m, 4H), 2.16 (br s, 3H), 1.15 (d, J=6.8 Hz, 6H).

LC/MS: Eluent system A (retention time: 5.47 min); ESI-MS: 355.3 [M+H]⁺.

### Compound 53

### Synthesis of N-[2-(dimethylamino)-2-oxoethyl]-3-(4-hydroxy-2,6-dimethylpyrimidin-5-yl)-N-phenylpropanamide, 53

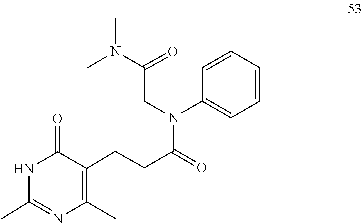 [SMILES: Cc1nc(C)c(CCC(=O)N(CC(=O)N(C)C)c2ccccc2)c(=O)[nH]1]

Compound 53 was synthesized as in Scheme 22.

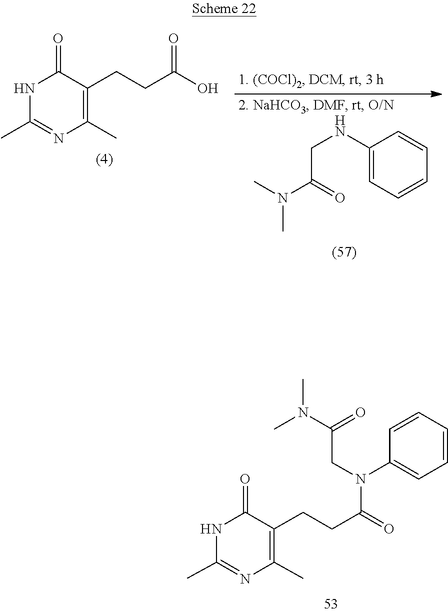 [SMILES: Cc1nc(C)c(CCC(=O)O)c(=O)[nH]1 ; CN(C)C(=O)CNc1ccccc1 ; Cc1nc(C)c(CCC(=O)N(CC(=O)N(C)C)c2ccccc2)c(=O)[nH]1]

Preparation of N-[2-(dimethylamino)-2-oxoethyl]-3-(4-hydroxy-2,6-dimethylpyrimidin-5-yl)-N-phenylpropanamide, 53. To an ice-cooled suspension of 3-(4-hydroxy-2,6-dimethylpyrimidin-5-yl)propanoic acid (4) (75.0 mg, 0.38 mmol) in DCM (25 mL) was added slowly oxalyl chloride (195.4 mg, 1.54 mmol) followed by DMF (0.05 mL). After warming to room temperature and stirring for 2 h, the mixture was concentrated under reduced pressure. The resulting foam was mixed with 2-anilino-N,N-dimethylacetamide (57) (142.5 mg, 0.80 mmol) and NaHCO₃ (84.0 mg, 1.0 mmol) in DMF (3 mL). After overnight, the mixture was concentrated under reduced pressure. The resulting residue was dissolved in chloroform (10 mL), solid removed by filtration and the product purified by column chromatography on silica gel (eluted with a gradient of 0% to 5% methanol in chloroform) generated 53 (36.0 mg, 25% yield) as a gray solid.

¹H NMR (600 MHz, DMSO-d₆) δ 12.12 (br s, 1H), 7.48-7.21 (m, 5H), 4.43 (s, 2H), 2.93 (s, 3H), 2.81 (s, 3H), 2.54-2.51 (m, 2H), 2.19-2.16 (m, 2H), 2.16 (s, 3H), 2.04 (s, 3H).

LC/MS: Eluent system A (retention time: 4.79 min); ESI-MS: 357.3 [M+H]⁺.

### Compound 54

### Synthesis of 4-{3-[2-methyl-6-oxo-4-(trifluoromethyl)-1,6-dihydropyrimidin-5-yl]propanoyl}-3,4-dihydroquinoxalin-2(1H)-one, 54

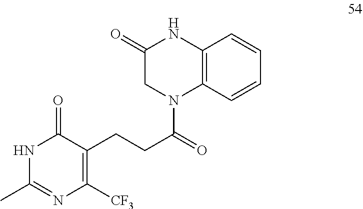 [SMILES: Cc1nc(C(F)(F)F)c(CCC(=O)N2CC(=O)Nc3ccccc32)c(=O)[nH]1]

Compound 54 was synthesized as in Scheme 23.

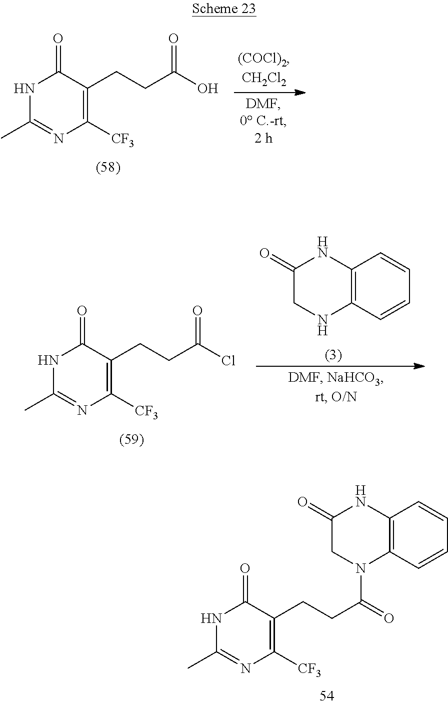 [SMILES: Cc1nc(C(F)(F)F)c(CCC(=O)O)c(=O)[nH]1 ; Cc1nc(C(F)(F)F)c(CCC(=O)Cl)c(=O)[nH]1 ; O=C1CNc2ccccc2N1 ; Cc1nc(C(F)(F)F)c(CCC(=O)N2CC(=O)Nc3ccccc32)c(=O)[nH]1]

Preparation of 4-{3-[2-methyl-6-oxo-4-(trifluoromethyl)-1,6-dihydropyrimidin-5-yl]propanoyl}-3,4-dihydroquinoxalin-2(1H)-one, 54. To an ice-cooled suspension of 3-[2-methyl-6-oxo-4-(trifluoromethyl)-1,6-dihydropyrimidin-5-yl]propanoic acid (58) (200.0 mg, 0.80 mmol) in DCM (25 mL) was added slowly oxalyl chloride (203.0 mg, 1.6 mmol), followed by DMF (0.05 mL). After warming to room temperature and stirring for 2 h, the mixture was concentrated under reduced pressure and dried under high vacuum. The resulting foam (59) (107.4 mg, 0.40 mmol) was mixed with 3,4-dihydroquinoxalin-2(1H)-one (3) (59.3 mg, 0.40 mmol) and NaHCO₃ (33.6 mg, 0.40 mmol) in DMF (3 mL). After overnight, the mixture was concentrated under reduced pressure. The resulting residue was dissolved in chloroform (10 mL), solid removed by filtration and the product purified by column chromatography on silica gel (eluted with a gradient of 0% to 5% methanol in chloroform) produced 54 (19.0 mg, 12% yield) as a gray solid.

¹H NMR (600 MHz, DMSO-d₆) δ 12.94 (br s, 1H), 10.66 (s, 1H), 7.52-7.30 (m, 1H), 7.18 (br t, J=7.2 Hz, 1H), 7.04-6.94 (m, 2H), 4.33 (br s, 2H), 2.81-2.63 (m, 4H), 2.30 (s, 3H). ¹⁹F NMR (564 MHz, DMSO-d₆) δ −63.5 (s, 3F).

LC/MS: Eluent system A (retention time: 5.86 min); ESI-MS: 379.4 [M−H]⁻.

### Compound 55

### Synthesis of 4-[3-(2,4-dimethyl-6-oxo-1,6-dihydropyrimidin-5-yl)propanoyl]-8-fluoro-3,4-dihydroquinoxalin-2(1H)-one, 55

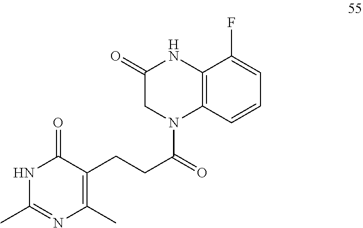 [SMILES: Cc1nc(C)c(CCC(=O)N2CC(=O)Nc3c(F)cccc32)c(=O)[nH]1]

Preparation of 4-[3-(2,4-dimethyl-6-oxo-1,6-dihydropyrimidin-5-yl)propanoyl]-8-fluoro-3,4-dihydroquinoxalin-2(1H)-one, 55. A similar procedure as described for Compound 43 was followed: with 3-(2,4-dimethyl-6-oxo-1,6-dihydropyrimidin-5-yl)propanoic acid (4) (50 mg, 0.25 mmol), oxalyl chloride (0.22 mL, 2.5 mmol), N,N-dimethylformamide (2 drops), dichloromethane (10 mL), and 8-fluoro-3,4-dihydroquinoxalin-2(1H)-one (60) (66 mg, 0.40 mmol) in N,N-dimethylformamide (5 mL). Purification of the product was accomplished by silica gel column chromatography (eluted with a gradient of 0% to 20% methanol-chloroform) afforded 55 (58.6 mg, 67% yield) as an off-white colored solid.

¹H NMR (600 MHz, DMSO-d₆) δ 12.17 (br s, 1H), 10.79 (s, 1H), 7.42-7.23 (m, 1H), 7.16-7.08 (m, 1H), 7.06-6.99 (m, 1H), 4.35 (s, 2H), 2.73-2.64 (m, 2H), 2.64-2.57 (m, 2H), 2.18 (s, 3H), 2.13 (br s, 3H). ¹⁹F NMR (565 MHz, DMSO-d₆) δ −129.00 (br s, 1F).

LC/MS: Eluent system C (retention time: 4.70 min); ESI-MS: 345.3 [M+H]⁺.

### Compound 56

### Synthesis of 4-[3-(2,4-dimethyl-6-oxo-1,6-dihydropyrimidin-5-yl)propanoyl]-6,7-difluoro-3,4-dihydroquinoxalin-2(1H)-one, 56

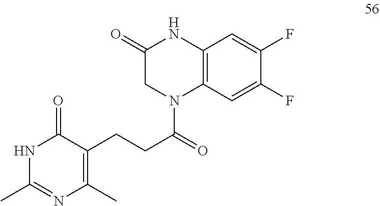 [SMILES: Cc1nc(C)c(CCC(=O)N2CC(=O)Nc3cc(F)c(F)cc32)c(=O)[nH]1]

Preparation of 4-[3-(2,4-dimethyl-6-oxo-1,6-dihydropyrimidin-5-yl)propanoyl]-6,7-difluoro-3,4-dihydroquinoxalin-2(1H)-one, 56. A similar procedure as was described for Compound 43 was followed with: 3-(2,4-dimethyl-6-oxo-1,6-dihydropyrimidin-5-yl)propanoic acid, (4) (30 mg, 0.15 mmol), oxalyl chloride (0.13 mL, 1.5 mmol), N,N-dimethylformamide (2 drops), dichloromethane (10 mL), and 6,7-difluoro-3,4-dihydroquinoxalin-2(1H)-one (61) (45.1 mg, 0.24 mmol) in N,N-dimethylformamide (5 mL). The product was purified by silica gel column chromatography (eluted with a gradient of 0% to 20% methanol-chloroform), which afforded 56 (27.6 mg, 50% yield) as an off-white color solid.

¹H NMR (600 MHz, DMSO-d₆) δ 12.19 (br s, 1H), 10.69 (br s, 1H), 7.73 (dd, J=7.9, 11.1 Hz, 1H), 6.95 (dd, J=7.9, 11.1 Hz, 1H), 4.32 (s, 2H), 2.69-2.64 (m, 2H), 2.64-2.56 (m, 2H), 2.19 (s, 3H), 2.13 (br s, 3H). ¹⁹F NMR (565 MHz, DMSO-d₆) δ −140.02 (br d, J=23 Hz, 1F), −144.83 (br d, J=23 Hz, 1F).

LC/MS: Eluent system C (retention time: 5.73 min); ESI-MS: 363.2 [M+H]⁺.

### Compound 57

### Synthesis of 4-[3-(2,4-dimethyl-6-oxo-1,6-dihydropyrimidin-5-yl)propanoyl]-7-(trifluoromethyl)-3,4-dihydroquinoxalin-2(1H)-one, 57

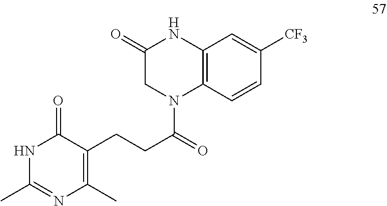 [SMILES: Cc1nc(C)c(CCC(=O)N2CC(=O)Nc3cc(C(F)(F)F)ccc32)c(=O)[nH]1]

Preparation of 4-[3-(2,4-dimethyl-6-oxo-1,6-dihydropyrimidin-5-yl)propanoyl]-7-(trifluoromethyl)-3,4-dihydroquinoxalin-2(1H)-one, 57. A similar procedure as described for Compound 43 was followed with: 3-(2,4-dimethyl-6-oxo-1,6-dihydropyrimidin-5-yl)propanoic acid, (9) (30 mg, 0.15 mmol), oxalyl chloride (0.13 mL, 1.5 mmol), N,N-dimethylformamide (2 drops), dichloromethane (10 mL), and 7-(trifluoromethyl)-3,4-dihydroquinoxalin-2(1H)-one (62) (72.7 mg, 0.34 mmol) in N,N-dimethylformamide (5 mL). The product was purified by silica gel column chromatography (eluted with a gradient of 0% to 10% methanol-chloroform), which afforded 57 (31.8 mg, 53% yield) as an off-white colored solid.

¹H NMR (600 MHz, DMSO-d₆) δ 12.19 (br s, 1H), 10.88 (s, 1H), 7.83-7.66 (m, 1H), 7.36 (dd, J=8.4, 1.6 Hz, 1H), 7.28 (d, J=1.9 Hz, 1H), 4.37 (s, 2H), 2.73-2.67 (m, 2H), 2.65-2.59 (m, 2H), 2.19 (s, 3H), 2.13 (br s, 3H). ¹⁹F NMR (565 MHz, DMSO-d₆) δ −60.94 (s, 3F).

LC/MS: Eluent system A (retention time: 5.77 min); ESI-MS: 395.3 [M+H]⁺.

### Compound 58

### Synthesis of 8-fluoro-4-{3-[2-methyl-6-oxo-4-(trifluoromethyl)-1,6-dihydropyrimidin-5-yl]propanoyl}-3,4-dihydroquinoxalin-2(1H)-one, 58

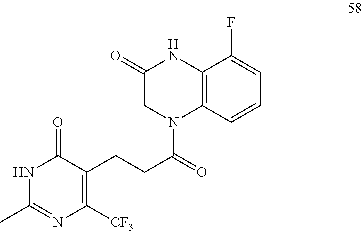 [SMILES: Cc1nc(C(F)(F)F)c(CCC(=O)N2CC(=O)Nc3c(F)cccc32)c(=O)[nH]1]

Compound 58 was synthesized as in Scheme 24.

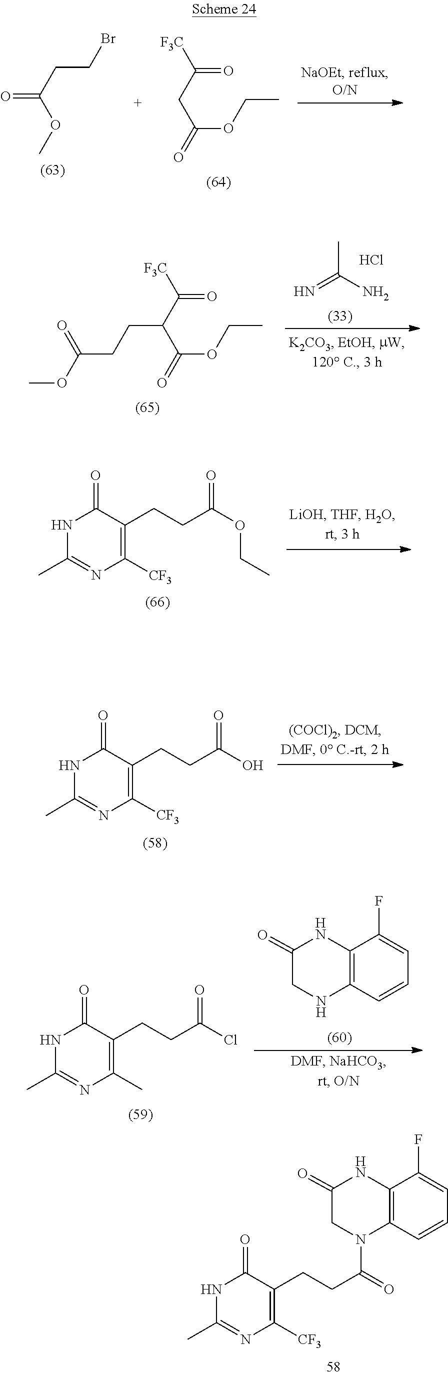

Preparation of 1-ethyl 5-methyl 2-(trifluoroacetyl)pentanedioate, (65). To a solution of sodium ethoxide (27.2 mmol; prepared in situ, from 0.625 g of sodium and 100 mL of ethanol) at room temperature was added ethyl trifluoroacetoacetate (64) (5.0 g, 27.2 mmol). After 10 min, methyl 3-bromopropionate (63) (4.53 g, 27.2 mmol) was added. The resulting mixture was heated to reflux. After overnight the mixture was concentrated under reduced pressure, diluted with ether (100 mL) and washed with water (3×25 mL). The organic layer was dried over magnesium sulfate, filtered, concentrated under reduced pressure and purified by column chromatography on silica gel (eluted with a gradient from 0% to 25% ethyl acetate in hexanes), which generated 65 (3.2 g, 43% yield) as a colorless thick oil. This material was used in the next step without further purification.

LC/MS: Eluent system B (retention time: 7.42 min); ESI-MS: 271.1 [M+H]⁺.

Preparation of ethyl 3-[2-methyl-6-oxo-4-(trifluoromethyl)-1,6-dihydropyrimidin-5-yl]propanoate (66). A mixture of 1-ethyl 5-methyl 2-(trifluoroacetyl)pentanedioate (65) (1.0 g, 3.7 mmol), methylamidine hydrochloride (33) (0.35 g, 3.7 mmol) and potassium carbonate (1.0 g, 7.4 mmol) in ethanol (15 mL) was placed in a microwave reactor that was set to 120° C. for 3 h and then after cooling to ambient temperature was filtered, concentrated under reduced pressure and the residue was dissolved in chloroform (2 mL). The product was purified by column chromatography on silica gel (eluted with a gradient of 0% to 2.5% methanol in chloroform) produced (66) (0.56 g, 54% yield) as a colorless oil. This material was used in the next step without further purification.

LC/MS: Eluent system B (retention time: 5.79 min); ESI-MS: 279.2 [M+H]⁺.

Preparation of 3-[2-methyl-6-oxo-4-(trifluoromethyl)-1,6-dihydropyrimidin-5-yl]propanoic acid (58). To a solution of ethyl 3-[2-methyl-6-oxo-4-(trifluoromethyl)-1,6-dihydropyrimidin-5-yl]propanoate (66) (0.56 g, 2.0 mmol) in THF (10 mL) at room temperature was added lithium hydroxide (239.7 mg, 10.0 mmol, in 1 mL of water). After 3 h, the solution was concentrated under reduced pressure, neutralized to pH ˜ 7 with 1N HCl. The resulting mixture was concentrated under reduced pressure, dissolved in chloroform (25 mL) and filtered. The product was purified by column chromatography on silica (eluted with a gradient of 5% to 20% methanol in chloroform) produced (58) as a colorless semi-solid (400 mg, 80% yield). This material was used without further purification in the next step.

LC/MS: Eluent system B (retention time: 1.98 min); ESI-MS: 251.1 [M+H]⁺.

Preparation of 8-fluoro-4-{3-[2-methyl-6-oxo-4-(trifluoromethyl)-1,6-dihydropyrimidin-5-yl]propanoyl}-3,4-dihydroquinoxalin-2(1H)-one, 58. To an ice cooled suspension of 3-[2-methyl-6-oxo-4-(trifluoromethyl)-1,6-dihydropyrimidin-5-yl]propanoic acid (58) (200.0 mg, 0.8 mmol) in DCM (25 mL) was added slowly oxalyl chloride (203.0 mg, 1.6 mmol), followed by DMF (0.05 mL). After warming to room temperature and stirring for 2 h, the mixture was concentrated under reduced pressure and dried under reduced pressure. The resulting foam (59) (107.4 mg, 0.40 mmol) was mixed with 8-fluoro-3,4-dihydroquinoxalin-2(1H)-one (60) (86.5 mg, 0.40 mmol) and NaHCO₃ (33.6 mg, 0.40 mmol) in DMF (3 mL). After overnight, the mixture was concentrated under reduced pressure. The resulting residue was dissolved in chloroform (10 mL), solid removed by filtration and the product purified by column chromatography on silica gel (eluted with a gradient of 0% to 5% methanol in chloroform) produced 58 (10.0 mg, 6% yield) as a gray solid.

¹H NMR (600 MHz, DMSO-d₆) δ 13.02-12.91 (m, 1H), 10.81 (s, 1H), 7.40-7.27 (m, 1H), 7.17-7.09 (m, 1H), 7.06-6.99 (m, 1H), 4.36 (s, 2H), 2.80-2.66 (m, 4H), 2.30 (s, 3H). ¹⁹F NMR (564 MHz, DMSO-d₆) δ −63.3 (s, 3F), −128.5 (broad, 1F).

LC/MS: Eluent system A (retention time: 5.95 min); ESI-MS: 397.2 [M−H]⁻.

### Compound 59

### Synthesis of 4-[3-(4-ethyl-2-methyl-6-oxo-1,6-dihydropyrimidin-5-yl)propanoyl]-3,4-dihydroquinoxalin-2(1H)-one, 59

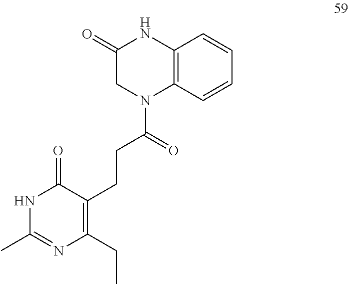 [SMILES: CCc1nc(C)[nH]c(=O)c1CCC(=O)N1CC(=O)Nc2ccccc21]

Compound 59 was synthesis as in Scheme 25.

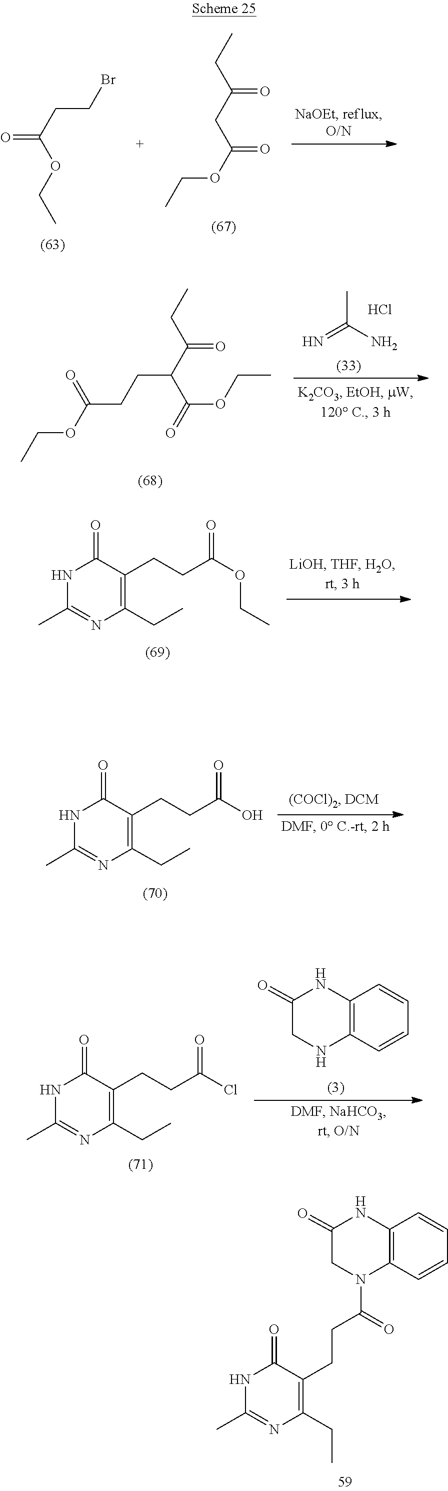

Preparation of 1,5-diethyl 2-propanoylpentanedioate, (68). To a solution of sodium ethoxide (20.2 mmol; prepared in situ, from 0.46 g of sodium and 50 mL of ethanol) at room temperature was added ethyl 3-oxopentanoate (67) (3.0 g, 20.0 mmol). After 10 min, ethyl 3-bromopropionate (63) (3.6 g, 20.0 mmol) was added. The resulting mixture was heated to reflux. After overnight, the mixture was concentrated under reduced pressure, diluted with ether (100 mL) and washed with water (3×50 mL). The organics were dried over magnesium sulfate, filtered and concentrated under reduced pressure and the product purified by column chromatography on silica gel (eluted with a gradient of 0% to 25% ethyl acetate in hexanes) generated (68) (1.3 g, 28% yield) as a colorless thick oil. This material was used without further purification in the next step.

LC/MS: Eluent system B (retention time: 8.49 min); ESI-MS: 245.1 [M+H]⁺.

Preparation of ethyl 3-(4-ethyl-2-methyl-6-oxo-1,6-dihydropyrimidin-5-yl)propanoate, (69). A mixture of 1,5-diethyl 2-propanoylpentanedioate (68) (1.3 g, 5.6 mmol), methylamidine hydrochloride (33) (0.53 g, 5.6 mmol) and potassium carbonate (1.54 g, 11.2 mmol) in ethanol (20 mL) was placed in a microwave reactor that was set to 120° C. for 3 h and after cooling was filtered, concentrated under reduced pressure and the residue dissolved in chloroform (2 mL). The product was purified by column chromatography on silica gel (eluted with a gradient of 0% to 2.5% methanol in chloroform) produced (69) (0.56 g, 54% yield) as a colorless thick oil. This material was used without further purification in the next step.

Preparation of 3-(4-ethyl-2-methyl-6-oxo-1,6-dihydropyrimidin-5-yl)propanoic acid, (70). To a solution of ethyl 3-(4-ethyl-2-methyl-6-oxo-1,6-dihydropyrimidin-5-yl)propanoate (69) (0.39 g, 1.65 mmol) in THF (10 mL) at room temperature was added lithium hydroxide (197.7 mg, 8.25 mmol, in 1 mL of water). After 3 h, the mixture was concentrated under reduced pressure, neutralized to pH ˜ 7 with 1N HCl. The resulting mixture was concentrated under reduced pressure, dissolved in chloroform (25 mL) and filtered. The product was purified by column chromatography on silica (eluted with a gradient of 0%-5% methanol-chloroform) generated (70) (206.0 mg, 59% yield) as a colorless gum. This material was used without further purification in the next step.

LC/MS: Eluent system B (retention time: 1.09 min); ESI-MS: 211.1 [M+H]⁺.

Preparation of 4-[3-(4-ethyl-2-methyl-6-oxo-1,6-dihydropyrimidin-5-yl)propanoyl]-3,4-dihydroquinoxalin-2(1H)-one, 59. To an ice cooled suspension of 3-(4-ethyl-2-methyl-6-oxo-1,6-dihydropyrimidin-5-yl)propanoic acid (70) (206.0 mg, 0.98 mmol) in DCM (25 mL) was added slowly oxalyl chloride (150.0 mg, 1.18 mmol) followed by DMF (0.05 mL). After warming to room temperature and stirring for 2 h, the mixture was concentrated under reduced pressure. The resulting foam (71) was mixed with 3,4-dihydroquinoxalin-2(1H)-one (3) (145.0 mg, 0.98 mmol) and NaHCO₃ (84.0 mg, 0.98 mmol) in DMF (3 mL). After overnight, the mixture was concentrated under reduced pressure. The resulting residue was dissolved in chloroform (10 mL), solid removed by filtration and the product purified by column chromatography on silica gel (eluted with a gradient of 0% to 5% methanol in chloroform) producing 59 (43.0 mg, 13% yield) as a gray solid.

¹H NMR (600 MHz, DMSO-d₆) δ 12.15 (br s, 1H), 10.65 (s, 1H), 7.51-7.38 (m, 1H), 7.23-7.16 (m, 1H), 7.05-6.96 (m, 2H), 4.33 (s, 2H), 2.68-2.56 (m, 4H), 2.44-2.34 (m, 2H), 2.19 (s, 3H), 1.05 (br s, 3H).

LC/MS: Eluent system A (retention time: 4.45 min); ESI-MS: 341.3 [M+H]⁺.

### Example 2: Testing of Compounds

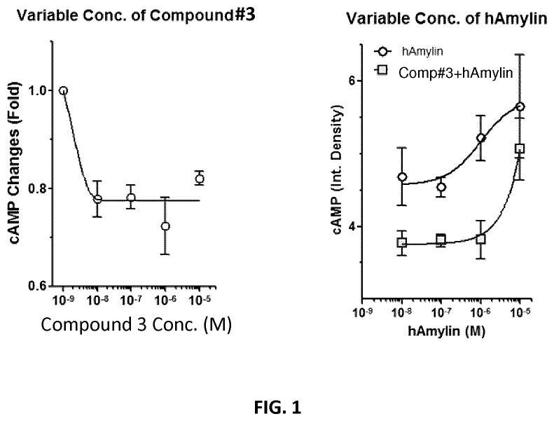

An initial set of 10 compounds was screened using a cyclic AMP (cyclic adenosinemonophosphate, cAMP) assay in amylin receptor subtype 3 expressing cells. Screening of the initial set of 10 compounds using a cAMP assay in amylin receptor subtype 3 expressing cells yielded Compound 3 as an amylin receptor antagonist. See FIG. 1.

Cell Cultures. Amylin receptor subtypes (AMY3-HEK cells) stably expressed in human embryonic kidney (HEK293) cell-line were generated and characterized as described in a previous published article from our laboratory (Fu et al., J. Biol. Chem. 2012). The AMY3-HEK cells were grown in a 5% CO₂ humidified incubator at 37° C. with DMEM, 10% FBS, and 100 μg/mL Zeocin medium.

Two cAMP detection methods were used to validate the findings.

ELISA (enzyme-linked immunosorbent assay). Cellular cAMP levels were measured using a parameter cyclic AMP assay kit (R&D Systems) according to the manufacturer's instructions. Briefly, AMY3-HEK cells were plated on 24-well plates overnight. These cells were then incubated with or without the assay compounds and hAmylin for 5 min. The cells were lysed with lysis buffer provided in the assay kit. Standard curves were plotted using the cAMP standards provided in the ELISA kits. All samples were analyzed in duplicate. The plate is measured at 450 nm. Data was plotted, and non-linear regression was fitted with four parameters using Prism software (GraphPad Software, La Jolla, Calif.).

In-cell Western blot technique. Intracellular cAMP signaling profiles were also determined using in-cell Western blot technique as previously described (Fu W, et al., J Biol Chem. 2012; 287(22):18820-30). AMY3-HEK cells were seeded at 10,000 cells/well in a 96-well plate (Nalge Nunc Intl., Rochester, N.Y.) in DMEM, 10% FBS, Zeocin medium and cultured overnight. Then these cells were incubated with assay compounds or amylin receptor antagonists (AC253,R⁵) and hAmylin for 5 min. Subsequently, cells were fixed with 4% paraformaldehyde for 20 min, permeabilized with 0.2% Triton X-100 PBS solution, blocked with Odyssey blocking buffer (LI-COR, Lincoln, Nebr.), and stained with the following target antibodies. For cyclic adenosinemonophosphate (cAMP) quantification, mouse monoclonal anti-cAMP (R&D Systems) was used as a primary antibody, and IRDye 800 goat anti mouse antibody (LI-COR) was used as a secondary antibody, whereas Sapphire700 and DRAQ5 were used for cell number normalization (LI-COR). Plates were imaged using an Odyssey Infrared Imaging System (LI-COR), and the integrated intensity was normalized to the total cell number on the same well.

Compounds were ranked based on potency of reduction (at 10 μM) of 1 μM human Amylin (hAmylin) induced cAMP increases.

### Example 3: Compound 3 Reduces Human Amylin and Amyloid Beta Induced Cytotoxicity in Neuronal Cells

Effect of Compound 3 on neuronal cells was tested using two different cell death and proliferation assays.

MTT Cell Death Assay. The N2a (mouse) and SK-N-SH (human) neuronal cells were seeded to 5000 cells/well in a 96-well plate in DMEM medium, 10% FBS and incubated overnight. Cells in culture medium were incubated either with or without the assay compounds and Aβ₁₋₄₂, 10 μM for 24-48 h. At the end of treatment, 20 μL of 5 mg/mL 3-(4,5-dimethylthiazol-2-yl)-2,5-diphenyltetrazolium bromide (MTT, Sigma) was added to each well and incubated at 37° C. for 3 h. Medium was removed, 100 μL of MTT solvent (isopropanol with 4 mM HCl) was added to each well, and the plates were incubated for 30 min at room temperature on a rotating shaker. Plates were analyzed on a microplate reader at a 562 nm wavelength.

Live/Dead cell assay. LIVE/DEAD™ Viability/Cytotoxicity Kit from ThermoFisher Scientific (Invitrogen) was used according to the instructions of the manufacturer. Briefly, The N2a cells were cultured in 8 well Lab-Tek chamber slide (ThermoFisher Scientific, Nunc). Cells were incubated either with or without the assay compounds, followed by treatment with Aβ₁₋₄₂ for 24 h. At the end of treatment, the cells were washed three times with D-PBS, followed added live/dead reagent in D-PBS and culture for 30 min. Then fixed the cells with 4% paraformaldehyde for 10 min. Further washed the cells with PBS three times and sealed with coverslip. View the labeled cells under fluorescence microscope. Fluorescent microscopy images were acquired with an Axioplan-2 fluorescence microscope with AxioVision software (Carl Zeiss Ltd., Toronto, ON, Canada). Live/Dead cells were analyzed with Image J software (Schneider, C. A., Rasband, W. S., Eliceiri, K. W. “NIH Image to ImageJ: 25 years of image analysis”. Nature Methods 2012; 9:671-675).

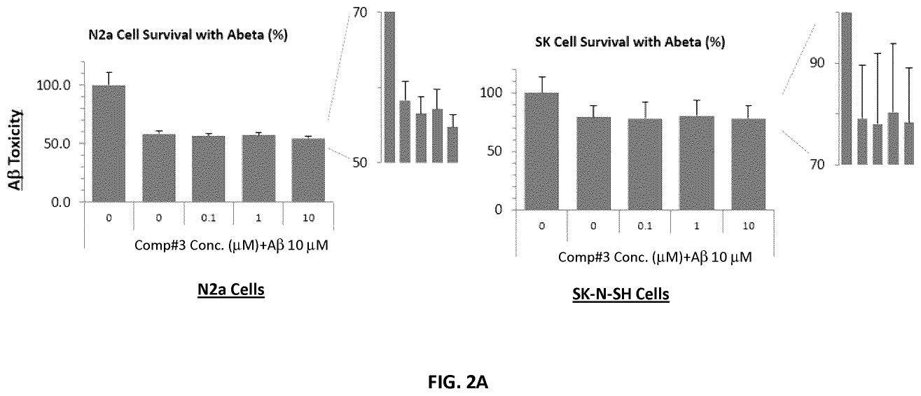

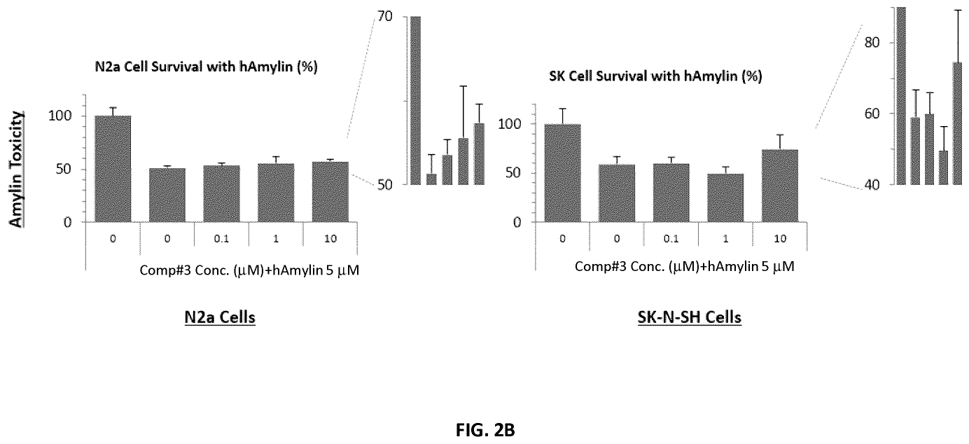

FIGS. 2A and 2B. In mouse and human neuronal cell lines co-application of Compound 3 blunted human amylin and amyloid beta induced cytotoxicity. Neuronal cell lines N2a and SK-N-HS were treated with increasing concentrations of Compound 3 plus 10 μM Aβ₁₋₄₂ or 5 μM hAmylin for 48 h.

### Example 4: Compound 3 Increases Hippocampal Long Term Potentiation (LTP)

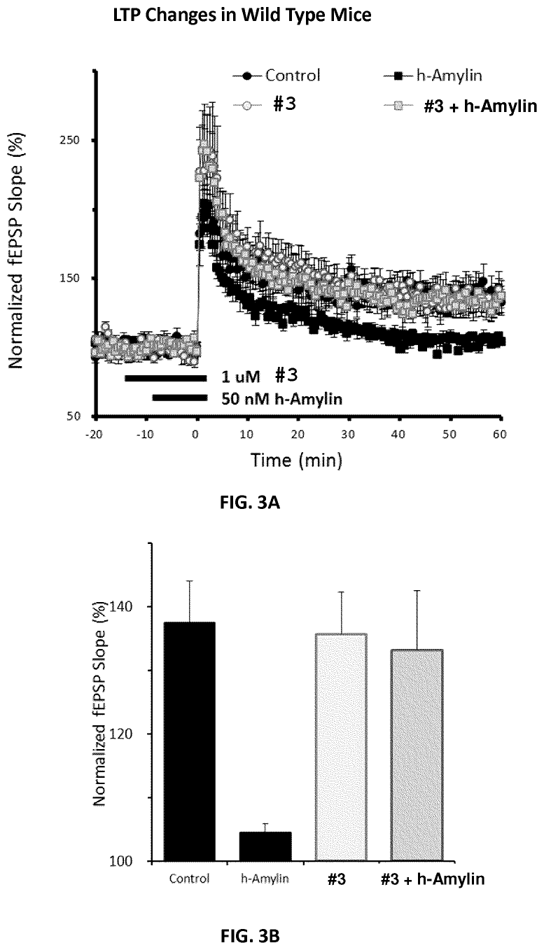

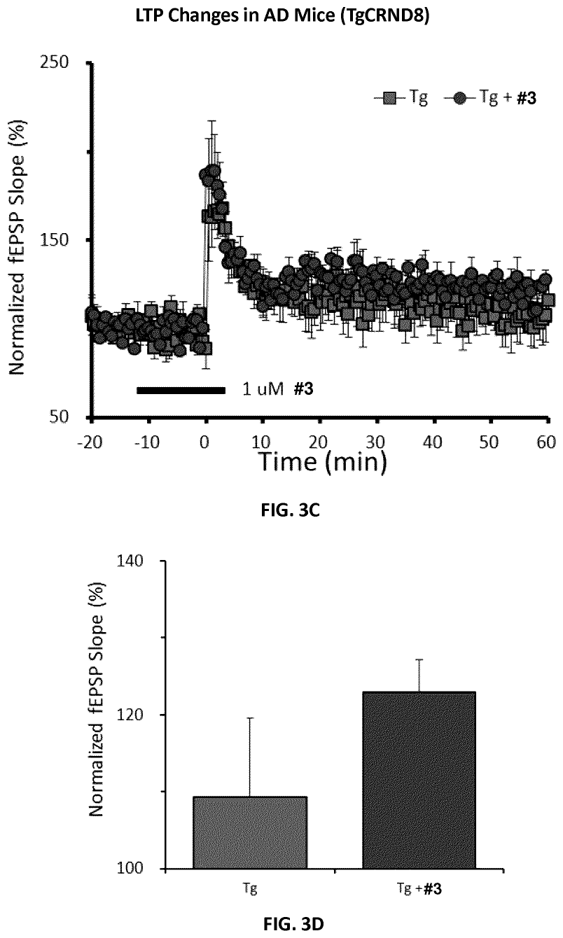

LTP is a cellular surrogate of memory. In brain hippocampal slices, Compound 3 application at 1 μM blocks human amylin-induced depression of LTP (FIGS. 3A and 3B). In hippocampal brain slices from transgenic AD mice (TgCRND8), LTP is chronically depressed. Application of Compound 3 increases LTP levels (FIGS. 3C and 3D) to those seen in age matched control mice (not shown).

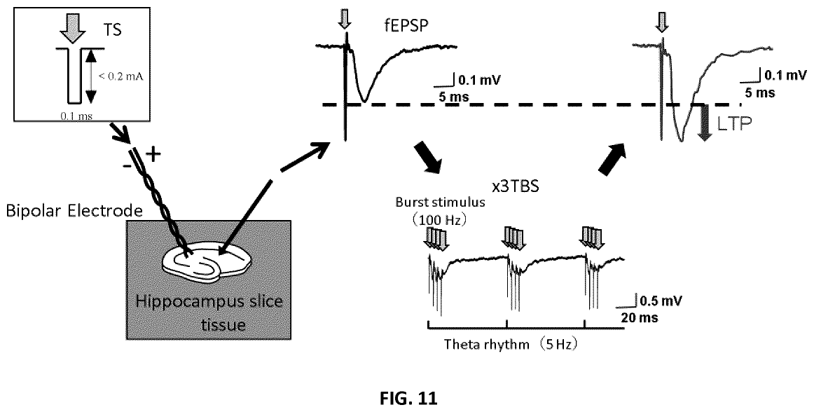

Hippocampal long term potentiation (LTP) electrophysiology experiments: an in vitro cellular surrogate for memory. Brains were quickly removed from mice following decapitation, placed in a cold artificial cerebral spinal fluid (aCSF) on a vibratome chamber and transverse sections cut through the hippocampus. The aCSF contained (in mM) 124 NaCl, 3 KCl, 2.4 CaCl₂), 2 MgCl₂, 1.25 NaH₂PO₄, 26 NaHCO₃ and 10 D-glucose, and was equilibrated with 95% O₂ and 5% CO₂. Hippocampal slices (400 m thick) were maintained in aCSF-filled holding chamber at room temperature for at least 1 hour and individually transferred to the submerged glass bottom recording chamber, which was constantly perfused with aCSF (2 mL/min) at 30° C. Field excitatory postsynaptic potential (fEPSP) was recorded with a metallic (Pt/Ir) electrode (FHC, Bowdoin, Me.) from the stratum radiatum layer of Cornu ammonis 1 region of the hippocampus (CA1) area, and the Schaffer collateral afferents were stimulated with 100-μs test pulses via a bipolar cluster electrode (FHC) (Kimura et al., 2012, Kimura et al. 2016). To evaluate basal synaptic transmission, we applied different stimulation strengths (75 μA to 300 μA in steps of 25 μA) and plotted the amplitudes of presynaptic fiber volleys versus the corresponding fEPSP slopes to compare the slope of input/output (I/O) curves of fEPSP. For long-term potentiation (LTP) experiments, the stimulus strength was set to elicit 40-50% of the maximum fEPSP amplitude and test pulses were delivered to Schaffer collaterals once every 30 seconds. LTP was induced by 3-theta-burst stimulation (3-TBS) protocol (each burst consisted of 4 pulses at 100 Hz with a 200-ms inter-burst interval). Before 3-TBS or drug application, the responses were monitored for at least 10 minutes to ensure a stable baseline of fEPSP. To determine whether the magnitude of LTP differed significantly between groups, average responses during the last 20-min block of recordings (40-60 min after TBS) were compared. Results were from various treatment groups were plotted as histograms with means±standard error (SE). Statistical analysis was performed using one-way ANOVA followed by post-hoc Tukey's honestly significant difference (HSD) test (for multiple comparisons) or Student's t test (for pair-wise comparisons). All drugs and chemicals were applied directly to the slice via bath perfusion, which allowed for a complete exchange of the perfusate in less than a minute and a half A schematic of the LTP electrophysiology assay is shown in FIG. 11.

FIGS. 3A-3D. LTP is a cellular surrogate of memory. In brain hippocampal slices from wild type mice, Compound 3 application at 1 μM blocks human amylin-induced depression of LTP (FIGS. 3A-3B). In hippocampal brain slices from transgenic AD mice (TgCRND8), LTP is chronically depressed. Application of Compound 3 increases LTP levels (FIGS. 3C-3D) to those seen in age matched control mice.

### Example 5: Compound 3 Analogues

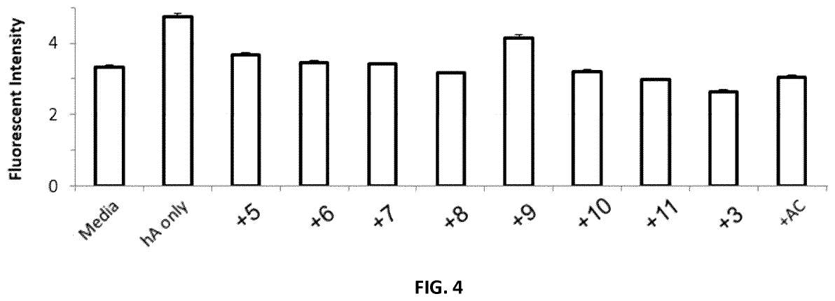

Compound 3 analogues 5 to 11 were ordered from Enamine, Ltd. and tested in cAMP assay (100 nM hAmylin and 10 μM Compound 3 analogs). Changes in cAMP levels indicated that none of the compounds were more potent than Compound 3. See FIG. 4.

### Example 6: Testing of Resynthesized Compound 3 (3IH)

Compound 3 was resynthesized in house and was designated as 3IH. In-house synthesis offered greater purity and less probability of contamination along with fresher batch of the compound.

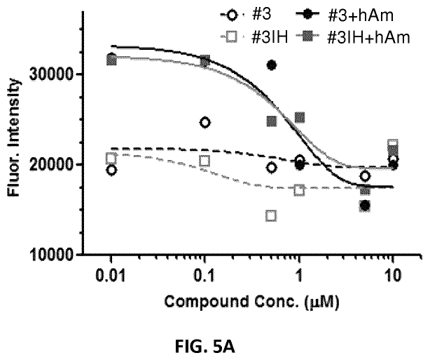

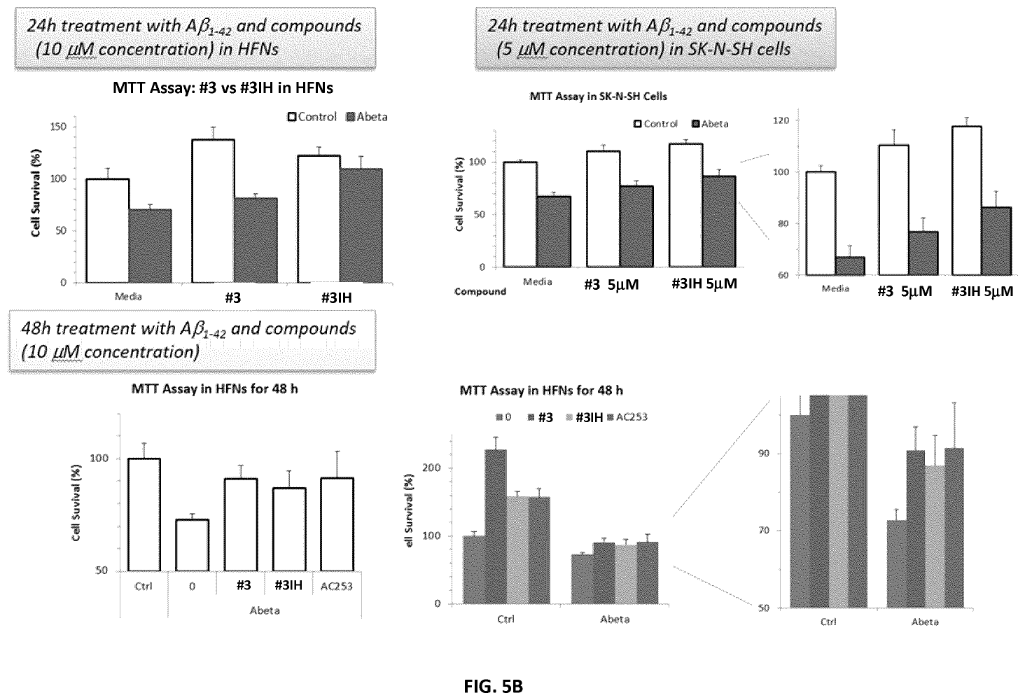

Compound 3IH produced effects identical to those seen with Compound 3 in blocking human amylin (hAM) generated cAMP responses. FIG. 5A. In cytotoxicity assays using human neuronal cell line (SK-N-SH) and primary cultures of human fetal neurons (HFNs), both Compound 3IH and Compound 3 demonstrate identical neuroprotective effects. FIG. 5B.

### Example 7: Compound 3IH Analogues

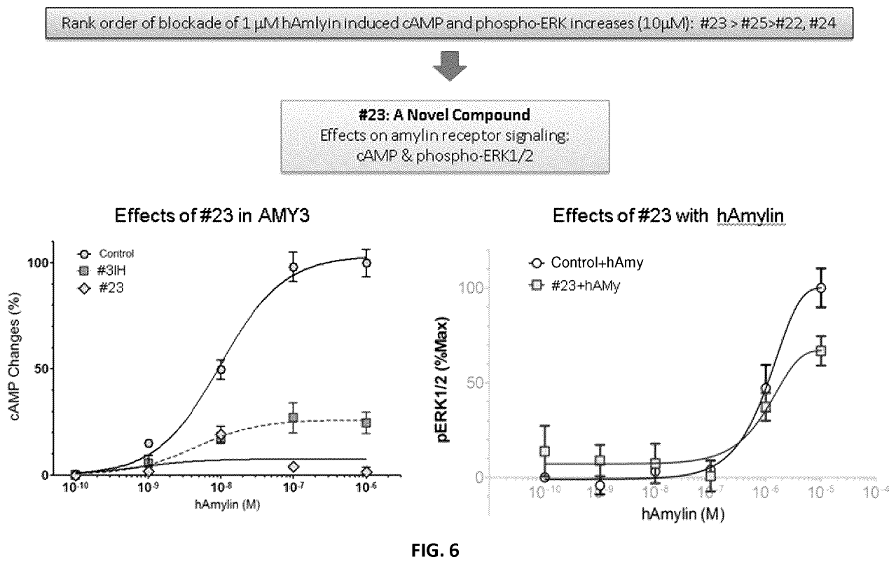

Analogues of Compound 3IH, Compounds 11-21 were available from Enamine library. Four additional analogues Compounds 22-25 were designed based on Compound 3. Compound 23 was identified as most potent of these four analogues based on cAMP assay and downstream phosphoERK response. FIG. 6.

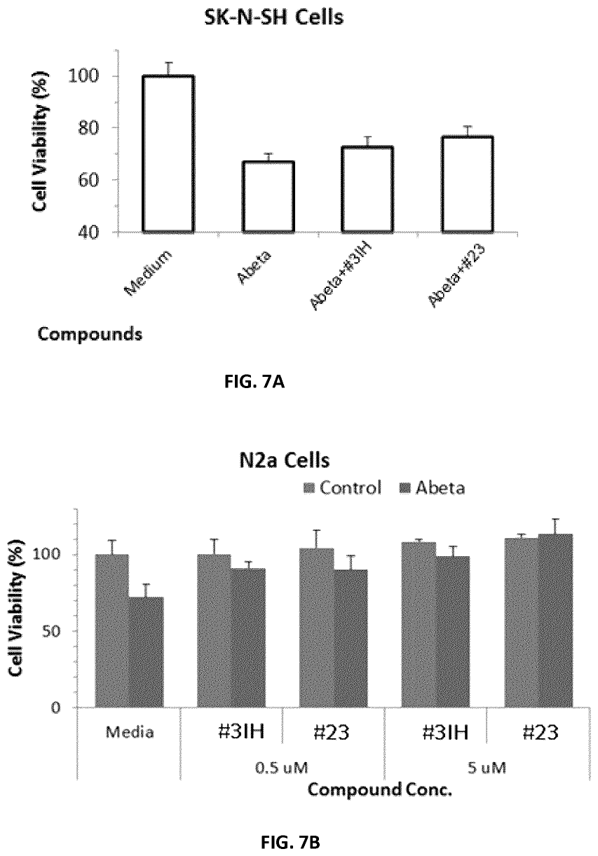

Compound 23 is neuroprotective against amyloid beta toxicity in mouse and human neuronal cell lines. FIG. 7A. SK-N-SH cells were exposed to 10 μM Aβ₁₋₄₂ for 24 hours, in presence of Compound 14 or Compound 23. FIG. 7B. N2a cells were exposed to 10 μM Aβ₁₋₄₂ for 24 hours, in presence of Compound 14 or Compound 23.

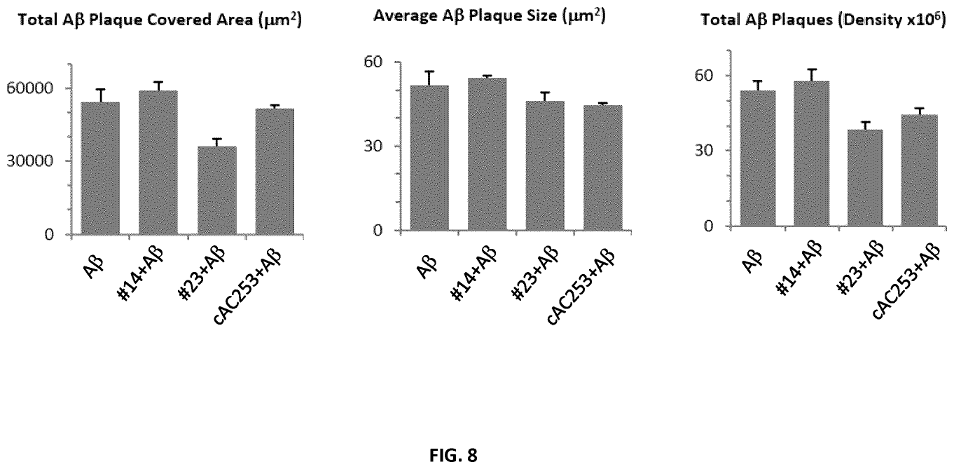

Compound 23 and cyclized AC253 but not Compound 14 creases total Aβ plaques and the area covered by plaques. See FIG. 8.

Live/Dead cell assay confirmed neuroprotective effects of Compound 23 against Aβ toxicity. Data not shown.

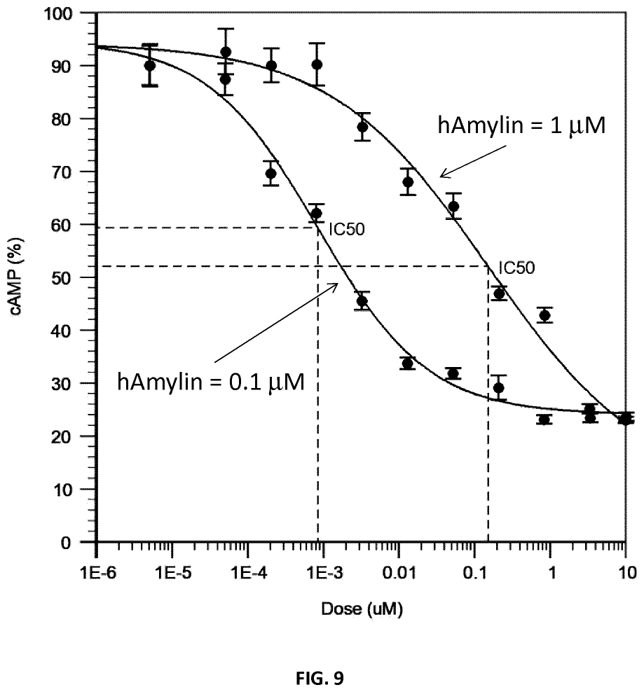

FIG. 9 shows dose-response relationship of Compound 23 against human amylin (at two concentrations) generated cAMP responses. Compound 23 and human amylin were applied simultaneously. Compound 23 IC₅₀ required to block 0.1 μM hAmylin induced cAMP increase is 0.001 μM. Compound 23 IC₅₀ required to block 1 μM hAmylin induced cAMP increase is 0.150 μM.

### Example 8: Testing of Compounds

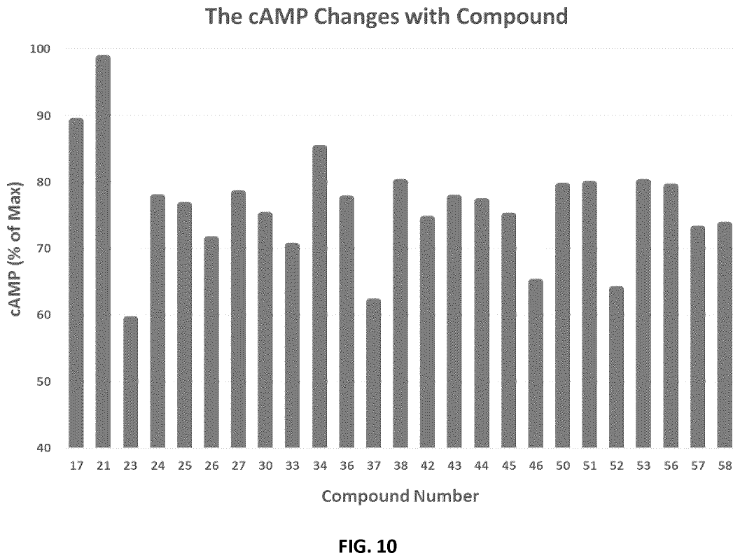

Additional compounds that were synthesized according to Example 1 were also screened using cyclic AMP (cyclic adenosinemonophosphate, cAMP) assay in amylin receptor subtype 3 expressing cells, as described in Example 2. Data are shown in the graph in FIG. 10.

### Example 9: Hippocampal Long Term Potentiation (LTP) Electrophysiology Experiments

Compound 23 was tested in a hippocampal long term potentiation (LTP) electrophysiology assay, as described in Example 4.

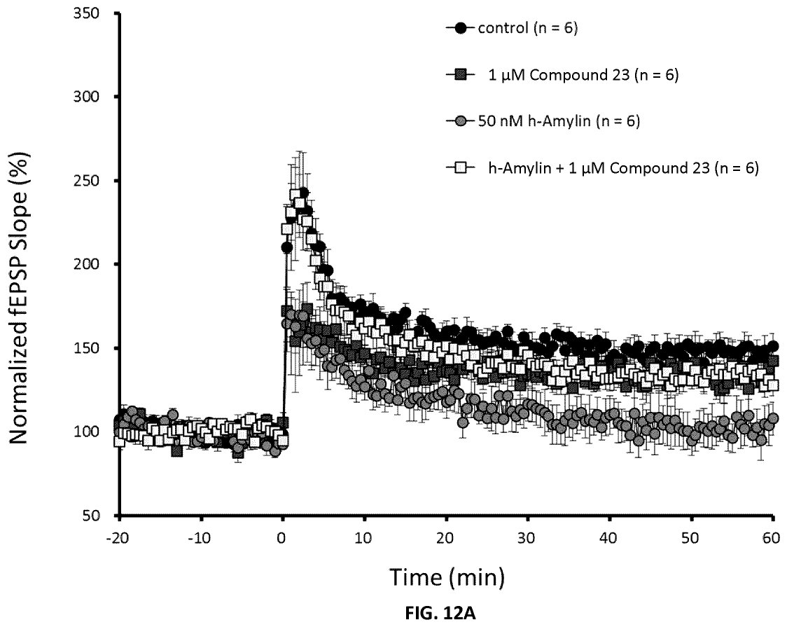

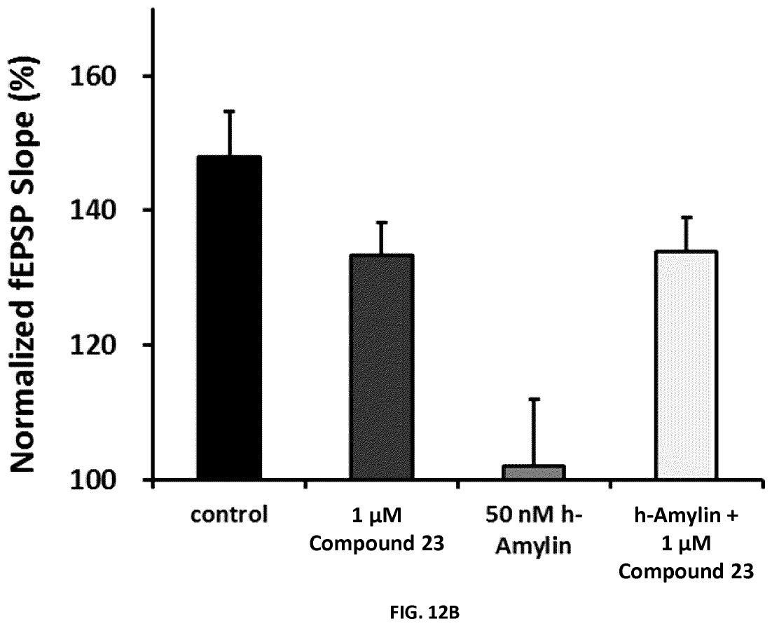

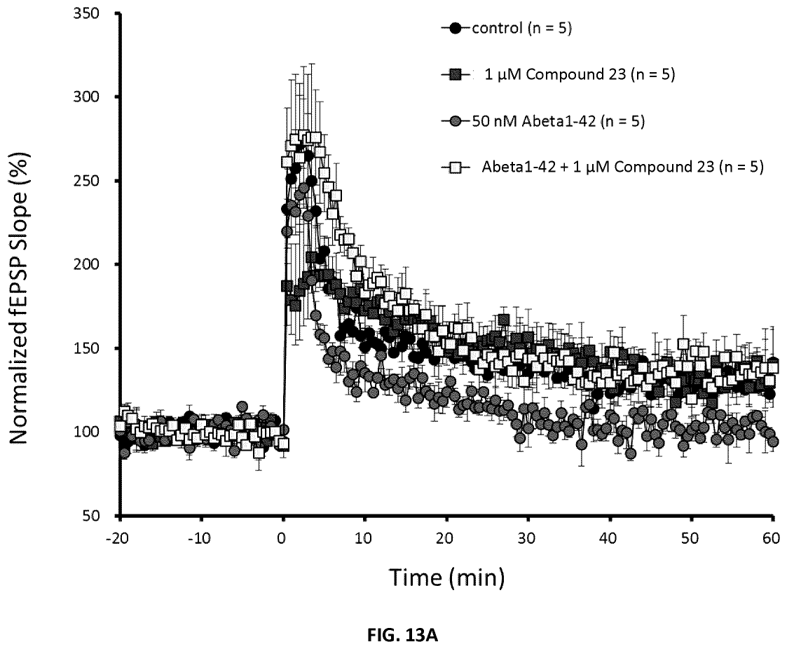

FIGS. 12A-12B. In a hippocampal LTP electrophysiology assay, Compound 23 at 1 μM restored the reduction in LTP by nanomolar dose of human amylim (h-Amylin) to control levels (FIG. 12A). Compound 23 blocked human amylin effects on LTP (n=6 in each group) (FIG. 12B).

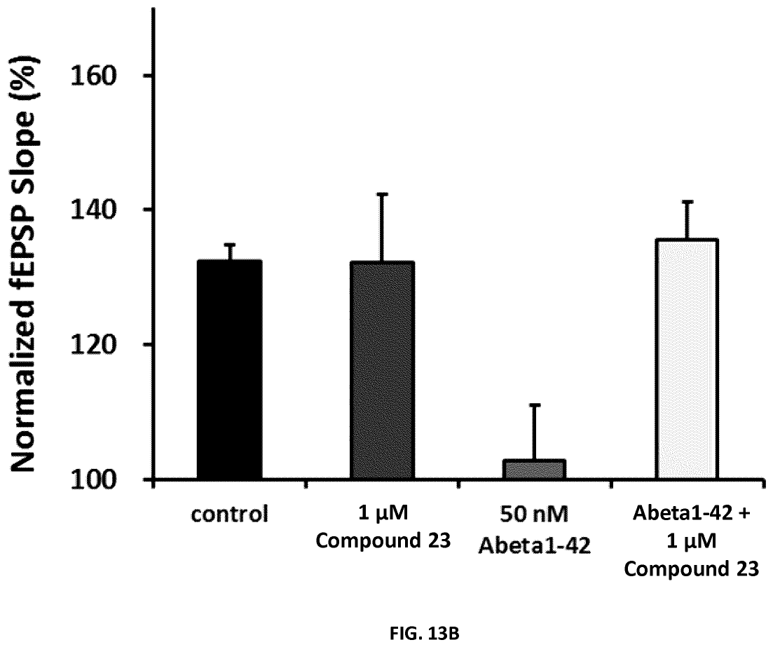

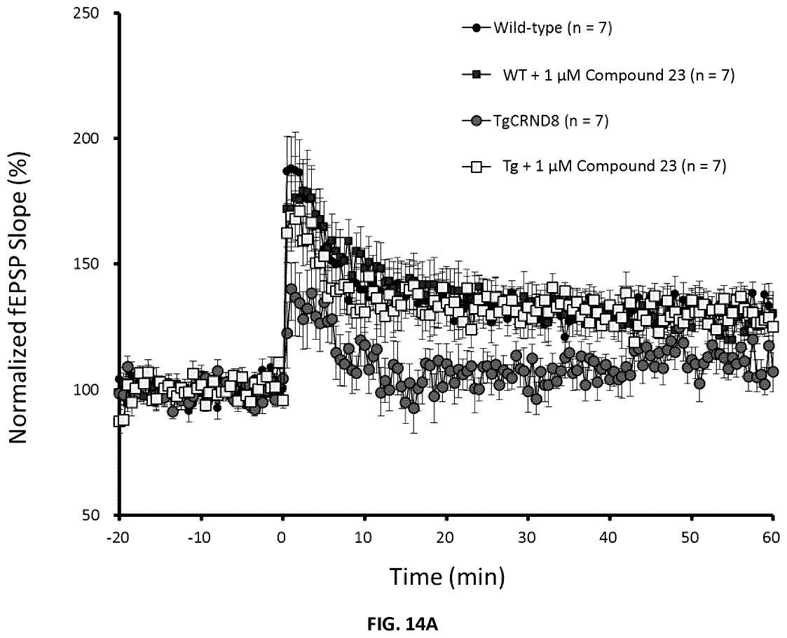

FIGS. 13A-13B. The reduction in LTP caused by nanomolar dose of amyloid beta (Aβ) was restored to control levels by 1 μM Compound 23 (n=5 in each group) (FIG. 13A). FIG. 13B shows a graph of composite data showing Compound 23 blocked amyloid beta (Aβ) effects on LTP (n=6 in each group).

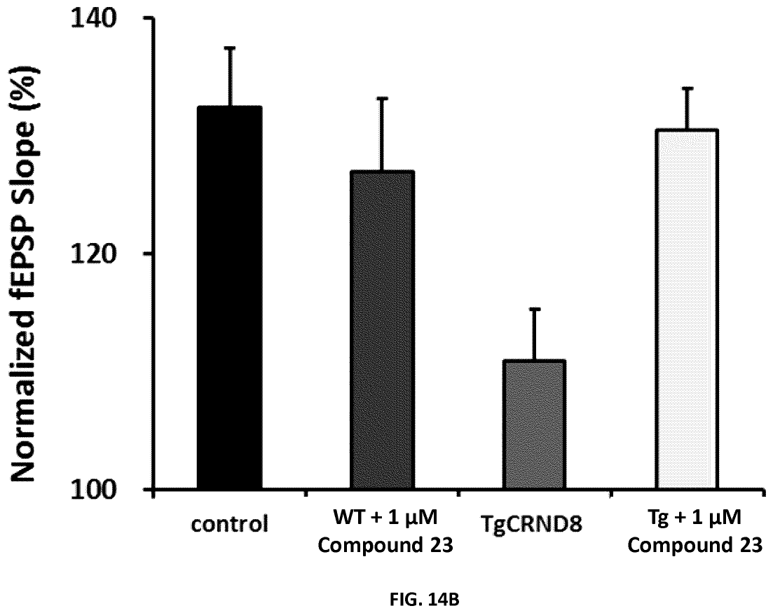

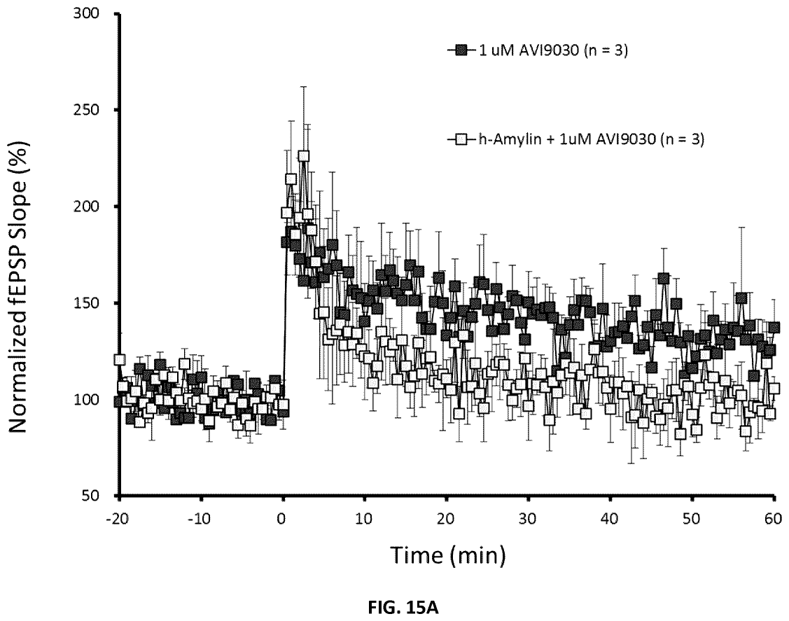

FIGS. 14A-14B. In aged (8 months+) transgenic AD mice (TgCRND8) low levels of basal LTP were restored to levels comparable to those seen in age-matched wild type (WT) littermate control mice (n=7 for each group) (FIG. 14A). FIG. 14B shows a graph of composite data showing Compound 23 restoration of LTP in AD mice to levels comparable to wild type mice (n=6 in each group).

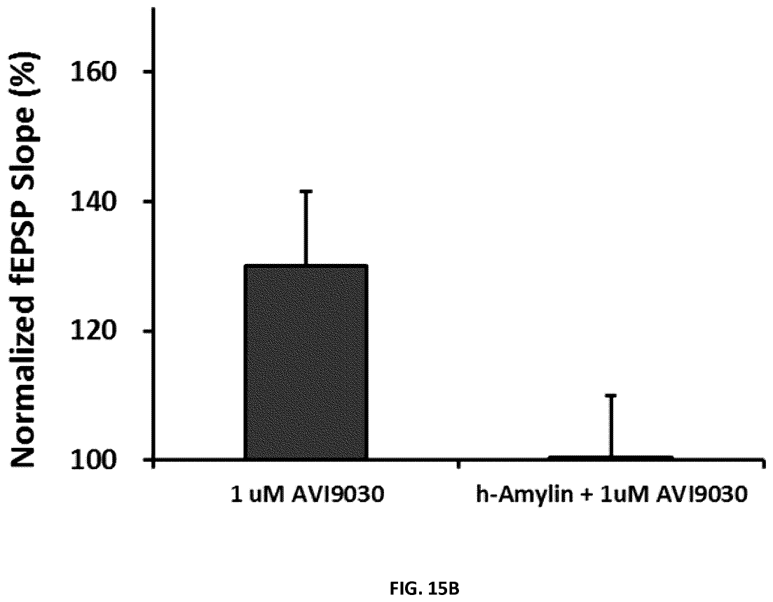

FIGS. 15A-15B. An inactive compound (AVI9030; methyl N-[(1S)-2-methyl-1-[[(2S)-2-(5-phenyl-1H-imidazol-2-yl)-1-pyrrolidinyl]carbonyl]propyl]carbamate) did not block human amylin-induced reduction of LTP (FIG. 15A). FIG. 15B shows a graph of composite data showing an inactive compound was unable to block of human amylin effects on LTP (n=6 in each group).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inhibiting activity of an amylin receptor, the method comprising:
administering to a subject in need thereof, a therapeutically effective amount of a compound of formula (I): 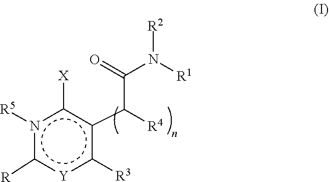
wherein:
R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;
R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² comprise a heterocycle, substituted heterocycle, fused-heterocycle or substituted fused-heterocycle;
R³ is selected from the group consisting of C₁-C₆-alkyl, substituted C₁-C₆-alkyl, C₃-C₆-cycloalkyl, substituted C₃-C₆-cycloalkyl, —CF₃, phenyl, and substituted phenyl;
each R⁴ is independently selected from the group consisting of —H and —CH₃;
R⁵ is present or absent, and if present is selected from the group consisting of —H and —CH₃;
each R⁶ is independently selected from the group consisting of —H, —CH₃, and —CH₂CH₃;
n is an integer from 1 to 3;
X is selected from the group consisting of ═O, ═NH, and —OCH₃;
Y is selected from the group consisting of —N═ and —CH═; and
or an enantiomer, a mixture of enantiomers, a mixture of two or more diastereomers, a tautomer, a mixture of two or more tautomers, or an isotopic variant thereof;
or a pharmaceutically acceptable salt, solvate, or hydrate thereof.

2. The method of claim 1, wherein the administering is effective for reducing cyclic AMP signal production in a cell.

3. The method of claim 1, wherein the amylin receptor is an AMY3 receptor.

4. The method of claim 1, wherein the administering is effective for producing a neuroprotective effect against amylin and/or amyloid-beta protein induced neurotoxicity.

5. The method of claim 1, wherein the administering is effective for treating a disease mediated through activity of the amylin receptor.

6. The method of claim 5, wherein the disease is Alzheimer's disease.

7. The method of claim 1, wherein the compound is of formula (II): 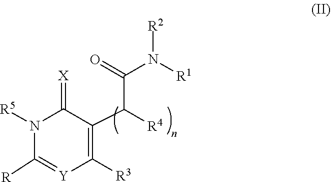
wherein:
R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;
R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle;
R³ is selected from the group consisting of C₁-C₆-alkyl, substituted C₁-C₆-alkyl, C₃-C₆-cycloalkyl, substituted C₃-C₆-cycloalkyl, —CF₃, phenyl, and substituted phenyl;
each R⁴ is independently selected from the group consisting of —H and —CH₃;
R⁵ is selected from the group consisting of —H and —CH₃;
each R⁶ is independently selected from the group consisting of —H, —CH₃, and —CH₂CH₃;
n is an integer from 1 to 3;
X is selected from the group consisting of ═O, and ═NH;
Y is selected from the group consisting of —N═ and —CH═; and
or an enantiomer, a mixture of enantiomers, a mixture of two or more diastereomers, a tautomer, a mixture of two or more tautomers, or an isotopic variant thereof;
or a pharmaceutically acceptable salt, solvate, or hydrate thereof.

8. The method of claim 1, wherein the compound is of formula (III): 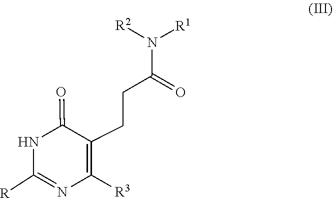
wherein:
R is selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, —NHC(═O)R⁶, —N(R⁶)₂, —OR⁶, aryl, substituted aryl, heteroaryl, substituted heteroaryl, and alkylthio;
R¹ and R² are each independently selected from the group consisting of —H, C₁-C₆-alkyl, substituted C₁-C₆-alkyl, C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle, or together R¹ and R² can comprise a heterocycle, substituted heterocycle, fused-heterocycle or substituted fused-heterocycle;
R³ is selected from the group consisting of C₁-C₆-alkyl, substituted C₁-C₆-alkyl, C₃-C₆-cycloalkyl, substituted C₃-C₆-cycloalkyl, and —CF₃; and
each R⁶ is independently selected from the group consisting of —H, —CH₃, and —CH₂CH₃;
or an enantiomer, a mixture of enantiomers, a mixture of two or more diastereomers, a tautomer, a mixture of two or more tautomers, or an isotopic variant thereof;
or a pharmaceutically acceptable salt, solvate, or hydrate thereof.

9. The method of claim 8, wherein R is heterocyclyl or substituted heterocyclyl.

10. The method of claim 8, wherein R¹ is —H or —CH₃.

11. The method of claim 8, wherein R² is selected from the group consisting of C₃-C₆-cycloalkyl, substituted C₃-C₆-cycloalkyl, heterocyclyl, substituted heterocyclyl, aryl, substituted aryl, heteroaryl, substituted heteroaryl, fused-heterocycle, and substituted fused-heterocycle.

12. The method of claim 8, wherein R¹ and R² together comprise a heterocycle, substituted heterocycle, fused-heterocycle or substituted fused-heterocycle.

13. The method of claim 8, wherein R³ is —CH₃ or —CF₃.

14. The method of claim 1, wherein the compound is selected from the group consisting of

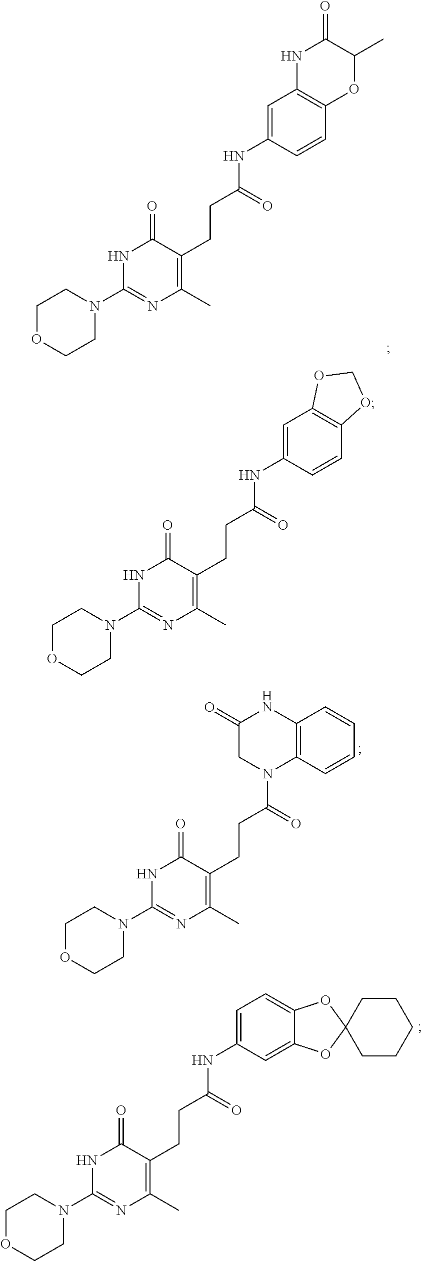 [SMILES: Cc1nc(N2CCOCC2)[nH]c(=O)c1CCC(=O)Nc1ccc2c(c1)NC(=O)C(C)O2 ; Cc1nc(N2CCOCC2)[nH]c(=O)c1CCC(=O)Nc1ccc2c(c1)OCO2 ; Cc1nc(N2CCOCC2)[nH]c(=O)c1CCC(=O)N1CC(=O)Nc2ccccc21 ; Cc1nc(N2CCOCC2)[nH]c(=O)c1CCC(=O)Nc1ccc2c(c1)OC1(CCCCC1)O2]

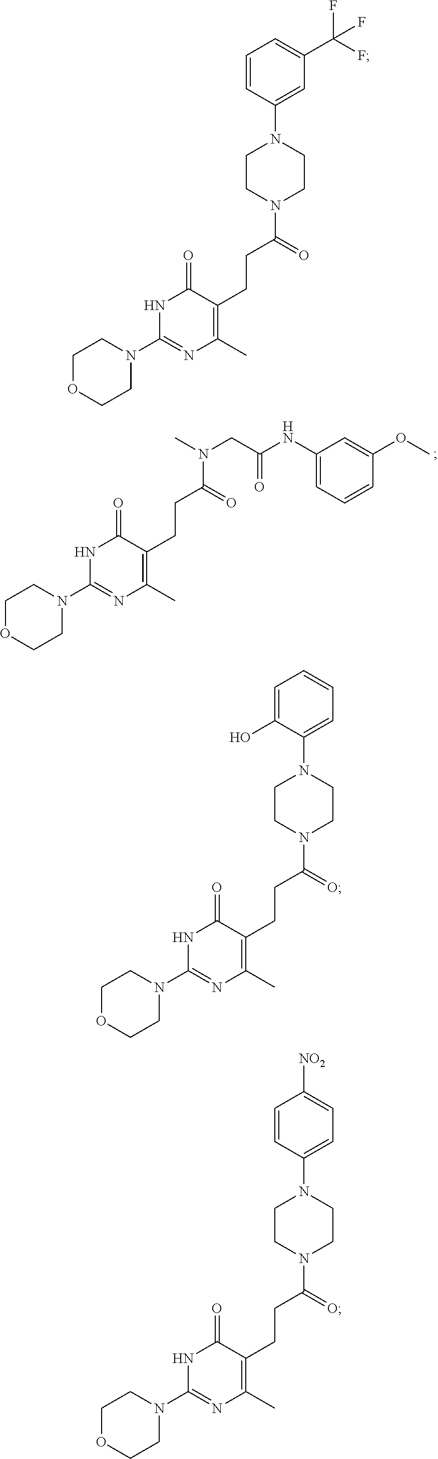 [SMILES: Cc1nc(N2CCOCC2)[nH]c(=O)c1CCC(=O)N1CCN(c2cccc(C(F)(F)F)c2)CC1 ; COc1cccc(NC(=O)CN(C)C(=O)CCc2c(C)nc(N3CCOCC3)[nH]c2=O)c1 ; Cc1nc(N2CCOCC2)[nH]c(=O)c1CCC(=O)N1CCN(c2ccccc2O)CC1 ; Cc1nc(N2CCOCC2)[nH]c(=O)c1CCC(=O)N1CCN(c2ccc([N+](=O)[O-])cc2)CC1]

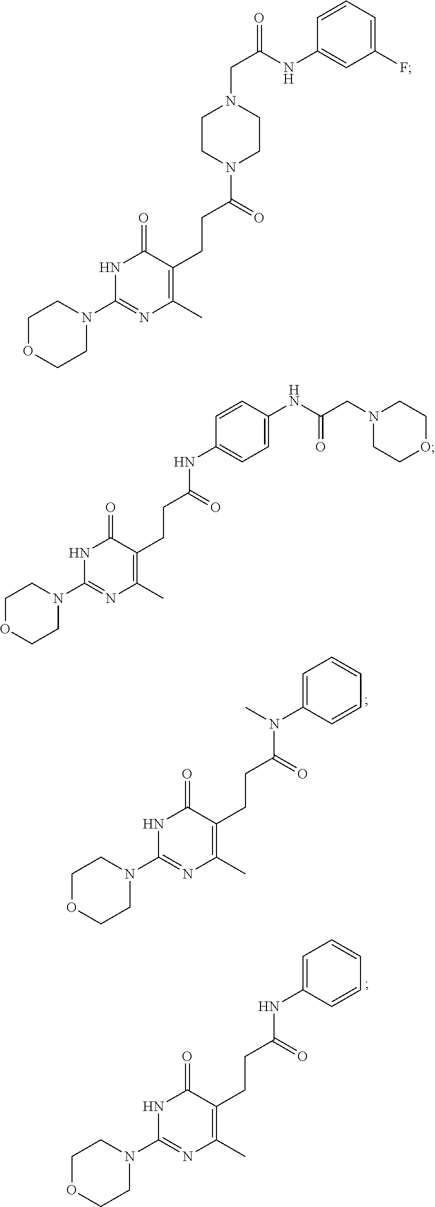 [SMILES: Cc1nc(N2CCOCC2)[nH]c(=O)c1CCC(=O)N1CCN(CC(=O)Nc2cccc(F)c2)CC1 ; Cc1nc(N2CCOCC2)[nH]c(=O)c1CCC(=O)Nc1ccc(NC(=O)CN2CCOCC2)cc1 ; Cc1nc(N2CCOCC2)[nH]c(=O)c1CCC(=O)N(C)c1ccccc1 ; Cc1nc(N2CCOCC2)[nH]c(=O)c1CCC(=O)Nc1ccccc1]

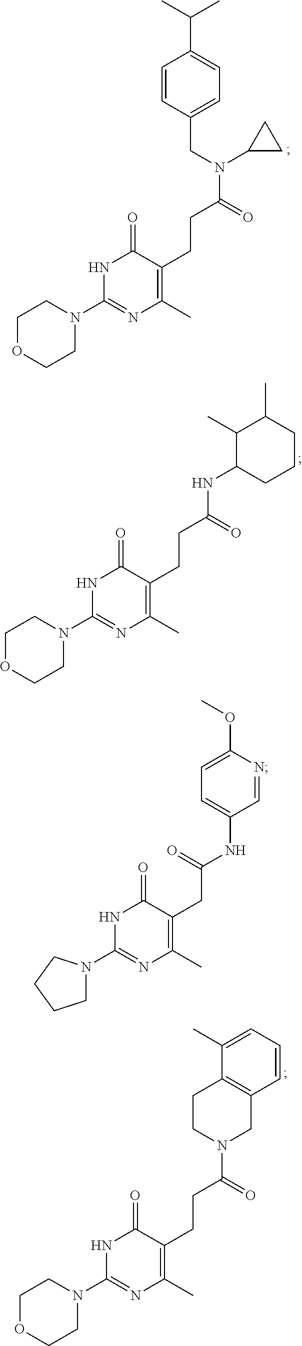 [SMILES: Cc1nc(N2CCOCC2)[nH]c(=O)c1CCC(=O)N(Cc1ccc(C(C)C)cc1)C1CC1 ; Cc1nc(N2CCOCC2)[nH]c(=O)c1CCC(=O)NC1CCCC(C)C1C ; COc1ccc(NC(=O)Cc2c(C)nc(N3CCCC3)[nH]c2=O)cn1 ; Cc1cccc2c1CCN(C(=O)CCc1c(C)nc(N3CCOCC3)[nH]c1=O)C2]

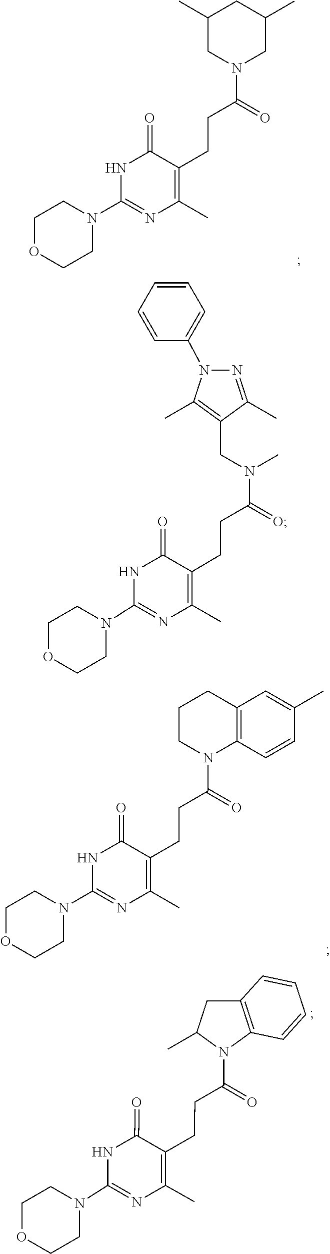 [SMILES: Cc1nc(N2CCOCC2)[nH]c(=O)c1CCC(=O)N1CC(C)CC(C)C1 ; Cc1nn(-c2ccccc2)c(C)c1CN(C)C(=O)CCc1c(C)nc(N2CCOCC2)[nH]c1=O ; Cc1ccc2c(c1)CCCN2C(=O)CCc1c(C)nc(N2CCOCC2)[nH]c1=O ; Cc1nc(N2CCOCC2)[nH]c(=O)c1CCC(=O)N1c2ccccc2CC1C]

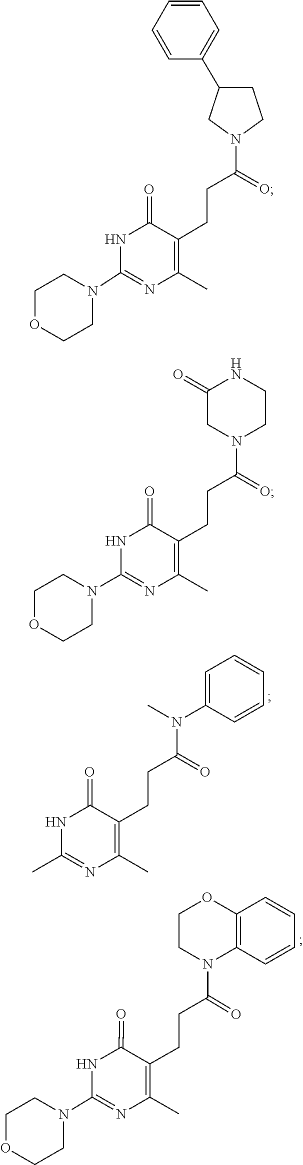 [SMILES: Cc1nc(N2CCOCC2)[nH]c(=O)c1CCC(=O)N1CCC(c2ccccc2)C1 ; Cc1nc(N2CCOCC2)[nH]c(=O)c1CCC(=O)N1CCNC(=O)C1 ; Cc1nc(C)c(CCC(=O)N(C)c2ccccc2)c(=O)[nH]1 ; Cc1nc(N2CCOCC2)[nH]c(=O)c1CCC(=O)N1CCOc2ccccc21]

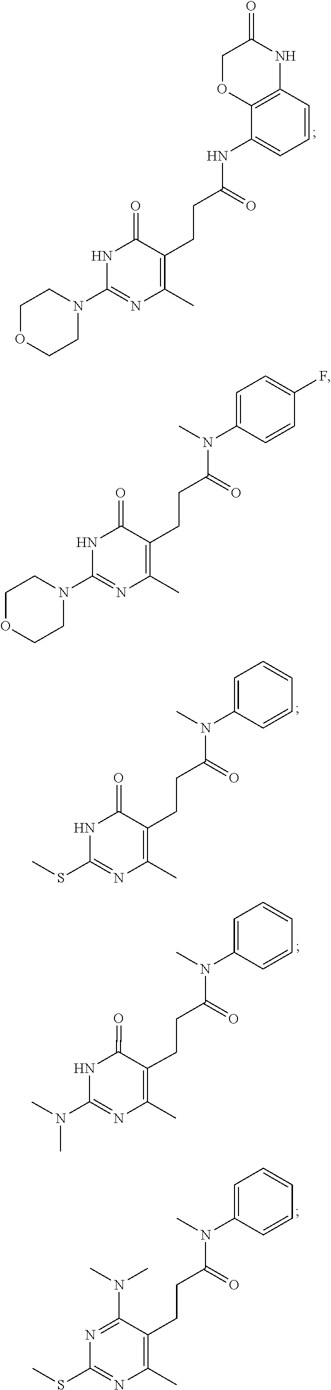 [SMILES: Cc1nc(N2CCOCC2)[nH]c(=O)c1CCC(=O)Nc1cccc2c1OCC(=O)N2 ; Cc1nc(N2CCOCC2)[nH]c(=O)c1CCC(=O)N(C)c1ccc(F)cc1 ; CSc1nc(C)c(CCC(=O)N(C)c2ccccc2)c(=O)[nH]1 ; Cc1nc(N(C)C)[nH]c(=O)c1CCC(=O)N(C)c1ccccc1 ; CSc1nc(C)c(CCC(=O)N(C)c2ccccc2)c(N(C)C)n1]

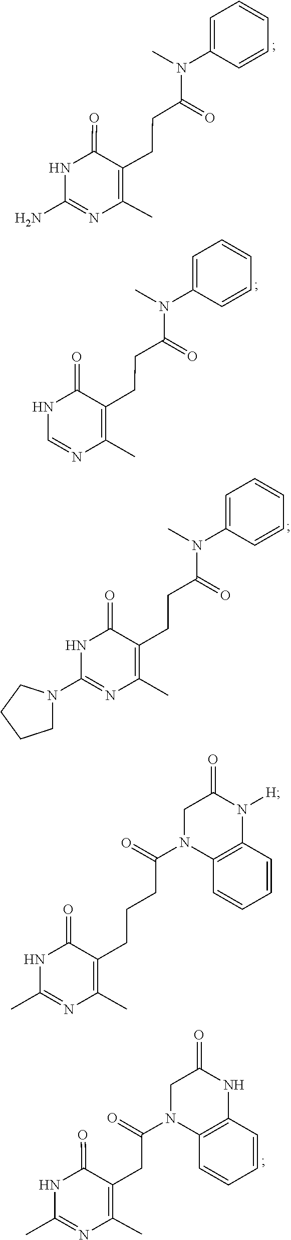

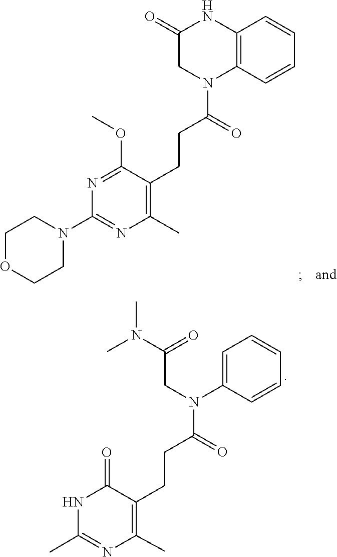 [SMILES: COc1nc(N2CCOCC2)nc(C)c1CCC(=O)N1CC(=O)Nc2ccccc21 ; Cc1nc(C)c(CCC(=O)N(CC(=O)N(C)C)c2ccccc2)c(=O)[nH]1]

15. A compound of formula (IV): 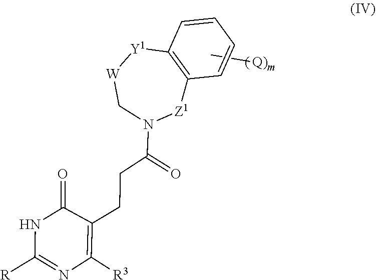
wherein:
R is selected from the group consisting of —H, C₁-C₃-alkyl, substituted C₁-C₃-alkyl, C₃-C₆-cycloalkyl, substituted C₃-C₆-cycloalkyl, heterocycyl, substituted heterocycyl, aryl, substituted aryl, —NHC(═O)R⁹, —N(R⁹)₂, —OR⁹, and —SR⁹;
R³ is selected from the group consisting of C₁-C₆-alkyl, C₃-C₆-cycloalkyl, and —CF₃;
m is 0, 1 or 2;
W is selected from the group consisting of —C(═O)— and —CH₂—;
each Q is independently selected from the group consisting of —F, —Cl, —CN, —CF₃ and C₁-C₃-alkyl;
Y¹ is selected from the group consisting of —NH—, —N(CH₃)—, —N(CH₂CH₃)— and —N(cyclopropyl)-;
each R⁹ is independently selected from the group consisting of —H, —CH₃, —CH₂CH₃ and cyclopropyl; and
Z¹ is absent or is —CH₂—;
or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof;
with the proviso that the compound is not:
4-[3-[1,6-dihydro-4-methyl-2-(4-morpholinyl)-6-oxo-5-pyrimidinyl]-1-oxopropyl]-3,4-dihydro-2(1H)-quinoxalinone, 6-methyl-2-(4-morpholinyl)-5-[3-oxo-3-(1,2,3,5-tetrahydro-1-methyl-4H-1,4-benzodiazepin-4-yl)propyl]-4(3H)-pyrimidinone, or 5-[3-(3,4-dihydro-4-methyl-1(2H)-quinoxalinyl)-3-oxopropyl]-6-methyl-2-(4-morpholinyl)-4(3H)-pyrimidinone.

16. The compound of claim 15, wherein Z¹ is absent.

17. The compound of claim 15, wherein m is 1.

18. The compound of claim 15, wherein m is 0.

19. The compound of any of claims 15 to 18, wherein Q is —F, —CF₃, or —CH₃.

20. The compound of any of claims 15 to 19, wherein W is —C(═O)—.

21. The compound of any of claims 15 to 20, wherein Y¹ is —NH—.

22. The compound of any of claims 15 to 20, wherein Y¹ is —NCH₃—.

23. The compound of any of claims 15 to 22, wherein R is selected from the group consisting of —H, C₁-C₃-alkyl, C₃-C₆-cycloalkyl, heterocyclyl, aryl, —NHC(═O)R⁹, —N(R⁹)₂, —OR⁹, and —SR⁹.

24. The compound of any of claims 15 to 23, wherein R is phenyl.

25. The compound of any of claims 15 to 23, wherein R is a heterocyclyl.

26. The compound of claim 25, wherein R is azetidinyl, pyrrolidinyl or piperidinyl.

27. The compound of claim 25, wherein R is morpholinyl.

28. The compound of any of claims 15 to 23, wherein R is —N(CH₃)₂ or —N(CH₂CH₃)₂.

29. The compound of any of claims 15 to 23, wherein R is —OCH₃, —OCH₂CH₃, —SCH₃ or —SCH₂CH₃.

30. The compound of any of claims 15 to 23, wherein R is C₁-C₃-alkyl or C₃-C₆-cycloalkyl.

31. The compound of claim 30, wherein R is —CH₃.

32. The compound of claim 30, wherein R is —CH₂CH₃.

33. The compound of claim 30, wherein R is cyclopropyl.

34. The compound of any of claims 15 to 33, wherein R³ is —CF₃.

35. The compound of any of claims 15 to 33, wherein R³ is —CH₃.

36. The compound of claim 15, wherein the compound is selected from the group consisting of:

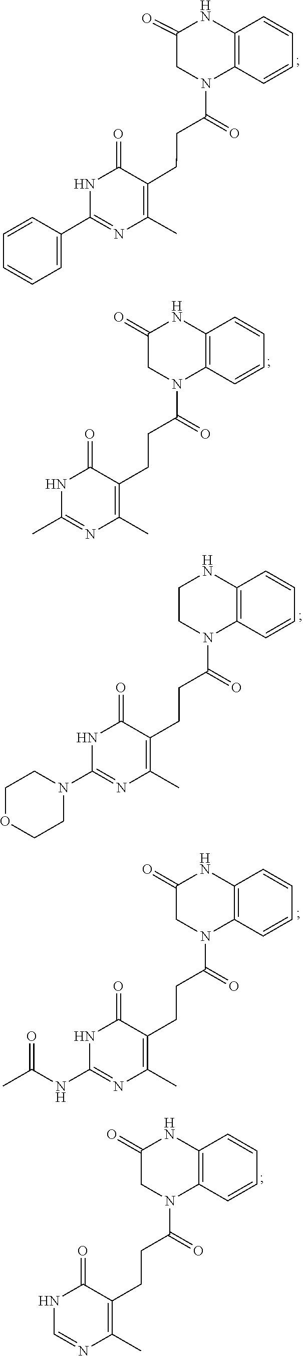 [SMILES: Cc1nc(-c2ccccc2)[nH]c(=O)c1CCC(=O)N1CC(=O)Nc2ccccc21 ; Cc1nc(C)c(CCC(=O)N2CC(=O)Nc3ccccc32)c(=O)[nH]1 ; Cc1nc(N2CCOCC2)[nH]c(=O)c1CCC(=O)N1CCNc2ccccc21 ; CC(=O)Nc1nc(C)c(CCC(=O)N2CC(=O)Nc3ccccc32)c(=O)[nH]1 ; Cc1nc[nH]c(=O)c1CCC(=O)N1CC(=O)Nc2ccccc21]

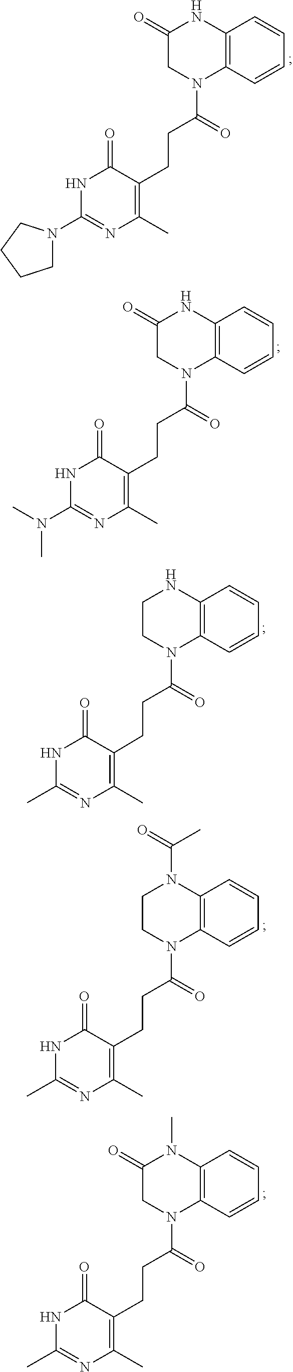

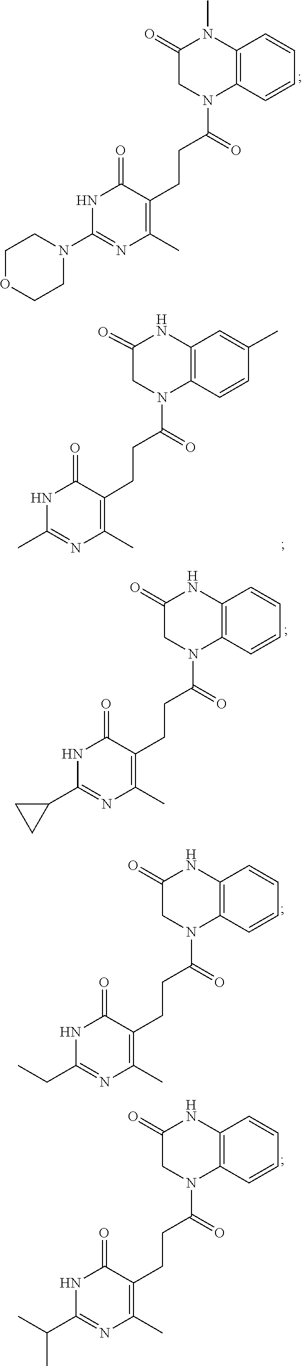

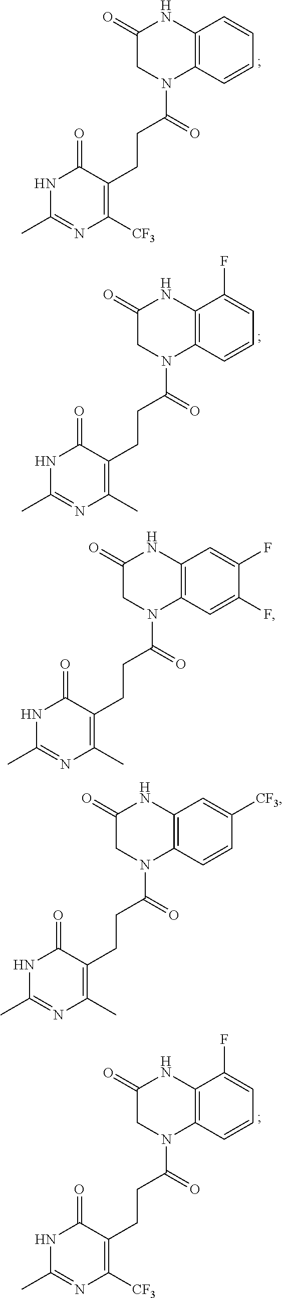

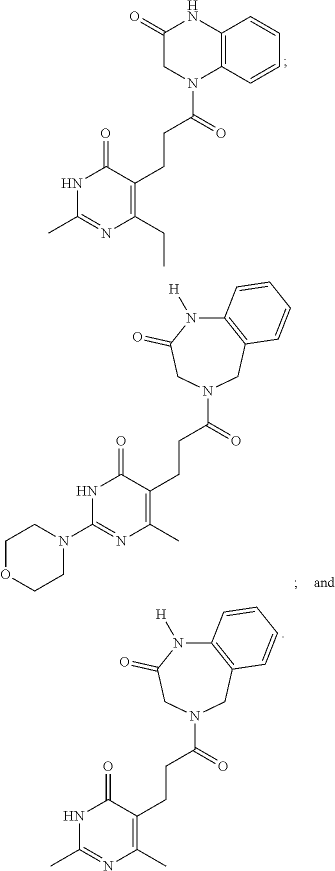

37. A method of inhibiting activity of an amylin receptor, the method comprising:
administering to a subject in need thereof, a therapeutically effective amount of a compound of formula (IV) of claim 15.

38. The method of claim 37, wherein the administering is effective for reducing cyclic AMP signal production in a cell.

39. The method of claim 37, wherein the amylin receptor is an AMY3 receptor.

40. The method of claim 37, wherein the administering is effective for producing a neuroprotective effect against amylin and/or amyloid-beta protein induced neurotoxicity.

41. The method of claim 37, wherein the administering is effective for treating a disease mediated through activity of the amylin receptor.

42. The method of claim 41, wherein the disease is Alzheimer's disease.
